(19) United States
(12) Patent Application Publication
(10) Pub. No.: US 2020/0281202 A1
(43) Pub. Date: Sep. 10, 2020

(54) NOVEL FUNGICIDAL HETEROCYCLIC COMPOUNDS

(71) Applicant: PI INDUSTRIES LTD., Udaipur Rajasthan (IN)

(72) Inventors: Gajanan Shanbhag, Karnataka (IN); Aditya Sharma, Rajasthan (IN); Yuvraj Navanath Kale, Maharashtra (IN); G. Renugadevi, Chennai (IN); Jagadish Pabba, Rajasthan (IN); Singaraboena Prabhakar, Warangal (IN); Mohan lal Mehta, Rajasthan (IN); Mohan Kumar S.P., Karnataka (IN); Yogesh Kashiram Belkar, Maharashtra (IN); Santosh Shridhar Autkar, Maharashtra (IN); Ruchi Garg, Uttar Pradesh (IN); Hagalavadi M. Venkatesha, Karnataka (IN); Alexander Guenther Maria Klausener, Pulheim (DE)

(73) Assignee: PI INDUSTRIES LTD., Udaipur Rajasthan (IN)

(21) Appl. No.: 16/644,978

(22) PCT Filed: Aug. 29, 2018
(86) PCT No.: PCT/IB2018/056581

§ 371 Date: Mar. 6, 2020

(30) Foreign Application Priority Data
Sep. 8, 2017 (IN) 201711031859

(51) Int. Cl. A01N 43/56; A01N 43/40; C07D 213/64; C07D 231/20

(52) U.S. Cl. CPC A01N 43/56; C07D 231/20; C07D 213/64; A01N 43/40

(57) ABSTRACT

The present invention relates to a compound selected from Formula I and a process for preparing the same,

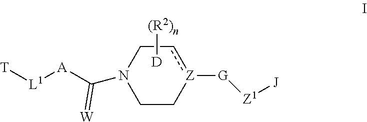

wherein, R², T, L¹, A, G, J, n, W, Z and Z¹ are each as defined in the description.

The invention also relates to the combination and composition comprising the compound of Formula I.

### FIELD OF THE INVENTION

The present invention relates to novel fungicidal heterocyclic compounds and it's salts, metal complexes, N-oxides, enantiomers, stereoisomers and polymorphs thereof; compositions and methods of use of the compounds for controlling or preventing phytopathogenic micro-organisms.

### BACKGROUND

The control of damages to crops caused by phytopathogenic micro-organisms is extremely important in achieving high crop efficiency. For instance, plant disease damage to ornamental, vegetable, field, cereal, and fruit crops can cause significant reduction in productivity and thereby result in increased costs to the consumer. Many products are commercially available to control such damages. The need continues for new compounds which are more effective, less costly, less toxic and environmentally safer and/or have different modes of action.

Certain oxazole-thiazole piperdine heterocyclic compounds having fungicidal properties have already been described in the literature, as for example, WO2008013622, WO2008013925, WO2009094407, WO2009094445, WO2010065579, WO2010123791, WO2011076699, WO2011085170, WO2012020060, WO2012025557, WO2012055837, WO2012082580, WO2012104273, WO2013037768, WO2013098229, WO2013127784, WO2013127808, WO2014075873, WO2014075874, WO2014118142, WO2014118143, WO2014154530, WO2014179144, WO2014206896, WO2015028457, WO2015144571, WO2016024350, WO2016024434, WO2017109855, WO2017109858, and WO2017138069.

The effectiveness of the oxazole-thiazole piperdine heterocyclic compounds described in the prior art is satisfactory, but leaves something to be desired in various cases. Therefore, it is always of high interest in agriculture to use novel pesticidal compounds in order to avoid and/or control the development of microorganisms such as fungal or bacterial pathogens or pests being resistant to known active ingredients. It is therefore of high interest to use novel compounds.

Surprisingly, it has been found now that the compounds and compositions thereof of the present invention have the potential of overcoming drawbacks and are suitable for crop protection against phytopathogenic micro-organisms causing plant diseases.

### SUMMARY OF THE INVENTION

The present invention relates to a compound selected from Formula I,

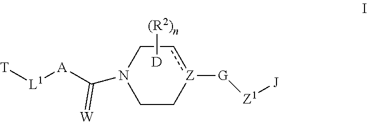

wherein, the substituents are as defined in the description.

The present invention will now be described in detail in the description.

### DETAILED DESCRIPTION OF THE INVENTION

### Definitions

The following definitions provided herein for the terminologies used in the present invention are for illustrative purpose only and in no manner limit the scope of the present invention.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

Compounds of the present invention may be present either in pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present invention. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The term “alkyl”, used either alone or in compound words such as “alkylthio” or “haloalkyl” or —N(alkyl) or alkylcarbonylalkyl or alkylsuphonylamino includes straight-chain or branched C₁ to C₂₄ alkyl, preferably C₁ to C₁₅ alkyl, more preferably C₁ to C₁₀ alkyl, most preferably C₁ to C₆ alkyl. Representative examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, carbonyl, carbonyloxy and the like, are at the end.

The term “alkenyl”, used either alone or in compound words includes straight-chain or branched C₂ to C₂₄ alkenes, preferably C₂ to C₁₅ alkenes, more preferably C₂ to C₁₀ alkenes, most preferably C₂ to C₆ alkenes. Representative examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, I-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, I-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

The term “alkynyl”, used either alone or in compound words includes straight-chain or branched C₂ to C₂₄ alkenes, preferably C₂ to C₁₅ alkynes, more preferably C₂ to C₁₀ alkynes, most preferably C₂ to C₆ alkynes. Representative examples of alkynes include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. “Alkynyl” can also include moieties comprised of multiple triple bonds such as 2,5-hexadiynyl.

The term “cycloalkyl” means alkyl closed to form a ring. Representative examples include but are not limited to cyclopropyl, cyclobutyl, cyclopentyl and cyclohexyl. This definition also applies to cycloalkyl as a part of a composite substituent, for example cycloalkylalkyl etc., unless specifically defined elsewhere.

The term “cycloalkenyl” means alkenyl closed to form a ring including monocyclic, partially unsaturated hydrocarbyl groups. Representative examples include but are not limited to cyclopentenyl and cyclohexenyl. This definition also applies to cycloalkenyl as a part of a composite substituent, for example cycloalkenylalkyl etc., unless specifically defined elsewhere.

The term “cycloalkynyl” means alkynyl closed to form a ring including monocyclic, partially unsaturated groups. This definition also applies to cycloalkynyl as a part of a composite substituent, for example cycloalkynylalkyl etc., unless specifically defined elsewhere.

The terms “cycloalkoxy”, “cycloalkenyloxy” and the like are defined analogously. Representative examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl” and “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The term “haloalkylthio” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Examples of“haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂.

The term “hydroxy” means —OH, the term “amino” means —NRR, wherein R can be H or any possible substituent such as alkyl. The term “carbonyl” means —C(O)—, the term “carbonyloxy” means —OC(O)—, the term “sulfinyl” means S(O), and the term “sulfonyl” means S(O)₂.

The term “alkoxy” used either alone or in compound words included C₁ to C₂₄ alkoxy, preferably C₁ to C₁₅ alkoxy, more preferably C₁ to C₁₀ alkoxy, most preferably C₁ to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

The term “alkoxyalkyl” means alkoxy substitution on alkyl. Examples of “alkoxyalkyl” include CH₃OCH₂; CH₃OCH₂CH₂; CH₃CH₂OCH₂; CH₃CH₂CH₂CH₂OCH₂ and CH₃CH₂OCH₂CH₂.

The term “alkoxyalkoxy” means alkoxy substitution on alkoxy.

The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

The terms “halocylcoalkyl”, “halocylcoalkenyl”, “alkylcycloalkyl”, “cycloalkylalkyl”, “cycloalkoxyalkyl”, “alkylsulfinylalkyl”, “alkylsulfonylalkyl”, “haloalkylcarbonyl”, “cycloalkylcarbonyl”, “haloalkoxylalkyl”, and the like, are defined analogously to the above examples.

The term “alkylthioalkyl” means alkylthio substitution on alkyl. Non-limiting examples of “alkylthioalkyl” include CH₂SCH₂; CH₂SCH₂CH₂; CH₃CH₂SCH₂; CH₃CH₂CH₂CH₂SCH₂; CH₃CH₂SCH₂CH₂ and the like or different isomers. The term “alkylthioalkoxy” denotes alkylthio substitution on alkoxy. The term “cycloalkylalkylamino” denotes cycloalkyl substitution on alkyl amino.

The terms “alkoxyalkoxyalkyl”, “alkylaminoalkyl”, “dialkylaminoalkyl”, “cycloalkylaminoalkyl”, “cycloalkylaminocarbonyl” and the like, are defined analogously to “alkylthioalkyl” or cycloalkylalkylamino.

The term “alkoxycarbonyl” is an alkoxy group bonded to a skeleton via a carbonyl group (—CO—). This definition also applies to alkoxycarbonyl as a part of a composite substituent, for example cycloalkylalkoxycarbonyl and the like, unless specifically defined elsewhere.

The term “alkoxycarbonylalkylamino” means alkoxy carbonyl substitution on alkyl amino. The term “alkylcarbonylalkylamino” means alkyl carbonyl substitution on alkyl amino. The terms alkylthioalkoxycarbonyl, cycloalkylalkylaminoalkyl and the like are defined analogously.

The term “alkylsulfinyl” means alkyl substitution on sulfinyl group. Non-limiting examples of “alkylsulfinyl” include methylsulphinyl; ethylsulphinyl; propylsulphinyl; 1-methylethylsulphinyl; butylsulphinyl; 1-methylpropylsulphinyl; 2-methylpropylsulphinyl; 1,1-dimethylethylsulphinyl; pentylsulphinyl; 1-methylbutylsulphinyl; 2-methylbutylsulphinyl; 3-methylbutylsulphinyl; 2,2-dimethylpropylsulphinyl; 1-ethylpropylsulphinyl; hexylsulphinyl; 1,1-dimethylpropylsulphinyl; 1,2-dimethylpropylsulphinyl; 1-methylpentylsulphinyl; 2-methylpentylsulphinyl; 3-methylpentylsulphinyl; 4-methylpentylsulphinyl; 1,1-dimethylbutylsulphinyl; 1,2-dimethylbutylsulphinyl; 1,3-dimethylbutylsulphinyl; 2,2-dimethylbutylsulphinyl; 2,3-dimethylbutylsulphinyl; 3,3-dimethylbutylsulphinyl; 1-ethylbutylsulphinyl; 2-ethylbutylsulphinyl; 1,1,2-trimethylpropylsulphinyl; 1,2,2-trimethylpropylsulphinyl; 1-ethyl-1-methylpropylsulphinyl; 1-ethyl-2-methylpropylsulphinyl and the like or different isomers. The term “arylsulfinyl” includes Ar—S(O), wherein Ar can be any carbocyle or heterocylcle. This definition also applies to alkylsulfinyl as a part of a composite substituent, for example haloalkylsulfinyl etc., unless specifically defined elsewhere.

The term “alkyl sulfonyl” means alkyl substitution on sulfonyl group. Non-limiting examples of “alkylsulfonyl” include methylsulphonyl; ethylsulphonyl; propylsulphonyl; 1-methylethylsulphonyl; butylsulphonyl; 1-methylpropylsulphonyl; 2-methylpropylsulphonyl; 1,1-dimethylethylsulphonyl; pentylsulphonyl; 1-methylbutylsulphonyl; 2-methylbutylsulphonyl; 3-methylbutylsulphonyl; 2,2-dimethylpropylsulphonyl; 1-ethylpropylsulphonyl; hexylsulphonyl; 1,1-dimethylpropylsulphonyl; 1,2-dimethylpropylsulphonyl; 1-methylpentylsulphonyl; 2-methylpentylsulphonyl; 3-methylpentylsulphonyl; 4-methylpentylsulphonyl; 1,1-dimethylbutylsulphonyl; 1,2-dimethylbutylsulphonyl; 1,3-dimethylbutylsulphonyl; 2,2-dimethylbutylsulphonyl; 2,3-dimethylbutylsulphonyl; 3,3-dimethylbutylsulphonyl; 1-ethylbutylsulphonyl; 2-ethylbutylsulphonyl; 1,1,2-trimethylpropylsulphonyl; 1,2,2-trimethylpropylsulphonyl; 1-ethyl-1-methylpropylsulphonyl; 1-ethyl-2-methylpropylsulphonyl and the like or different isomers. The term “arylsulfonyl” includes Ar—S(═O)₂, wherein Ar can be any carbocyle or heterocylcle. This definition also applies to alkylsulfonyl as a part of a composite substituent, for example alkylsulfonylalkyl etc., unless defined elsewhere.

The terms “alkylamino”, “dialkylamino”, and the like, are defined analogously to the above examples.

The term “carbocycle or carbocyclic” includes “aromatic carbocyclic ring system” and “nonaromatic carbocylic ring system” or polycyclic or bicyclic (spiro, fused, bridged, nonfused) ring compounds in which ring may be aromatic or non-aromatic (where aromatic indicates that the Hueckel rule is satisfied and non-aromatic indicates that the Hueckel rule is not statisfied).

The term “hetero” in connection with rings refers to a ring in which at least one ring atom is not carbon and which can contain heteroatoms independently selected from the group comprising of nitrogen, oxygen, sulfur, etc. The term “hetero” in connection with atom refer to an atom independently selected from nitrogen, sulfur, oxygen, etc.

The term “heterocycle” or “heterocyclic” includes “aromatic heterocycle” or “heteroaryl ring system” and “nonaromatic heterocycle ring system” or polycyclic or bicyclic (spiro, fused, bridged, non-fused) ring compounds in which ring may be aromatic or non-aromatic, wherein the heterocycle ring contains at least one heteroatom selected from N, O, S(═O)₀₋₂, and or C ring member of the heterocycle may be replaced by C(═O), C(═S), C(═CR*R*) and C(═NR*), * indicates integers.

The term “non-aromatic heterocyle” includes fused or unfused three- to fifteen-membered, preferably three- to twelve-membered, saturated or fully or partially unsaturated heterocycle, monocyclic or polycyclic (spiro, fused, bridged, nonfused) heterocycle wherein heteroatom is selected from the group of oxygen, nitrogen and sulphur; and if the ring contains more than one oxygen atom, they are not directly adjacent; Non-limiting examples of non-aromatic heterocyle include oxetanyl, oxiranyl; aziridinyl; thiiranyl, azetidinyl, thiethanyl, dithiethanyl, diazetidinyl, 2-tetrahydrofuranyl; 3-tetrahydrofuranyl; 2-tetrahydrothienyl; 3-tetrahydrothienyl; 2-pyrrolidinyl; 3-pyrrolidinyl; 3-isoxazolidinyl; 4-isoxazolidinyl; 5-isoxazolidinyl; 3-isothiazolidinyl; 4-isothiazolidinyl; 5-isothiazolidinyl; 3-pyrazolidinyl; 4-pyrazolidinyl; 5-pyrazolidinyl; 2-oxazolidinyl; 4-oxazolidinyl; 5-oxazolidinyl; 2-thiazolidinyl; 4-thiazolidinyl; 5-thiazolidinyl; 2-imidazolidinyl; 4-imidazolidinyl; 1,2,4-oxadiazolidin-3-yl; 1,2,4-oxadiazolidin-5-yl; 1,2,4-thiadiazolidin-3-yl; 1,2,4-thiadiazolidin-5-yl; 1,2,4-triazolidin-3-yl; 1,3,4-oxadiazolidin-2-yl; 1,3,4-thiadiazolidin-2-yl; 1,3,4-triazolidin-2-yl; 2,3-dihydrofur-2-yl; 2,3-dihydrofur-3-yl; 2,4-dihydrofur-2-yl; 2,4-dihydrofur-3-yl; 2,3-dihydrothien-2-yl; 2,3-dihydrothien-3-yl; 2,4-dihydrothien-2-yl; 2,4-dihydrothien-3-yl; 2-pyrrolin-2-yl; 2-pyrrolin-3-yl; 3-pyrrolin-2-yl; 3-pyrrolin-3-yl; 2-isoxazolin-3-yl; 3-isoxazolin-3-yl; 4-isoxazolin-3-yl; 2-isoxazolin-4-yl; 3-isoxazolin-4-yl; 4-isoxazolin-4-yl; 2-isoxazolin-5-yl; 3-isoxazolin-5-yl; 4-isoxazolin-5-yl; 2-isothiazolin-3-yl; 3-isothiazolin-3-yl; 4-isothiazolin-3-yl; 2-isothiazolin-4-yl; 3-isothiazolin-4-yl; 4-isothiazolin-4-yl; 2-isothiazolin-5-yl; 3-isothiazolin-5-yl; 4-isothiazolin-5-yl; 2,3-dihydropyrazol-1-yl; 2,3-dihydropyrazol-2-yl; 2,3-dihydropyrazol-3-yl; 2,3-dihydropyrazol-4-yl; 2,3-dihydropyrazol-5-yl; 3,4-dihydropyrazol-1-yl; 3,4-dihydropyrazol-3-yl; 3,4-dihydropyrazol-4-yl; 3,4-dihydropyrazol-5-yl; 4,5-dihydropyrazol-1-yl; 4,5-dihydropyrazol-3-yl; 4,5-dihydropyrazol-4-yl; 4,5-dihydropyrazol-5-yl; 2,3-dihydrooxazol-2-yl; 2,3-dihydrooxazol-3-yl; 2,3-dihydrooxazol-4-yl; 2,3-dihydrooxazol-5-yl; 3,4-dihydrooxazol-2-yl; 3,4-dihydrooxazol-3-yl; 3,4-dihydrooxazol-4-yl; 3,4-dihydrooxazol-5-yl; 3,4-dihydrooxazol-2-yl; 3,4-dihydrooxazol-3-yl; 3,4-dihydrooxazol-4-yl; 2-piperidinyl; 3-piperidinyl; 4-piperidinyl; 1,3-dioxan-5-yl; 2-tetrahydropyranyl; 4-tetrahydropyranyl; 2-tetrahydrothienyl; 3-hexahydropyridazinyl; 4-hexahydropyridazinyl; 2-hexahydropyrimidinyl; 4-hexahydropyrimidinyl; 5-hexahydropyrimidinyl; 2-piperazinyl; 1,3,5-hexahydrotriazin-2-yl; 1,2,4-hexahydrotriazin-3-yl; 2,3,4,5-tetrahydro[1H]azepin-1- or -2- or -3- or -4- or -5- or -6- or -7-yl; 3,4,5,6-tetra-hydro[2H]azepin-2- or -3- or -4- or -5- or -6- or -7-yl; 2,3,4,7-tetrahydro[1H]azepin-1- or -2- or -3- or -4- or -5- or -6- or -7-yl; 2,3,6,7-tetrahydro[1H]azepin-1- or -2- or -3- or -4- or -5- or -6- or -7-yl; hexahydroazepin-1- or -2- or -3- or -4-yl, tetra- and hexahydrooxepinyl such as 2,3,4,5-tetrahydro[1H]oxepin-2- or -3- or -4- or -5- or -6- or -7-yl; 2,3,4,7-tetrahydro[1H]oxepin-2- or -3- or -4- or -5- or -6- or -7-yl; 2,3,6,7-tetrahydro[1H]oxepin-2- or -3- or -4- or -5- or -6- or -7-yl; hexahydroazepin-1- or -2- or -3- or -4-yl; tetra- and hexahydro-1,3-diazepinyl; tetra- and hexahydro-1,4-diazepinyl; tetra- and hexahydro-1,3-oxazepinyl; tetra- and hexahydro-1,4-oxazepinyl, tetra- and hexahydro-1,3-dioxepinyl, tetra- and hexahydro-1,4-dioxepinyl. This definition also applies to heterocyclyl as a part of a composite substituent, for example heterocyclylalkyl etc., unless specifically defined elsewhere.

The term “aromatic heterocycle or heteroaryl” includes fused or unfused three to fifteen membered, preferably three to twelve membered, more preferably 5 or 6 membered; monocyclic or polycyclic unsaturated ring system, containing heteroatoms selected from the group of oxygen, nitrogen, sulphur, etc.

Non-limiting examples of 5 membered heteroaryl groups include furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited to) 1-pyrrolyl, 1-pyrazolyl, 1,2,4-triazol-1-yl, 1-imidazolyl, 1,2,3-triazol-1-yl and 1,3,4-triazol-1-yl.

Non-limiting examples of 6 membered heteroaryl groups include 2-pyridinyl; 3-pyridinyl; 4-pyridinyl; 3-pyridazinyl; 4-pyridazinyl; 2-pyrimidinyl; 4-pyrimidinyl; 5-pyrimidinyl; 2-pyrazinyl; 1,3,5-triazin-2-yl; 1,2,4-triazin-3-yl; 1,2,4,5-tetrazin-3-yl and the like.

Non-limiting examples of benzofused 5-membered heteroaryl include indol-1-yl; indol-2-yl; indol-3-yl; indol-4-yl; indol-5-yl; indol-6-yl; indol-7-yl; benzimidazol-1-yl; benzimidazol-2-yl; benzimidazol-4-yl; benzimidazol-5-yl; indazol-1-yl; indazol-3-yl; indazol-4-yl; indazol-5-yl; indazol-6-yl; indazol-7-yl; indazol-2-yl; 1-benzofuran-2-yl; 1-benzofuran-3-yl; 1-benzofuran-4-yl; 1-benzofuran-5-yl; 1-benzofuran-6-yl; 1-benzofuran-7-yl; 1-benzothiophen-2-yl; 1-benzothiophen-3-yl; 1-benzothiophen-4-yl; 1-benzothiophen-5-yl; 1-benzothiophen-6-yl; 1-benzothiophen-7-yl; 1,3-benzothiazol-2-yl; 1,3-benzothiazol-4-yl; 1,3-benzothiazol-5-yl; 1,3-benzothiazol-6-yl; 1,3-benzothiazol-7-yl; 1,3-benzoxazol-2-yl; 1,3-benzoxazol-4-yl; 1,3-benzoxazol-5-yl; 1,3-benzoxazol-6-yl; 1,3-benzoxazol-7-yl and the like.

Non-limiting examples of benzofused 6-membered heteroaryl include quinolin-2-yl; quinolin-3-yl; quinolin-4-yl; quinolin-5-yl; quinolin-6-yl; quinolin-7-yl; quinolin-8-yl; isoquinolin-1-yl; isoquinolin-3-yl; isoquinolin-4-yl; isoquinolin-5-yl; isoquinolin-6-yl; isoquinolin-7-yl; isoquinolin-8-yl and the like.

This definition also applies to heteroaryl as a part of a composite substituent, for example heteroarylalkyl etc., unless specifically defined elsewhere.

The term “aromatic heterocycle/heteroaryl” indicates that the Huckel's rule is satisfied and the term “non-aromatic heterocycle” indicates that the Huckel's rule is not satisfied.

The term “Huckel's rule” has the same meaning as defined and elaborated in Organic Chemistry by Jonathan Clayden, Nick Geeves, Stuart Warren.

The term “alkylsilyl” means branched and/or straight-chain alkyl radicals attached to a silicon atom. Non-limiting examples of alkylsilyl include trimethylsilyl, triethylsilyl, t-butyl-dimethylsilyl and the like or different isomers.

The term “haloalkylsilyl” means at least one alkyl radicals of alkylsilyl is partially or fully substituted with halogen atoms which may be the same or different.

The term “alkoxyalkylsilyl” denotes at least one alkyl radical of alkylsilyl is substituted with one or more alkoxy radicals which may be the same or different. The term “alkylsilyloxy” denotes an alkylsilyl moiety attached through oxygen.

The term “alkylcarbonyl” means alkyl group substituted on the carbonyl group. Non-limiting examples of “alkylcarbonyl” include C(O)CH₃, C(O)C₂CH₂CH₃ and C(O)CH(CH₃)₂.

The term “alkoxycarbonyl” means alkoxy group substituted on the carbonyl group. Non-limiting examples of “alkoxycarbonyl” include CH₃OC(═O), CH₃CH₂OC(═O), CH₃CH₂CH₂OC(═O), (CH₃)₂CHOC(═O) and the different butoxy or pentoxycarbonyl isomers.

The term “alkylaminocarbonyl” means alkylamino substituted on the carbonyl group. Non-limiting examples of “alkylaminocarbonyl” include CH₃NHC(═O), CH₃CH₂NHC(═O), CH₃CH₂CH₂NHC(═O), (CH₃)₂CHNHC(═O) and the different butylamino or pentylaminocarbonyl isomers.

The term “dialkylaminocarbonyl” means dialkylamino substituted on the carbonyl group. Non-limiting examples of “dialkylaminocarbonyl” include (CH₃)₂NC(═O), (CH₃CH₂)₂NC(═O), CH₃CH₂(CH₃)NC(═O), CH₁CH₂CH₂(CH₃)NC(═O) and (CH₃)₂CHN(CH₃)C(═O); and the like or different isomers.

Non-limiting examples of “alkoxyalkylcarbonyl” include CH₃OCH₂C(═O), CH₃OCH₂CH₂C(═O), CH₃CH₂OCH₂C(═O), CH₃CH₂CH₂CH₂OCH₂C(═O) and CH₃CH₂OCH₂CH₂C(═O) and the like or different isomers. Examples of “alkylthioalkylcarbonyl” include CH₃SCH₂C(═O), CH₃SCH₂CH₂C(═O), CH₃CH₂SCH₂C(═O), CH₃CH₂CH₂CH₂SCH₂C(═O) and CH₃CH₂SCH₂CH₂C(═O) and the like or different isomers. The term “haloalkylsufonylaminocarbonyl”, “alkylsulfonylaminocarbonyl”, “alkylthioalkoxycarbonyl”, “alkoxycarbonylalkylamino” and the like are defined analogously

Non-limiting examples of “alkylaminoalkylcarbonyl” include CH₃NHCH₂C(═O), CH₃NHCH₂CH₂C(═O), CH₃CH₂NHCH₂C(═O), CH₃CH₂CH₂CH₂NHCH₂C(═O) and CH₃CH₂NHCH₂CH₂C(═O) and the like or different isomers.

The term “amide” means A-R′C(═O)NR″—B, wherein R′ and R″ indicates substituents and A and B indicate any group.

The term “thioamide” means A-R′C(═S)NR″—B, wherein R′ and R″ indicates substituents and A and B indicate any group.

The total number of carbon atoms in a substituent group is indicated by the “Cᵢ to Cⱼ” prefix wherein i and j are numbers from 1 to 21. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of Formula I is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript m in (R)ₘ indicates an integer ranging from for example 0 to 4 then the number of substituents may be selected from the integers between 0 and 4 inclusive.

The groups defined herein above may further be substituted with any of the possible substitutent described herein above.

In any of the above recitations, when a compound of Formula I is comprised of one or more heterocyclic rings, the substituents may be attached to these rings through any available carbon or nitrogen by replacement of a hydrogen on said carbon or nitrogen.

In any of the above recitations, the substituents may be optionally further substituted.

When a compound is substituted with a substituent bearing a subscript that indicates the number of said substituents can exceed 1, said substituents (when they exceed 1) are independently selected from the group of defined substituents. Further, when the subscript “m” in (R)ₘ indicates an integer ranging from for example 0 to 4 then the number of substituents may be selected from the integers between 0 and 4 inclusive.

When a group contains a substituent which can be hydrogen, for example R₁ or R₂, then, when this substituent is taken as hydrogen, it is recognized that this is equivalent to said group being un-substituted.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the invention. It is not to be taken as an admission that any or all of these matters form a part of the prior art base or were common general knowledge in the field relevant to the invention as it existed anywhere before the priority date of this application.

The numerical values mentioned in the description and the claims though might form a critical part of the present invention, any deviation from such numerical values shall still fall within the scope of the present invention if that deviation follows the same scientific principle as that of the present invention.

The term “pest” for the purpose of the present invention includes but is not limited to fungi, stramenopiles (oomycetes), bacteria, nematodes, mites, ticks, insects and rodents.

The term “plant” is understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights.

For the purpose of the present invention the term “plant” includes a living organism of the kind exemplified by trees, shrubs, herbs, grasses, ferns, and mosses, typically growing in a site, absorbing water and required substances through its roots, and synthesizing nutrients in its leaves by photosynthesis.

Examples of “plant” for the purpose of the present invention include but are not limited to agricultural crops such as wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants. Preferably, the plant for the purpose of the present invention include but is not limited to cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and vegetables, ornamentals, any floricultural plants and other plants for use of human and animals.

The term “plant parts” is understood to mean all parts and organs of plants above and below the ground. For the purpose of the present invention the term plant parts includes but is not limited to cuttings, leaves, twigs, tubers, flowers, seeds, branches, roots including taproots, lateral roots, root hairs, root apex, root cap, rhizomes, slips, shoots, fruits, fruit bodies, bark, stem, buds, auxiliary buds, meristems, nodes and internodes.

The term “locus thereof” includes soil, surroundings of plant or plant parts and equipment or tools used before, during or after sowing/planting a plant or a plant part.

Application of a compound or compounds of the present invention or the compound of the present invention in a composition optionally comprising at-least one other active compatible compound to a plant or a plant material or locus thereof include application by a technique known to a person skilled in the art which include but is not limited to spraying, coating, dipping, fumigating, impregnating, injecting and dusting.

The term “applied” means adhered to a plant or plant part either physically or chemically.

The invention disclosed in the present invention shall now be elaborated with the help of non-limiting schemes and examples.

The present invention relates to a compound selected from Formula I,

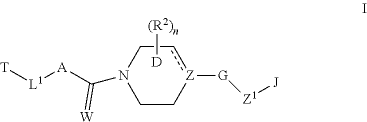

The present invention is inclusive of salts, metal complexes, N-oxides, isomers, and polymorphs of compound of Formula I.

T is selected from 5- or 6-membered aryl ring or 5- or 6-membered saturated or partially saturated cyclic ring or 5- or 6-membered heteroaryl ring or 5- or 6-membered saturated or partially saturated heterocyclic ring, wherein each ring member of heteroaryl ring is selected from C, N, O and S, and wherein each ring member of heterocyclic ring is selected from C, N, O, S(O)ₐ, C═O, C═S, S═NR⁶ and S(O)═NR⁶, and T is optionally substituted by one or more R^{1a} on carbon ring members and one or more R^{1b} on heteroatom ring members.

Non-limiting representative examples of T are depicted herein below.

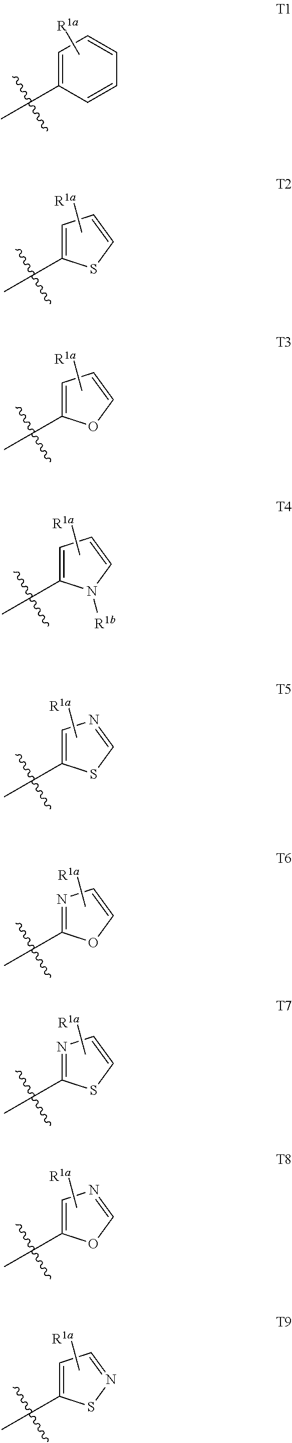

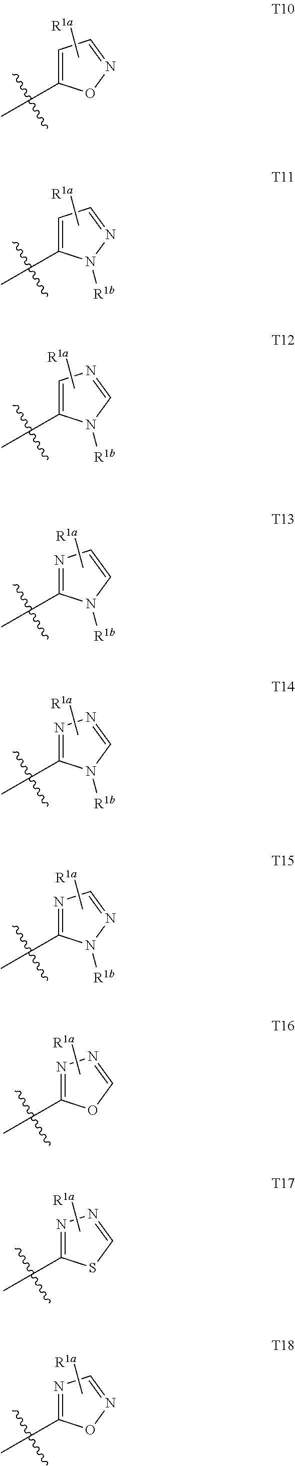

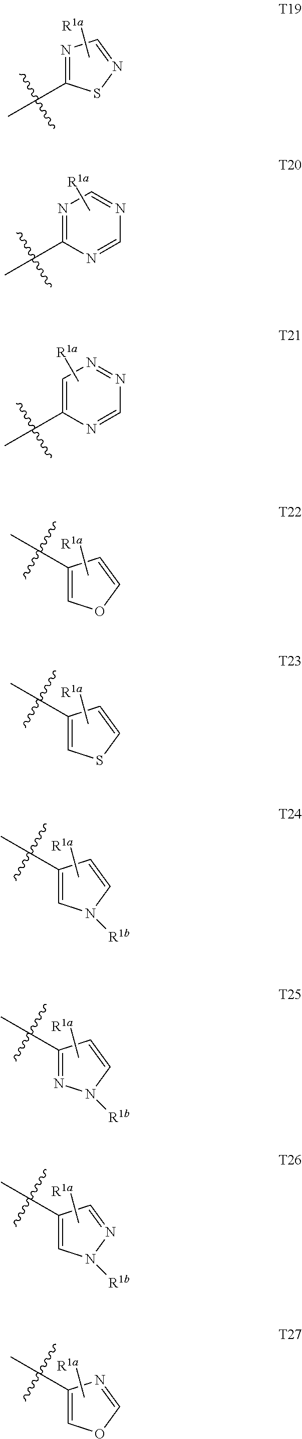

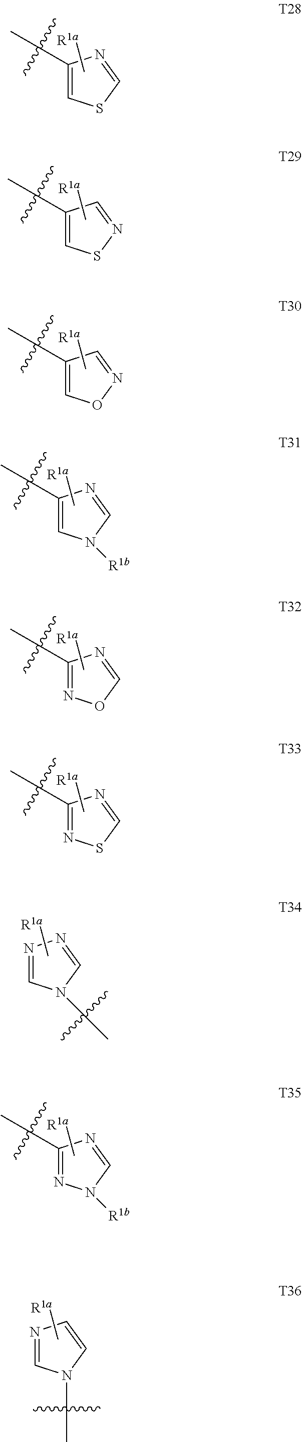

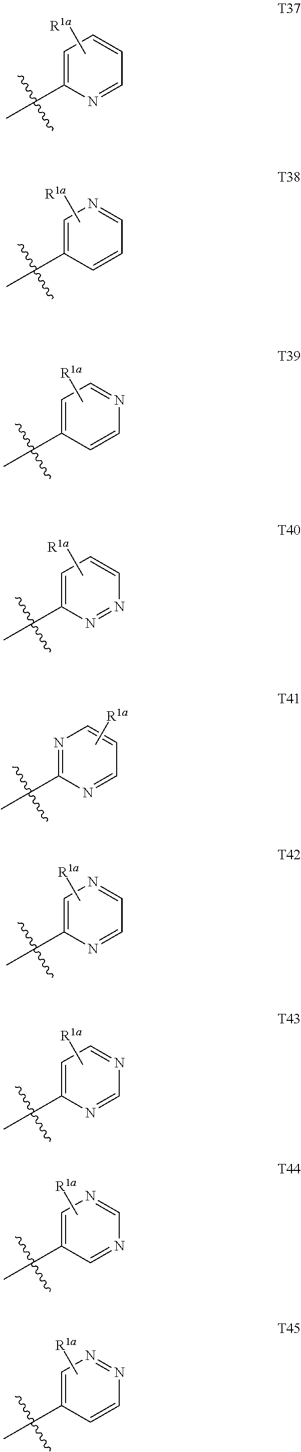

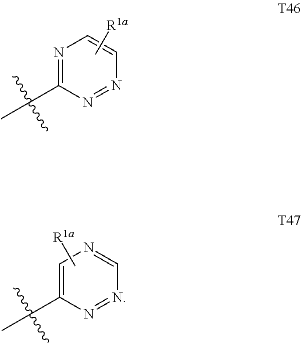

In one embodiment A is C(R¹⁵)₂ or C(R¹⁵)₂—C(R¹⁵)₂.

In another embodiment A is C(R¹⁵)₂.

In one embodiment the substituent R″ is independently selected from hydrogen, halogen, cyano, hydroxy, aldehyde, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylcarbonyl, C₁-C₆ haloalkylcarbonyl, C₁-C₆ alkoxycarbonyl, C₁-C₆ alkoxycarbonyl C₁-C₆ alkyl, C₁-C₆ alkylaminocarbonyl, C₁-C₆ dialkylaminocarbonyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆alkylsulfonyl and C₁-C₆ haloalkylsulfonyl.

In one embodiment Z is C or N. In one of the preferred embodiments Z is C.

In one embodiment the substitutent R² and R⁶ are independently selected from hydrogen, halogen, cyano, hydroxy, aldehyde, carboxylic acid, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₁-C₆ alkyl C₃-C₆ cycloalkyl, C₃-C₆ cycloalkyl C₁-C₆ alkyl, C₃-C₆ halocycloalkyl C₁-C₆alkyl, C₃-C₆ cycloalkenyl, C₃-C₆ halocycloalkenyl, C₁-C₆ alkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylamino C₁-C₆ alkyl, C₁-C₆ dialkylamino C₁-C₆ alkyl, C₁-C₆ haloalkylamino C₁-C₆ alkyl, C₁-C₆ alkylcarbonyl, C₁-C₆ haloalkylcarbonyl, C₃-C₆ cycloalkylcarbonyl, C₁-C₆ alkoxycarbonyl, C₃-C₆ cycloalkoxycarbonyl, C₃-C₆ cycloalkyl C₁-C₆ alkoxycarbonyl, C₁-C₆ alkylaminocarbonyl, C₁-C₆ dialkylaminocarbonyl, C₁-C₆ alkoxy, C₁-C₆ haloalkoxy, C₃-C₆ cycloalkoxy, C₃-C₆ halocycloalkoxy, C₂-C₆ alkenyloxy, C₂-C₆ haloalkenyloxy, C₂-C₆ alkynyloxy, C₂-C₆ haloalkynyloxy, C₁-C₆ alkoxy C₁-C₆ alkoxy, C₁-C₆ alkylcarbonyloxy, C₁-C₆ haloalkylcarbonyloxy, C₁-C₆ alkylthio, C₁-C₆ haloalkylthio, C₃-C₆ cycloalkylthio, C₁-C₆ alkylamino, C₁-C₆ dialkylamino, C₁-C₆ haloalkylamino, C₁-C₆ halodialkylamino, C₃-C₆ cycloalkylamino, C₁-C₆ alkylcarbonylamino, C₁-C₆ haloalkylcarbonylamino, C₁-C₆ alkylsulfonylamino and C₁-C₆ haloalkylsulfonylamino.

In another embodiment, two R² are taken together as C₁-C₄ alkylene or C₂-C₄ alkenylene or —CH═CH—CH═CH— to form a bridged bicyclic or fused bicyclic ring system optionally substituted with a substituent selected from C₁-C₆ alkyl, C₁-C₆ haloalkyl, C₁-C₆ alkoxy, C₁-C₆ haloalkoxy, halogen, hydroxy, amino, cyano and nitro.

In one of the preferred embodiments R² is selected from hydrogen, C₁-C₄ alkyl, C₂-C₄ alkenyl, C₂-C₄ alkynyl, C₁-C₄ haloalkyl, C₁-C₄ alkoxy, halogen, cyano and hydroxy.

G is an optionally substituted 5- or 6-membered heteroaryl ring or 5- or 6-membered saturated or partially saturated heterocyclic ring, each ring member of the heteroaryl ring is selected from C, N, O and S; and each ring member of the heterocyclic ring is selected from C, N, O, S(O)ₐ, C(═O), C(═S), S(═NR⁶) and S(O)═NR⁶, wherein, carbon ring members are substituted with one or more R^{3a} and heteroatom ring members are substituted with one or more R^{11a}.

In one of the embodiments G is an optionally substituted 5-membered heteroaryl.

In one of the preferred embodiments G is selected from optionally substituted G1 to G63, each substituent selected from R^{3a} on carbon ring members and R^{11a} on nitrogen ring members.

G1 to G63 are as depicted herein below:

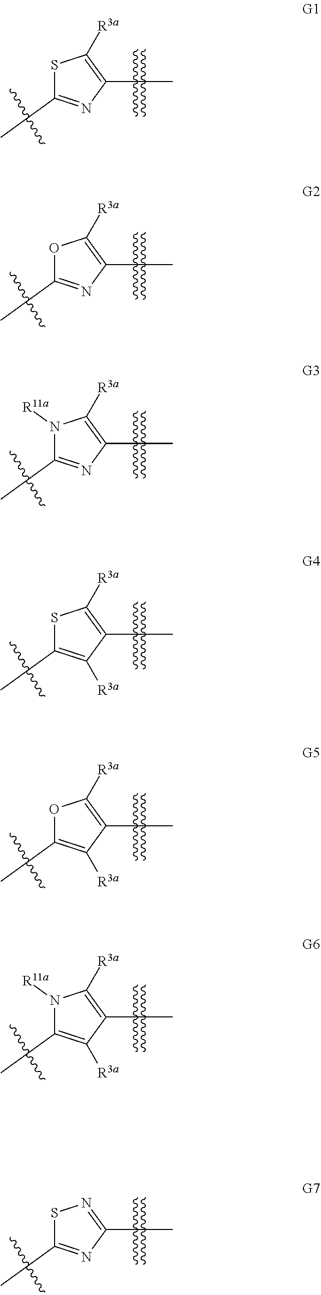

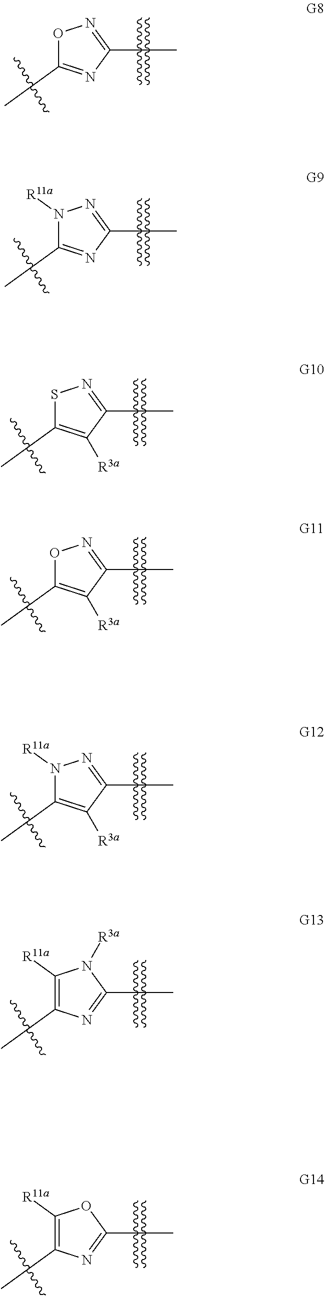

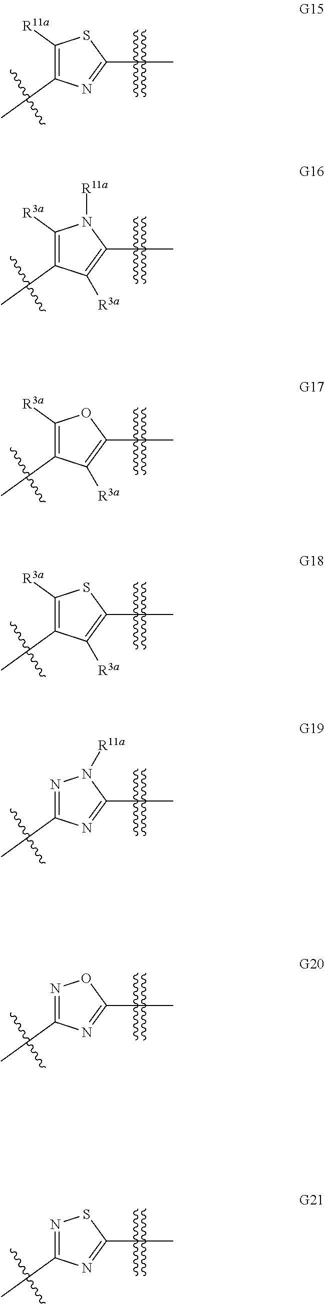

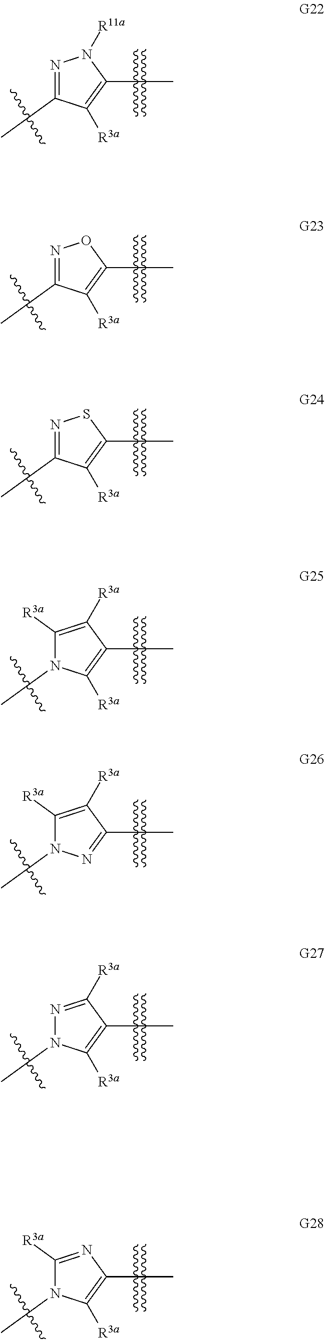

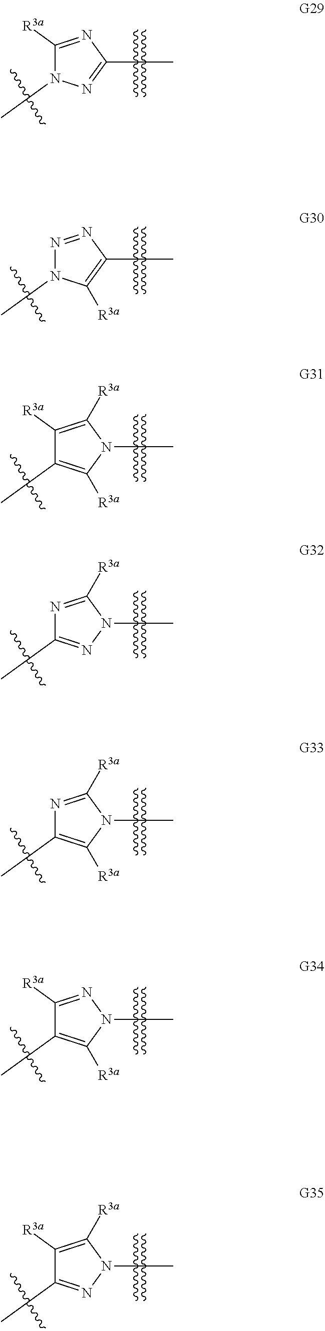

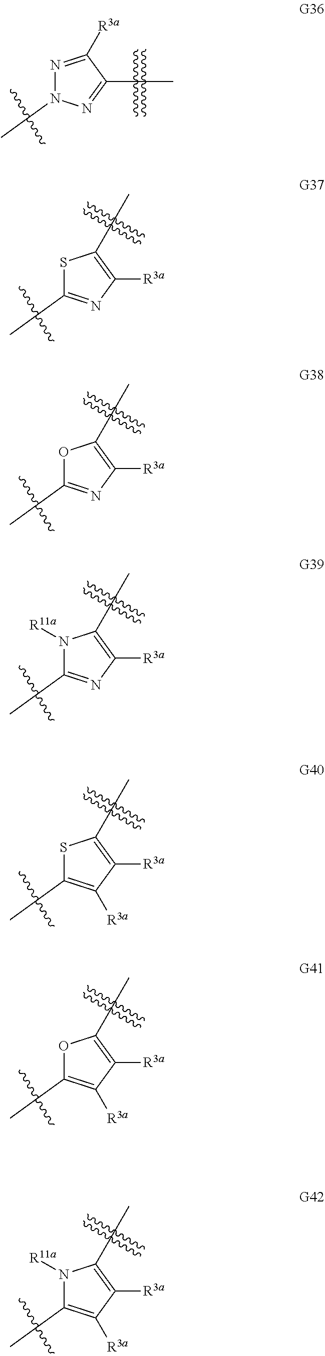

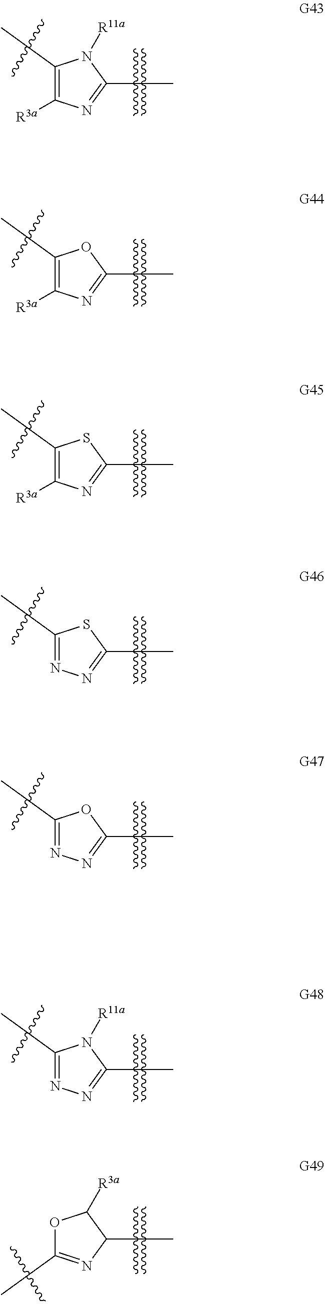

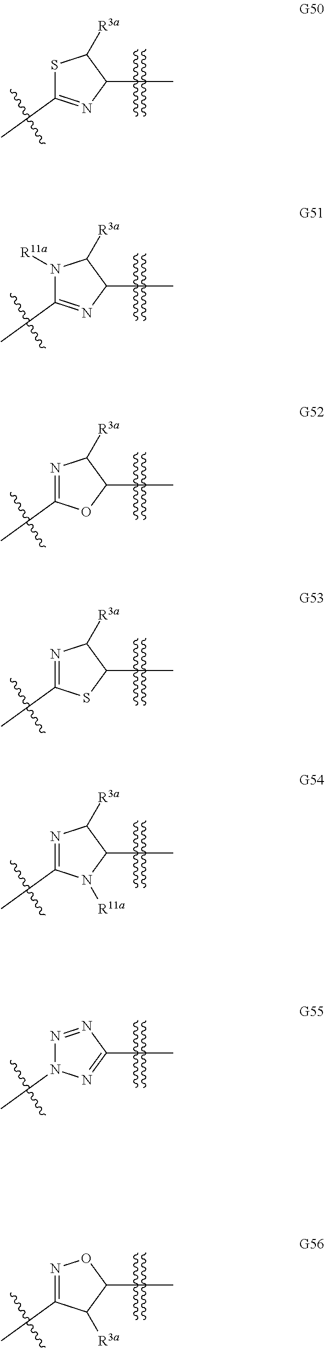

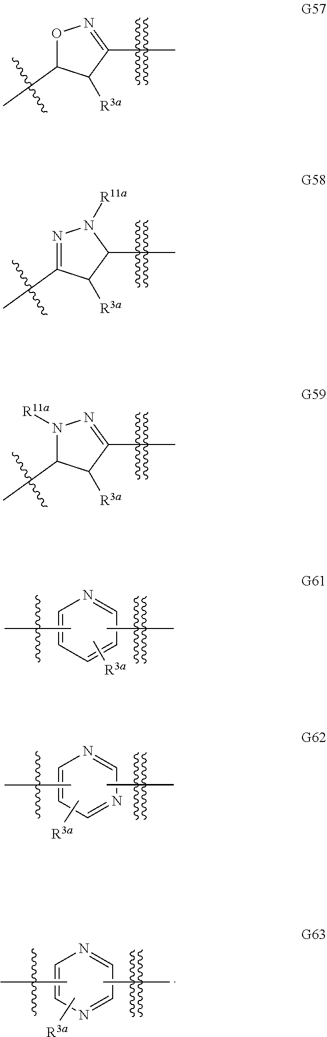

wherein the bond indicated by [symbol] is attached to ring D and the bond indicated by [symbol] is attached to J. R^{3a} and R^{11a} may be attached to one or more possible position/s.

The substituent R^{3a} is hydrogen or R^{3b}. The substituent R^{3b} is a phenyl or 5- or 6-membered heteroaromatic ring optionally substituted with one or more substituents independently selected from R^{4a} on carbon ring members and R^{4b} on nitrogen ring members. Alternatively, R^{3b} is independently C₁-C₃ alkyl, C₁-C₃ haloalkyl or halogen.

The substituent R^{4a} is independently selected from C₁-C₆ alkyl, C₂-C₆ alkenyl, C₂-C₆ alkynyl, C₃-C₆ cycloalkyl, C₃-C₆ cycloalkyl C₁-C₆ alkyl, C₁-C₆ alkyl C₃-C₆ cycloalkyl, C₁-C₆ haloalkyl, C₂-C₆ haloalkenyl, C₂-C₆ haloalkynyl, C₃-C₆ halocycloalkyl, halogen, hydroxy, amino, cyano, nitro, C₁-C₄ alkoxy, C₁-C₄ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₁-C₆ dialkylamino, C₃-C₆ cycloalkylamino, C₁-C₆ alkoxy C₁-C₆ alkyl, C₁-C₆ hydroxyalkyl, C₁-C₆ alkylcarbonyl, C₁-C₆ alkoxycarbonyl, C₁-C₆ alkylcarbonyloxy, C₁-C₆ alkylcarbonylthio, C₁-C₆ alkylaminocarbonyl, C₁-C₆ dialkylaminocarbonyl and C₁-C₆ trialkylsilyl.

The substituent R^{4b} is independently selected from C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl and C₁-C₆ alkoxy C₁-C₆ alkyl.

The substituent R^{11a} is hydrogen or R^{11b} and the substituent R^{11b} is independently selected from C₁-C₃ alkyl, C₃-C₆ cycloalkyl, C₁-C₆ haloalkyl, C₃-C₆ halocycloalkyl.

J is a 5-, 6- or 7-membered carbocylic or heterocyclic ring, a 8- to 11-membered carbocylic or heterocyclic bicyclic ring system or a 7- to 11-membered carbocylic or heterocyclic spirocyclic ring system, each ring member of the heteroyclir ring or ring system is selected from C, N, O, S(O)ₐ, C(═O), C(═S), and each ring or ring system is optionally substituted with one or more substituents independently selected from R⁵.

Particularly, J is a 5- or 6-membered heterocyclic ring, wherein heteroatom ring members are selected from N, O and S.

More particularly, J is a 5-membered heterocyclic ring, wherein heteroatom ring members are selected from N and O.

Alternatively, J is selected from

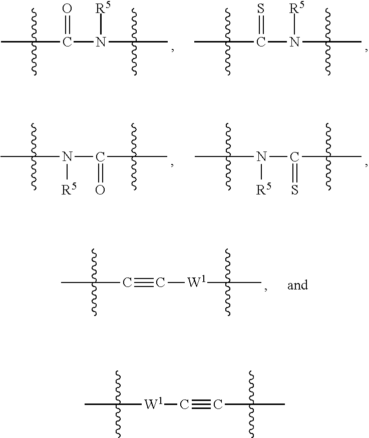

wherein W¹ is C(R⁵)₂ or CO or O or S or SO or SO₂ or NR⁶.

In one of the embodiments J is selected from J1 to J82 as depicted herein below:

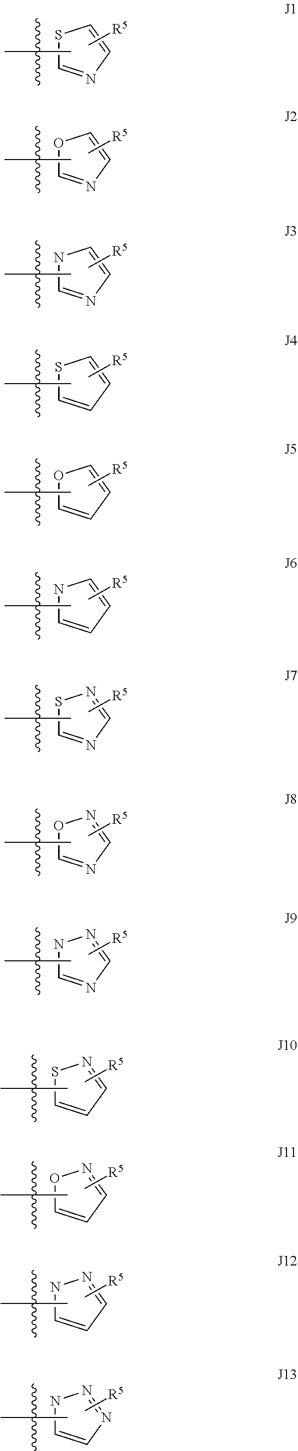

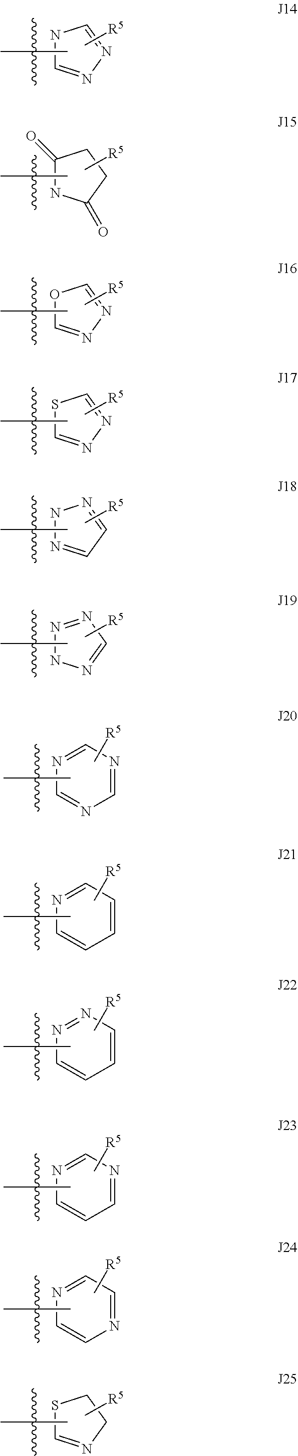

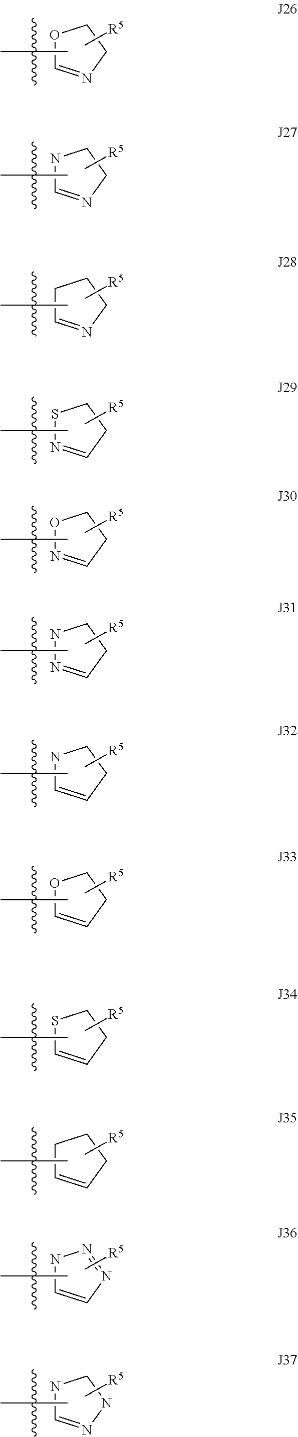

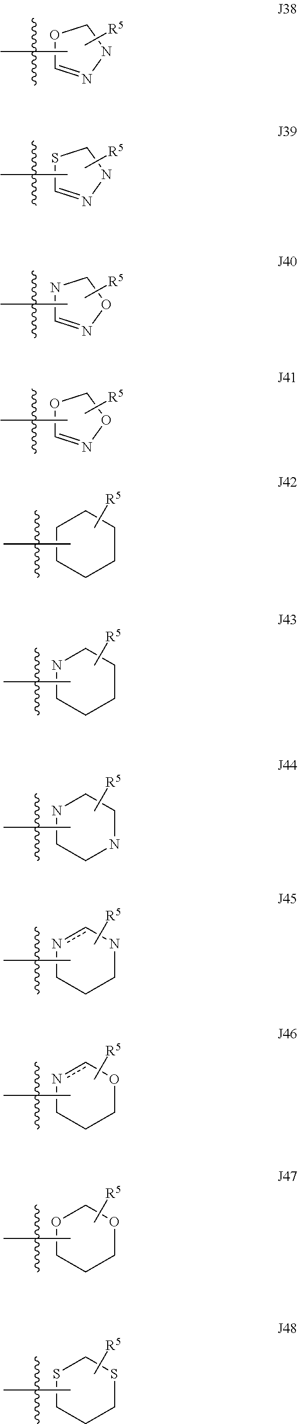

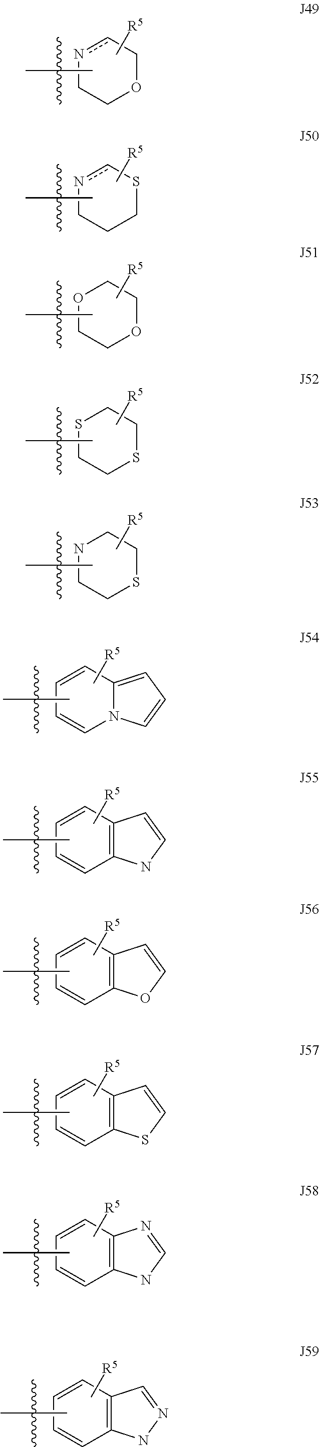

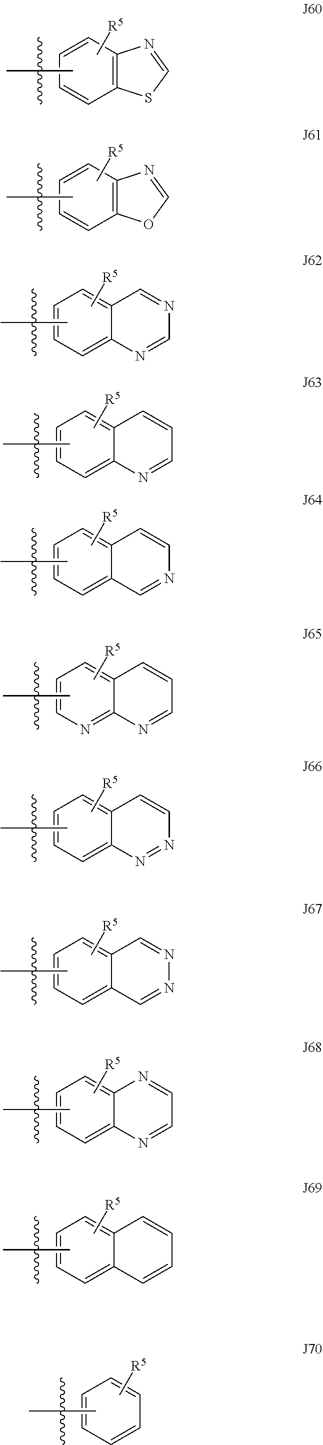

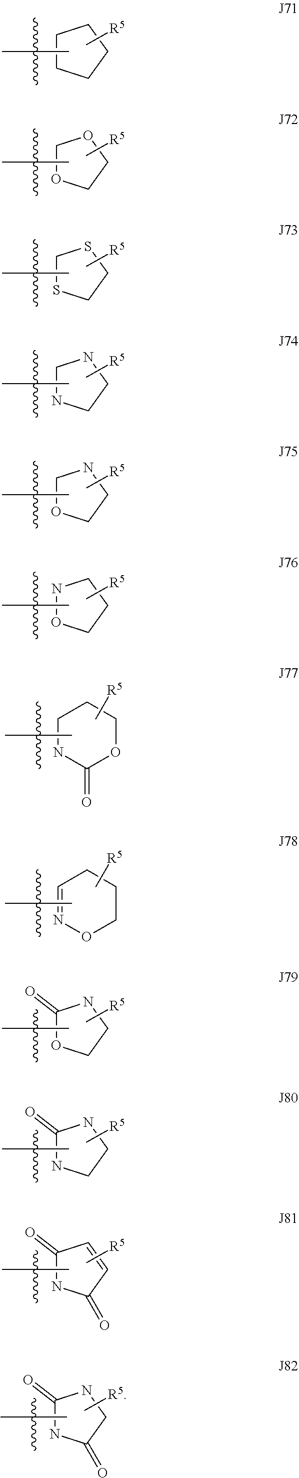

wherein the bond indicated by [symbol] is attached to Z¹; and R⁵ may be substituted at any of the possible position/s of J and the presentation “ [symbol] ” is a single or a double bond.

The subsituent R⁵ is independently selected from hydrogen, halogen, cyano, hydroxy, nitro, aldehyde, carboxylic acid, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₁-C₆ alkyl C₃-C₆ cycloalkyl, C₃-C₆ cycloalkyl C₁-C₆ alkyl, C₃-C₆ cycloalkyl C₃-C₆ cycloalkyl, C₃-C₆ halocyclo C₁-C₆ alkyl, C₃-C₆ cycloalkenyl, C₃-C₆ halocycloalkenyl, C₁-C₆ alkoxy C₁-C₆ alkyl, C₃-C₆ cycloalkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylamino C₁-C₆ alkyl, C₁-C₆ dialkylamino C₁-C₆ alkyl, C₁-C₆ haloalkylamino C₁-C₆ alkyl, C₁-C₆ cycloalkylamino C₁-C₆ alkyl, C₁-C₆ alkylcarbonyl, C₁-C₆ haloalkylcarbonyl, C₃-C₆ cycloalkylcarbonyl, C₁-C₆ alkoxycarbonyl, C₃-C₆ cycloalkoxycarbonyl, C₃-C₆ cycloalkyl C₁-C₆ alkoxycarbonyl, C₁-C₆ alkylaminocarbonyl, C₁-C₆ dialkylaminocarbonyl, C₃-C₆ cycloalkylaminocarbonyl, C₁-C₆ haloalkoxy C₁-C₆ alkyl, C₁-C₆ hydroxyalkyl, C₁-C₆ alkoxy, C₁-C₆ haloalkoxy, C₃-C₆ cycloalkoxy, C₃-C₆ halocycloalkoxy, C₃-C₆ cycloalkyl C₁-C₆ alkoxy, C₂-C₆ alkenyloxy, C₂-C₆ haloalkenyloxy, C₂-C₆ alkynyloxy, C₂-C₆ haloalkynyloxy, C₁-C₆ alkoxy C₁-C₆ alkoxy, C₁-C₆ alkylcarbonyloxy, C₁-C₆ haloalkylcarbonyloxy, C₃-C₆ cycloalkylcarbonyloxy, C₁-C₆ alkylcarbonyl C₁-C₆ alkoxy, C₁-C₆ alkylthio, C₁-C₆ haloalkylthio, C₃-C₆ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₆ cycloalkylsulfonyl, C₁-C₆ trialkylsilyl, C₁-C₆ alkylsulfonylamino, C₁-C₆ haloalkylsulfonylamino or —Z²Q.

Q is independently selected from phenyl, benzyl, naphthyl, a 5- or 6-membered aryl ring, an 8- to 11-membered aryl multi-cyclic ring system, an 8- to 11-membered aryl fused ring system, a 5- or 6-membered heteroaryl ring, an 8- to 11-membered heteroaryl multi-cyclic ring system or an 8- to 11-membered heteroaryl fused ring system, each ring member of the ring or the ring system is selected from C, N, O and S, and each ring or ring system is optionally substituted with one or more substituents independently selected from R⁷ on carbon atom ring members and R¹² on hetero atom ring members.

Alternatively, Q is independently selected from a 3- to 7-membered nonaromatic carbocyclic ring, a 5-, 6- or 7-membered nonaromatic heterocyclic ring, an 8- to 15-membered nonaromatic multi-cyclic ring system or an 8- to 15-membered nonaromatic fused ring system, each ring member of the ring or the ring system is selected from C, N, O, S(O)ₐ, C(═O), C(═S), S(═NR⁶) and S(═O)═NR⁶ & SiR¹⁶R^{11a}, and each ring or ring system is optionally substituted with one or more substituents independently selected from R⁷ on carbon atom ring members and R¹² on hetero atom ring members. The carbon to which Q is attached may be chiral or non-chiral carbon.

In one of the preferred embodiments, Q is selected from Q1 to Q99 and the presentation “ [symbol] ” is a single or a double bond. The substituent R¹⁴ may be attached to one or more position/s.

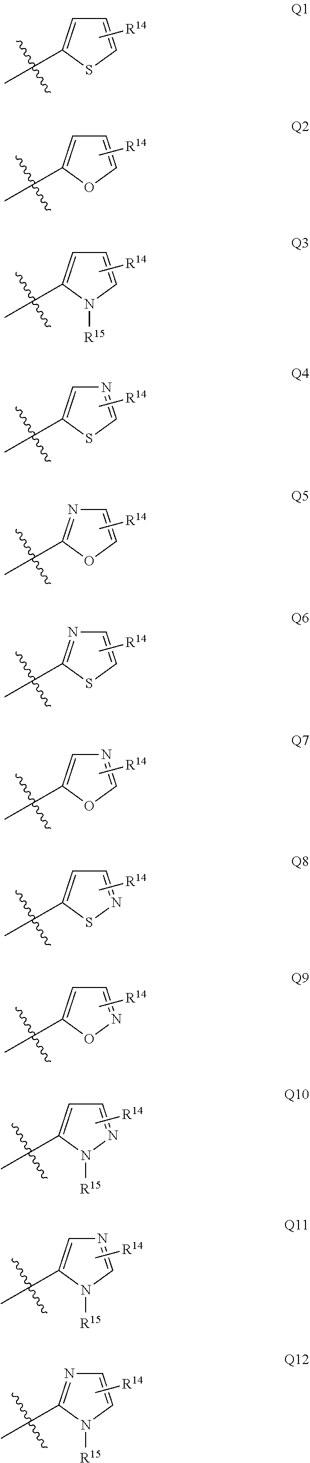

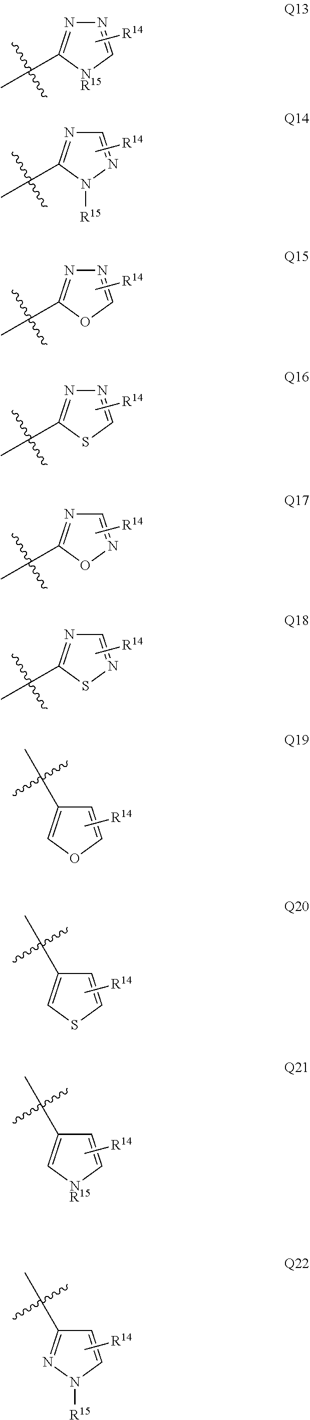

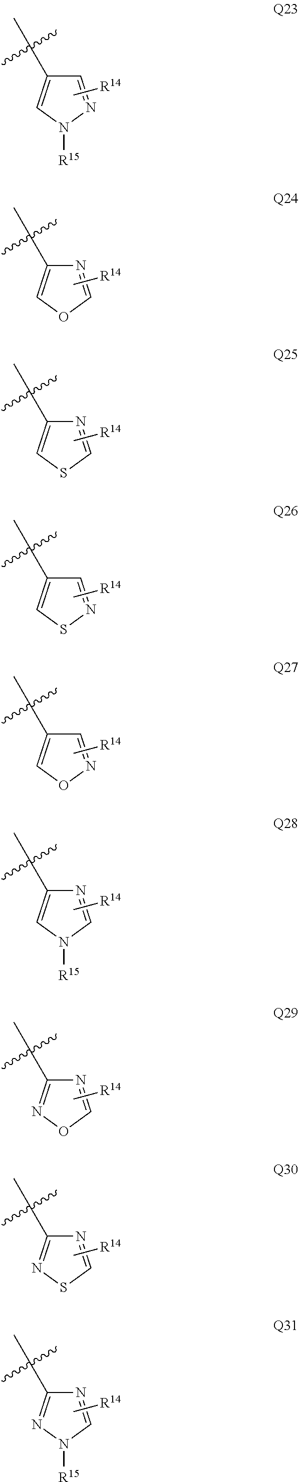

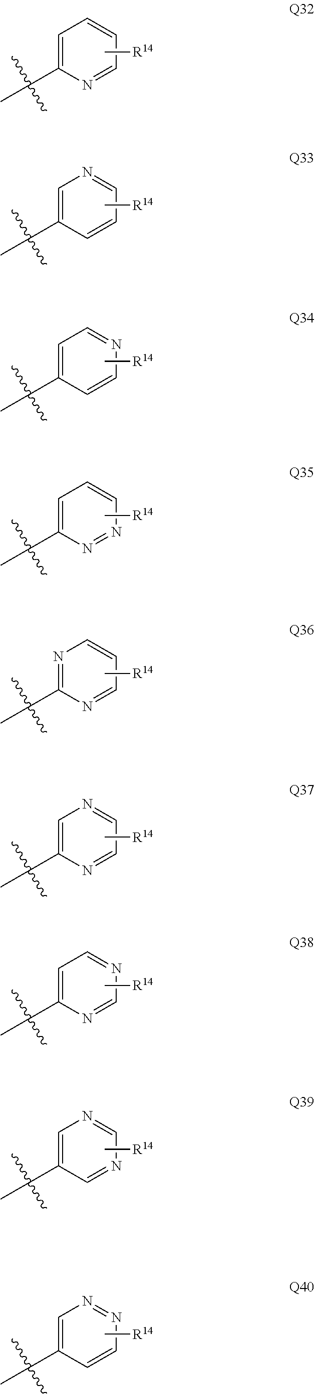

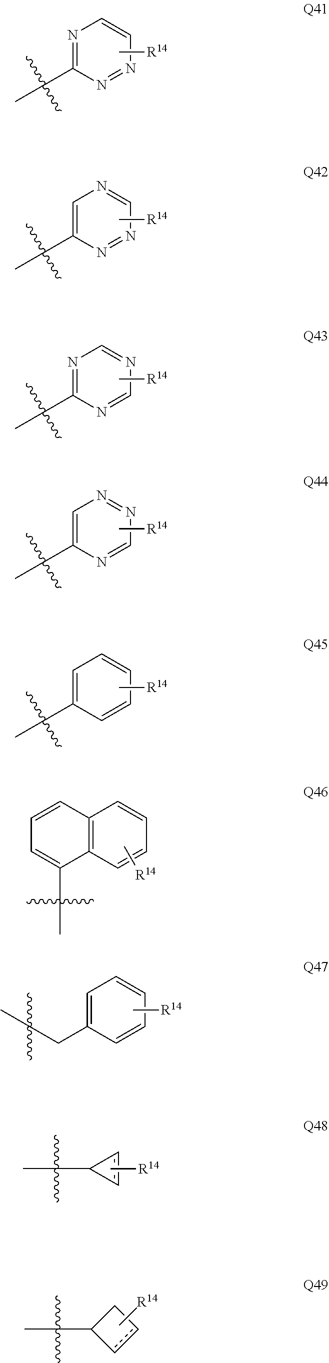

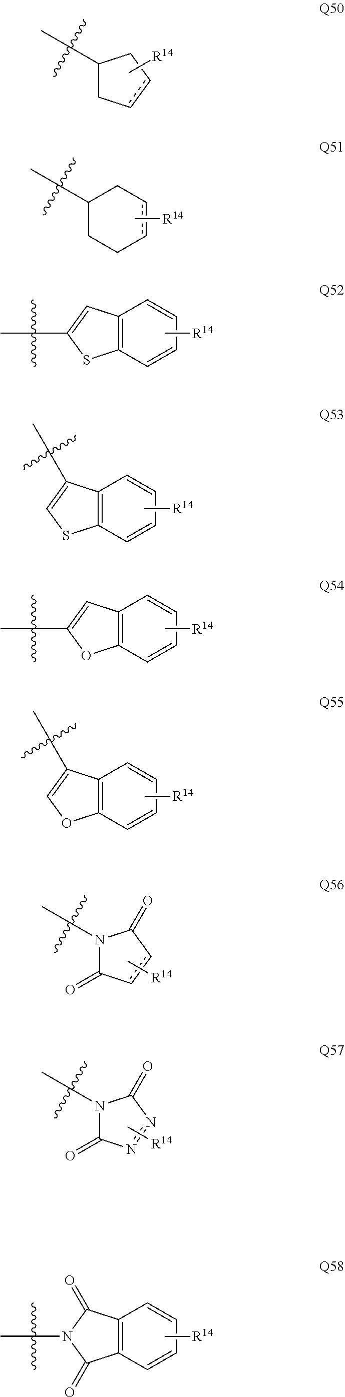

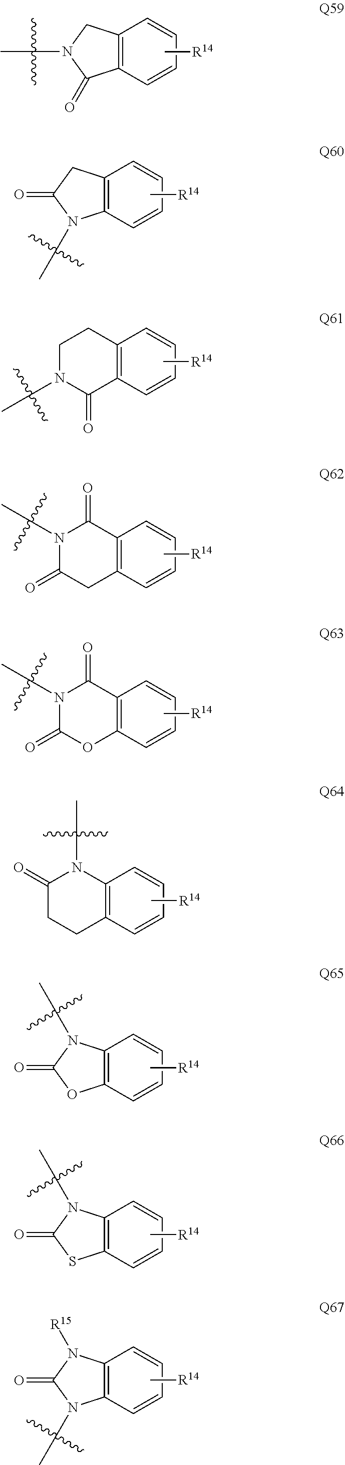

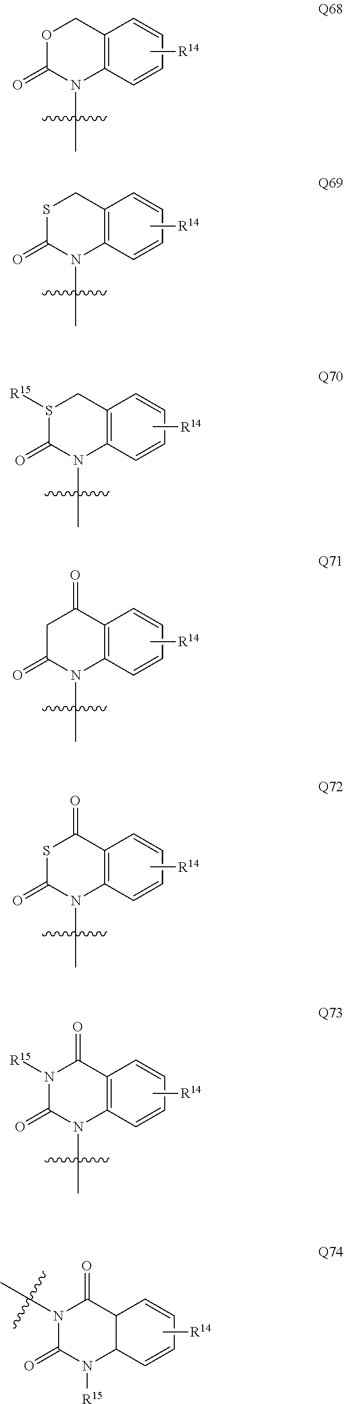

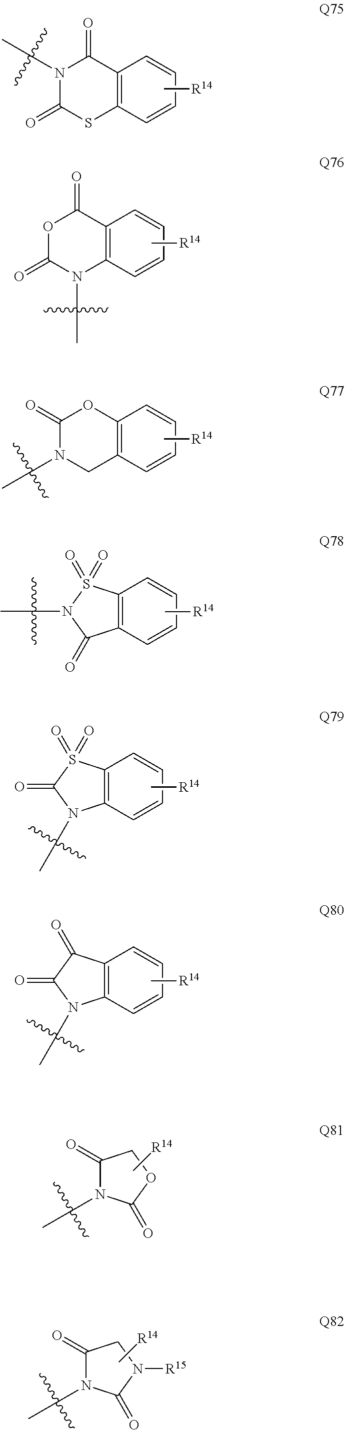

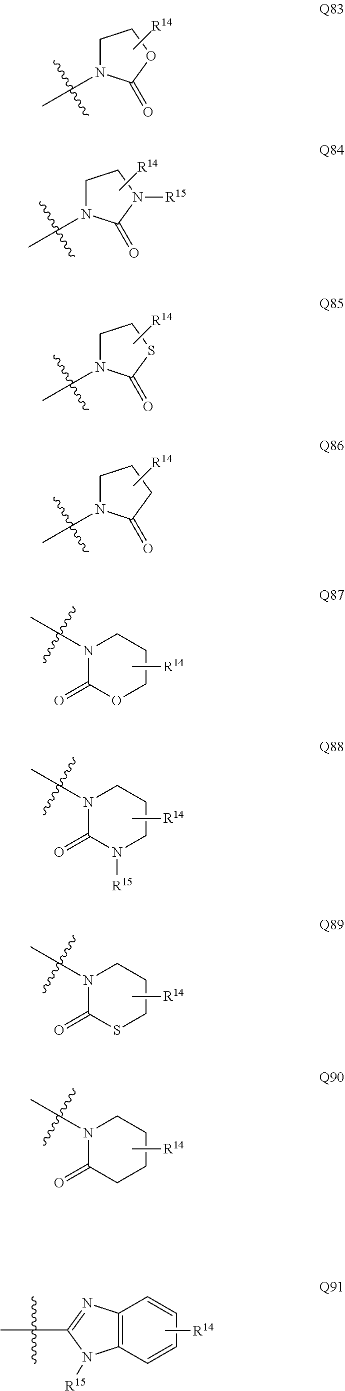

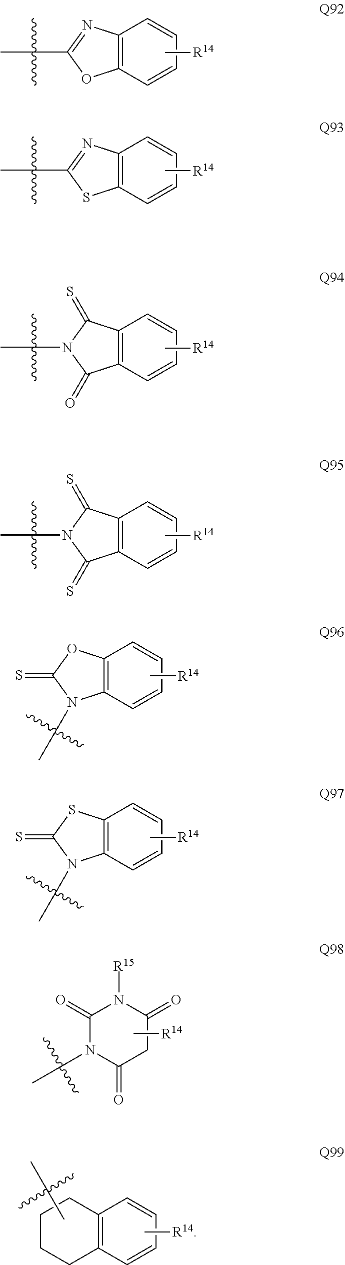

wherein the bond indicated by [symbol] is attached to J or Z².

Alternatively, J & Q together forms carbocyclic or heterocyclic dioxepine ring system.

In one of the preferred embodiments J & Q together form a fragment selected from M1 and M2:

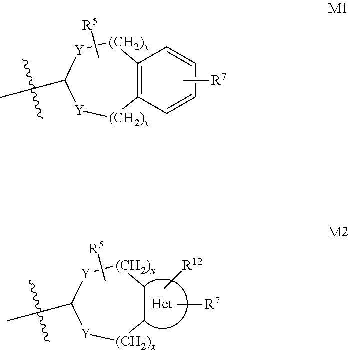

wherein, the substituents R¹, R⁷ and R¹² may be attached at one or more possible position/s, x in the fragments M1 and M2 is an integer ranging from 0 to 2 and Y is selected from N, O and S.

Particularly, J and Q together form a fragment selected from M1′ or M2′:

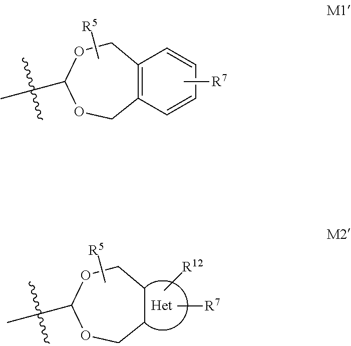

wherein, R⁵ and R⁷ each has the same meaning as defined.

The substituents R^{1a}, R^{1b}, R⁷ and R¹² are independently selected from hydrogen, halogen, hydroxy, cyano, nitro, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₁-C₆alkyl C₃-C₈cycloalkyl, C₃-C₈cycloalkyl C₁-C₆ alkyl, C₃₋₈ cycloalkyl C₃-C₈ cycloalkyl, C₃-C₈ halocycloalkyl C₁-C₆ alkyl, C₁-C₆ alkoxy C₁-C₆ alkyl, C₃-C₈ cycloalkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylamino, C₁-C₆ dialkylamino, C₁-C₆ alkylamino C₁-C₆ alkyl, C₁-C₆ dialkylamino C₁-C₆ alkyl, C₁-C₆ haloalkylamino C₁-C₆ alkyl, C₃-C₈ cycloalkylamino, C₃-C₈ cycloalkylamino C₁-C₆ alkyl, C₁-C₆ alkylcarbonyl, C₁-C₆ haloalkylcarbonyl, C₃-C₈ cycloalkylcarbonyl, C₁-C₆ alkoxycarbonyl, C₃-C₈ cycloalkoxycarbonyl, C₁-C₆ alkylaminocarbonyl, C₁-C₆ dialkylaminocarbonyl, C₃-C₈ cycloalkylaminocarbonyl, C₁-C₆ haloalkoxy C₁-C₆ alkyl, C₁-C₆ hydroxyalkyl, C₁-C₆ hydroxyalkenyl, C₁-C₆ hydroxyalkynyl, C₁-C₆ alkoxy, C₁-C₆ haloalkoxy, C₁-C₆ cycloalkoxy, C₃-C₈ halocycloalkoxy, C₃-C₈ cycloalkyl C₁-C₆ alkoxy, C₂-C₆ alkenyloxy, C₂-C₆ haloalkenyloxy, C₂-C₆ alkynyloxy, C₂-C₆ haloalkynyloxy, C₁-C₆ alkoxy C₁-C₆ alkoxy, C₁-C₆ alkylcarbonyloxy, C₁-C₆ haloalkylcarbonyloxy, C₃-C₆ cycloalkylcarbonyloxy, C₁-C₆ alkylcarbonyl C₁-C₆ alkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ cycloalkylsulfinyl, C₁-C₆ trialkylsilyl, C₁-C₆ alkylsulfonylamino, C₁-C₆ haloalkylsulfonylamino, C₁-C₆ alkylcarbonylthio, C₁-C₆ alkylsulfonyloxy, C₁-C₆ alkylsulfinyloxy, arylsulfonyloxy, arylsulfinyloxy, arylsulfonyl, arylsulfinyl, C₁-C₆ cyanoalkyl, C₂-C₆ alkenylcarbonyloxy, C₁-C₆ alkoxy C₁-C₆ alkylthio, C₁-C₆ alkylthio C₁-C₆ alkoxy, C₂-C₆ haloalkenylcarbonyloxy, C₁-C₆ alkoxy C₂-C₆ alkynyl, C₂-C₆ alkynylthio, C₃-C₈ halocycloalkylcarbonyloxy, C₂-C₆ alkenylamino, C₂-C₆ alkynylamino, C₁-C₆ haloalkylamino, C₃-C₈ cycloalkyl C₁-C₆ alkylamino, C₁-C₆ alkoxyamino, C₁-C₆ haloalkoxyamino, C₁-C₆ alkylcarbonylamino, C₁-C₆ haloalkylcarbonylamino, C₁-C₆ alkoxycarbonylamino, C₂-C₆ alkenylthio, C₁-C₆ haloalkoxycarbonyl, C₁-C₆ alkoxy C₁-C₆ alkylcarbonyl, C₁-C₆ haloalkoxycarbonylamino, C₁-C₆ alkoxy C₁-C₆ alkylaminocarbonyl, C₁-C₆ alkylthiocarbonyl, C₃-C₈ cycloalkenyloxy C₁-C₆ alkyl, C₁-C₆ alkoxy C₁-C₆ alkoxycarbonyl, C₁-C₆ haloalkoxy C₁-C₆ haloalkoxy, C₁-C₆ alkoxy C₁-C₆ haloalkoxy, C₃-C₈ halocycloalkoxy C₁-C₆ alkyl, C₁-C₆ dialkylaminocarbonylamino, C₁-C₆ alkoxy C₂-C₆ alkenyl, C₁-C₆ alkylthiocarbonyloxy, C₁-C₆ haloalkoxy C₁-C₆ alkoxy, C₁-C₆ haloalkylsulfonyloxy, C₁-C₆ alkoxy C₁-C₆ haloalkyl, C₁-C₆ dihaloalkylamino, C₁-C₆ dialkoxy C₁-C₆ alkyl, C₁-C₆ alkylaminocarbonylamino, C₁-C₆ haloalkoxy C₁-C₆ haloalkyl, C₁-C₆ alkylaminocarbonyl C₁-C₆ alkylamino, C₁-C₆ trialkylsilyl C₂-C₆ alkynyloxy, C₁-C₆ trialkylsilyloxy, C₁-C₆ trialkylsilyl C₂-C₆ alkynyl, C₁-C₆ cyanoalkoxy C₁-C₆ alkyl, C₁-C₆ dialkylthio C₁-C₆ alkyl, C₁-C₆ alkoxysulfonyl, C₃-C₈ halocycloalkoxycarbonyl, C₁-C₆ alkylcy C₃-C₈ cloalkylcarbonyl, C₃-C₈ halocyclo C₁-C₆ alkylcarbonyl, C₂-C₆ alkenyloxycarbonyl, C₂-C₆ alkynyloxycarbonyl, C₁-C₆ cyanoalkoxycarbonyl, C₁-C₆ alkylthio C₁-C₆ alkoxycarbonyl, C₂-C₆ alkynylcarbonyloxy, C₂-C₆ haloalkynylcarbonyloxy, cyanocarbonyloxy, C₁-C₆ cyanoalkylcarbonyloxy, C₃-C₈ cycloalkylsulphonyloxy, C₃-C₈ cycloalkyl C₁-C₆ alkylsulphonyloxy, C₃-C₈ halocycloalkylsulphonyloxy, C₂-C₆ alkenylsulphonyloxy, C₂-C₆ alkynylsulphonyloxy, C₁-C₆ cyanoalkylsulphonyloxy, C₂-C₆ haloalkenylsulphonyloxy, C₂-C₆ haloalkynylsulphonyloxy, C₂-C₆ alkynylcycloalkyloxy, C₂-C₆ cyanoalkenyloxy, C₂-C₆ cyanoalkynyloxy, C₁-C₆ alkoxycarbonyloxy, C₂-C₆ alkenyloxycarbonyloxy, C₂-C₆ alkynyloxycarbonyloxy, C₁-C₆ alkoxyalkylcarbonyloxy, sulfilimines, sulfoximines, SF₅ or Z²Q.

The substituents R¹⁶ and R¹⁷ are independently selected from C₁-C₆ alkyl, C₂-C₆ alkenyl, C₂-C₆ alkynyl, C₃-C₆ cycloalkyl, C₃-C₆ halocycloalkyl, C₁-C₆ cycloalkyl C₁-C₆ alkyl, C₁-C₆ alkyl C₃-C₆ cycloalkyl, C₁-C₆ haloalkyl, C₁-C₆alkoxy and C₁-C₆ haloalkoxy.

R⁵ and R⁷ or R⁵ and R¹² taken together with the atoms linking R⁵ and R⁷ or R¹² to form a saturated, unsaturated or partially unsaturated 4- to 7-membered ring, each ring members selected from C, N, O, S(O)ₐ, C═O, C═S, S═NR⁶ and S(O)═NR⁶, and said ring optionally substituted on ring members other than the atoms linking R⁵ and R⁷ or R¹² with R⁸.

R⁸ is selected from halogen, C₁-C₆ alkyl, C₁-C₆ haloalkyl, C₃-C₈ cycloalkyl, and C₃-C₈ cycloalkyl.

W is O or S. Preferably W is O.

The substituents Z¹ and Z² are independently a direct bond, O, C═O, C═S, S(O)ₐ, CHR²⁰ or NR²¹.

The substituent R²⁰ is independently hydrogen, C₁-C₄ alkyl or C₁-C₄ haloalkyl. The substituent R²¹ is independently hydrogen, C₁-C₈ alkyl, C₁-C₅ haloalkyl, C₃-C₈ cycloalkyl, C₁-C₆ alkylcarbonyl, C₁-C₈ haloalkylcarbonyl, C₁-C₈ alkoxycarbonyl or C₁-C₈ haloalkoxycarbonyl.

In one preferred embodiments, Z¹ and Z² are a direct bond or O or S or C═O.

The presentation “ [symbol] ” in ring D is a single bond when Z is N. Further, the presentation ‘ [symbol] ’ in ring D is a single or double bond when Z is C. In one of the preferred embodiment, the presentation “ [symbol] ” is a single bond.

“n” is an integer ranging from 0 to 9 with a provisos that when Z is N, “n” is an integer ranging from 0 to 8; and when the presentation “ [symbol] ” in ring D is a double bond then “n” is an integer ranging from 0 to 7.

L¹ is O, S, NR²³. In one of the preferred embodiments, L¹ is O.

The substituent R²³ is selected from hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylcarbonyl, C₁-C₆ haloalkylcarbonyl, C₁-C₆ alkoxycarbonyl, C₁-C₆ alkoxycarbonyl C₁-C₆ alkyl, C₁-C₆ alkylaminocarbonyl, C₁-C₆ dialkylaminocarbonyl, C₁-C₆ alkylsulfonyl and C₁-C₆ haloalkylsulfonyl.

a is independently 0, 1 or 2.

The following compounds are excluded from the definition of Formula I:
Ethanone, 1-[4-[4-(5-methyl-3-phenyl-4-isoxazolyl)-2-thiazolyl]-1-piperidinyl]-2-[[5-(trifluoromethyl)-2-pyridinyl]thio]-(CAS RN-1023141-80-1);
Benzamide, 2-[[2-[4-[4-[3-(3,4-dichlorophenyl)-5-isoxazolyl]-2-thiazolyl]-1-piperidinyl]-2-oxoethyl]thio]-4-ethoxy-(CAS RN-1177816-84-0);
Ethanone, 2-[(2-chloro-4-fluorophenyl)thio]-1-[4-[4-[3-(3,4-dichlorophenyl)-5-isoxazolyl]-2-thiazolyl]-1-piperidinyl]-(CAS RN-1177683-42-9);
Ethanone, 2-(cyclohexyloxy)-1-[4-[4-[5-(2,6-difluorophenyl)-4,5-dihydro-3-isoxazolyl]-2-thiazolyl]-1-piperidinyl]-(CAS RN-1173972-38-7);
1-Propanone, 2-(4-chlorophenoxy)-2-methyl-1-[4-[4-(5-methyl-3-phenyl-4-isoxazolyl)-2-thiazolyl]-1-piperidinyl]-(CAS RN-1136418-28-4);
Ethanone, 2-[(2-chloro-4-fluorophenyl)thio]-1-[4-[4-(5-methyl-3-phenyl-4-isoxazolyl)-2-thiazolyl]-1-piperidinyl]-(CAS RN-1023177-70-9);
Benzenesulfonamide, N-methyl-2-[[2-[4-[4-(5-methyl-3-phenyl-4-isoxazolyl)-2-thiazolyl]-1-piperidinyl]-2-oxoethyl]thio]-(CAS RN-1023156-55-9);
Benzenesulfonamide, 2-[[2-[4-[4-[3-(3,4-dichlorophenyl)-5-isoxazolyl]-2-thiazolyl]-1-piperidinyl]-2-oxoethyl]thio]-N-methyl-(CAS RN-1022602-51-2);
Ethanone, 1-[4-[4-(5-methyl-3-phenyl-4-isoxazolyl)-2-thiazolyl]-1-piperidinyl]-2-(2,3,4,5,6-pentafluorophenoxy)-(CAS RN-1022567-65-2);
Ethanone, 1-[4-[4-[3-(3,4-dichlorophenyl)-5-isoxazolyl]-2-thiazolyl]-1-piperidinyl]-2-[(4-methyl phenyl)sulfonyl]-(CAS RN-1022566-90-0);
Ethanone, 2-(2,4-dichlorophenoxy)-1-[4-[4-[3-(3,4-dichlorophenyl)-5-isoxazolyl]-2-thiazolyl]-1-piperidinyl]-(CAS RN-1022328-76-2);
Ethanone, 2-(2,4-dichlorophenoxy)-1-[4-[4-(5-methyl-3-phenyl-4-isoxazolyl)-2-thiazolyl]-1-piperidinyl]-(CAS RN-1022068-84-3);
Ethanone, 1-[4-[4-[3-(3,4-dichlorophenyl)-5-isoxazolyl]-2-thiazolyl]-1-piperidinyl]-2-(2,3,4,5,6-pentafluorophenoxy)-(CAS RN-1022028-25-6);
1-Propanone, 1-[4-[4-(5-methyl-3-phenyl-4-isoxazolyl)-2-thiazolyl]-1-piperidinyl]-3-[(2-methylphenyl)thio]-(CAS RN-1022326-33-5); and
1-Propanone, 1-[4-[4-[3-(3,4-dichlorophenyl)-5-isoxazolyl]-2-thiazolyl]-1-piperidinyl]-3-[(2-methylphenyl)thio]-(CAS RN 1024410-18-1).

The novel and inventive compounds of the present invention, the salts, isomers, metal complexes, N-oxides and polymorphs thereof are effective in preventing against and controlling phytopathogenic micro-organisms.

An anion part of the salt in case the compound of Formula I is cationic or capable of forming a cation can be inorganic or organic.

Alternatively, a cation part of the salt in case the compound of Formula I is anionic or capable of forming an anion can be inorganic or organic.

Examples of inorganic anion part of the salt include but are not limited to chloride, bromide, iodide, fluoride, sulphate, phosphate, nitrate, nitrite, hydrogen carbonates and hydrogen sulphate.

Examples of organic anion part of the salt include but are not limited to formate, alkanoates, carbonates, acetates, trifluoroacetate, trichloroacetate, propionate, glycolate, thiocyanate, lactate, succinate, malate, citrates, benzoates, cinnamates, oxalates, alkylsulphates, alkylsulphonates, arylsulphonates aryldisulphonates, alkylphosphonates, arylphosphonates, aryldiphosphonates, p-toluenesulphonate, and salicylate.

Examples of inorganic cation part of the salt include but are not limited to alkali and alkaline earth metals.

Examples of organic cation part of the salt include but are not limited to pyridine, methyl amine, imidazole, benzimidazole, histidine, phosphazene, tetramethyl ammonium, tetrabutyl ammonium, choline and trimethyl amine.

Metal ions in metal complexes of the compound of Formula I are especially the ions of the elements of the second main group, especially calcium and magnesium, of the third and fourth main group, especially aluminium, tin and lead, and also of the first to eighth transition groups, especially chromium, manganese, iron, cobalt, nickel, copper, zinc and others. Particular preference is given to the metal ions of the elements of the fourth period and the first to eighth transition groups. Here, the metals can be present in the various valencies that they can assume.

Compounds of the present invention may exist in more than one form, and thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of the present invention can exhibit beneficial effects (e.g., suitability for preparation of useful formulations, improved biological performance) relative to another polymorph or a mixture of polymorphs of the same compound of the present invention. Preparation and isolation of a particular polymorph of a compound of the present invention can be achieved by methods known to those skilled in the art including, for example, crystallization using selected solvents and temperatures.

The present invention also relates to a process for preparing the compound of Formula I. The process comprises reacting a compound of Formula I with a compound of Formula IN optionally using a suitable base and a suitable solvent. The reaction is carried out at a temperature ranging from 20° C. to 150° C. The reaction is depicted herein below:

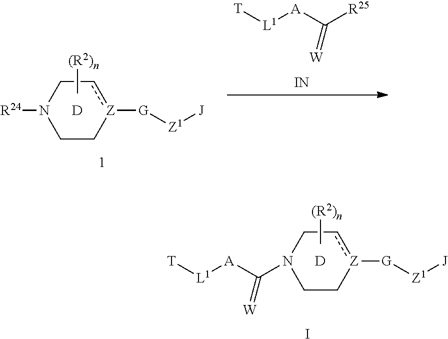

wherein, R²⁴ is hydrogen, or —OC(═O)C₁-C₆-alkyl; R²⁵ is hydroxy, chlorine, or —OC₁-C₆-alkyl; and R², A, G, J, L¹, T, W, Z¹ and n are each as defined herein above.

Alternatively, the compound of Formula 2 is reacted win IN to obtain I in the presence of a suitable base, a suitable solvent at suitable temperature conditions.

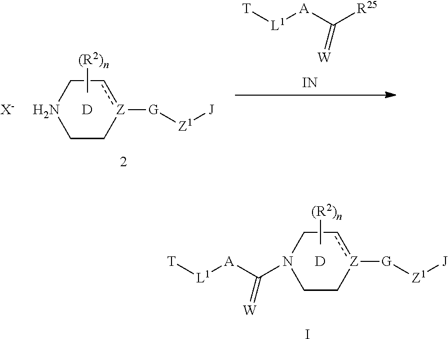

wherein, X⁻ is selected from HSO₄⁻, Cl⁻, Br⁻, I⁻, CH₃C(═O)O⁻, CF₃C(═O)O⁻; R²⁵ is hydroxy, chlorine, or —OC₁-C₆-alkyl; L¹ is O or S; and R², A, G, J, T, W, Z¹ and n are each as defined herein above.

The present invention also relates yet another process for preparing the compound of Formula I, wherein L¹ is N. In the first step of the process the compound of Formula 4 is prepared by reacting the compound of Formula 2 or 3 with the compound of Formula IN′. The reaction is depicted herein below:

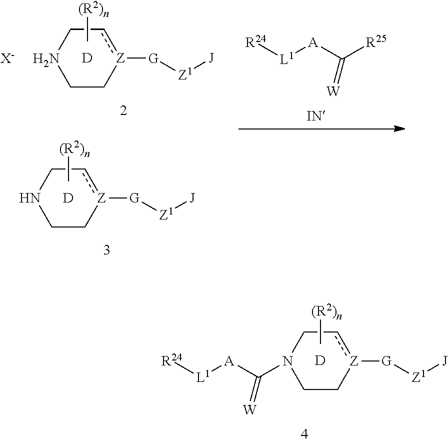

wherein, L¹ is N; R², R²⁴, R²⁵, A, G, J, W, X, Z¹ and n are each as defined herein above.

The compound of Formula 4, wherein R²⁴ is —OC(═O)C₁-C₆-alkyl is converted into the compound of Formula 4, wherein R²⁴ is hydrogen, by the process known in the literature.

In the second step of the process, the compound of Formula 4, wherein R²⁴ is hydrogen, is reacted with the compound of Formula IN″ to obtain the compound of Formula I. The reaction is depicted herein below:

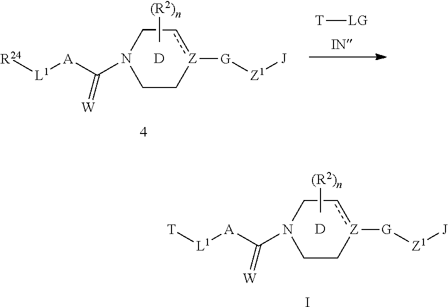

wherein, L¹ is N, LG is leaving group such as halogen; R², R²⁴, A, G, J, T, W, Z, Z¹ and n are each as defined herein above.

The present invention also relates to a novel compound of Formula 4 which useful in the synthesis of Formula I:

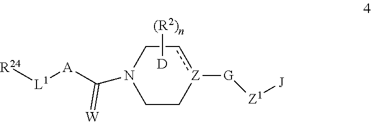

wherein, L¹ is N; R², R²⁴, A, G, J, W, Z, Z¹ and n are each as defined herein above.

The present invention also relates a composition comprising the compound of Formula I and one or more excipient.

The compound of Formula I of the present invention in the composition can be an agriculturally acceptable salt, metal complex, constitutional isomer, stereo-isomer, diastereoisomer, enantiomer, chiral isomer, atropisomer, conformer, rotamer, tautomer, optical isomer, geometric isomer, polymorph, or N-oxide thereof.

The excipient may be an inert carrier or any other essential ingredient such as surfactants, additives, solid diluents and liquid diluents.

The composition of the present invention may additionally comprise at least one active compatible compound selected from fungicides, insecticides, nematicides, acaricides, biopesticides, herbicides, plant growth regulators, antibiotics, fertilisers and nutrients. The compounds used in the composition and in combination with the compound of Formula I are also termed as active compatible compounds.

The concentration of the compound of Formula I in the composition of the present invention ranges from 1 to 90% by weight with respect to the total weight of the composition, preferably from 5 to 50% by weight with respect to the total weight of the composition.

The known and reported active compounds such as fungicides, insecticides, nematicides, acaricides, biopesticides, herbicides, plant growth regulators, antibiotics and nutrients can be combined with at least one compound of Formula I of the present invention. For example, fungicides, insecticides, nematicides, acaricides, biopesticides, herbicides, plant growth regulators, antibiotics, fertilizers and nutrients disclosed and reported in WO201776739 (A to O) can be combined with compound of Formula I of the present invention. The present invention also relates to such combinations comprising the compound of the present invention and active compatible compounds reported in WO201776739.

The fungicides, insecticides, nematicides, acaricides, biopesticides, herbicides, plant growth regulators, antibiotics, fertilizers and nutrients reported in WO201776739, are not reproduced herein for the sake of brevity and are incorporated herein by way of reference as non-limiting examples to be combined with at least one compound of Formula I of the present invention.

The present invention also relates to a use of the compound of Formula I or the combination comprising the compound of Formula I or the composition comprising the compound of Formula I for controlling or preventing phytopathogenic micro-organisms such as fungi, stramenopiles, bacteria, insects, nematodes, trematodes, and mites in agricultural crops and or horticultural crops.

Particularly, the present invention also relates to a use of the compound of Formula I or the combination or the composition for controlling or preventing phytopathogenic micro-organisms in agricultural crops and or horticulture crops.

The compound of Formula I or the combination or the composition of the present invention may be used to treat several fungal pathogens. Non-limiting examples of pathogens of fungal diseases which can be treated in accordance with the invention include:

Diseases caused by pathogens from the group of the Stramenopiles, particularly by Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum; Diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator; Erysiphe species, for example Erysiphe cichoracearu; Diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus; Leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidial form: Drechslera, syn: Helminthosporium) or Cochliobolus miyabeanus; Colletotrichum species, for example Colletorichum lindemulhanium; Cycloconium species, for example Cycloconium oleaginum; Diaporthe species, for example Diaporthe cirri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans; Magnaporthe species, for example Magnaporthe grisea; Microdochium species, for example Microdochium nivale; Mycosphaerella species, for example Mycosphaerella graminicola, Mycosphaerella arachidicola or Mycosphaerella fijiensis; Phaeosphaeria species, for example Phaeosphaeria nodorum; Pyrenophora species, for example Pyrenophora teres or Pyrenophora tritici repentis; Ramularia species, for example Ramularia collo-cygni or Ramularia areola; Rhynchosporium species, for example Rhynchosporium secalis; Septoria species, for example Septoria apii or Septoria lycopersici; Stagonospora species, for example Stagonospora nodorum; Typhula species, for example Typhula incarnata; Venturia species, for example Venturia inaequalis;

Root and stem diseases caused, for example, by Corticium species, for example Corticium graminearum; Fusarium species, for example Fusarium oxysporum; Gaeumannomyces species, for example Gaeumannomyces graminis; Plasmodiophora species, for example Plasmodiophora brassicae; Rhizoctonia species, for example Rhizoctonia solani; Sarocladium species, for example Sarocladium oryzae; Sclerotium species, for example Scilerotium oryzae; Tapesia species, for example Tapesia acuformis; Thielaviopsis species, for example Thielaviopsis basicola; Ganoderma species, for example Ganoderma lucidum;

Ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Stagnospora species, for example Stagnospora nodorum;

Diseases caused by smut fungi, for example Sphacelotheca species, for example Sphacelotheca reiliana; Tilletia species, for example Tilletia caries or Tilletia controversa; Urocystis species, for example Urocystis occulta; Ustilago species, for example Ustilago nuda;

Fruit rot caused, for example, by Aspergillus species, for example Aspergillus flavus; Botrylis species, for example Botrytis cinerea; Penicillium species, for example Penicillium expansum or Penicillium purpurogenum; Rhizopus species, for example Rhizopus stolonifer; Sclerotinia species, for example Sclerotinia sclerotiorum; Verticilium species, for example Verticilium alboatrum;

Seed- and soil-borne rot and wilt diseases, and also diseases of seedlings, caused, for example, by Alternaria species, for example Alternaria brassicicola; Aphanomyces species, for example Aphanomyces euteiches; Ascochyta species, for example Ascochyta lentis; Aspergillus species, for example Aspergillus flavus; Cladosporium species, for example Cladosporium herbarum; Cochliobolus species, for example Cochliobolus salivus (conidial form: Drechslera, Bipolaris Syn: Helminthosporium); Colletotrichum species, for example Colletotrichum coccodes; Fusarium species, for example Fusarium culmorum; Gibberella species, for example Gibberella zeae; Macrophomina species, for example Macrophomina phaseolina; Microdochium species, for example Microdochium nivale; Monographella species, for example Monographella nivalis; Penicillium species, for example Penicillium expansum; Phoma species, for example Phoma lingam; Phomopsis species, for example Phomopsis sojae; Phytophihora species, for example Phytophihora cactorum; Pyrenophora species, for example Pyrenophora graminea; Pyricularia species, for example Pyricularia oryzae; Pythium species, for example Pythium ultimum; Rhizoctonia species, for example Rhizoctonia solani; Rhizopus species, for example Rhizopus oryzae; Sclerotium species, for example Sclerotium rolfsii; Septoria species, for example Septoria nodorum; Typhula species, for example Typhula incarnata; Verticillium species, for example Verticillium dahliae;

Cancers, galls and witches' broom caused, for example, by Nectria species, for example Nectria galligena;

Wilt diseases caused, for example, by Monilinia species, for example Monilinia laxa;

Deformations of leaves, flowers and fruits caused, for example, by Exobasidium species, for example Exobasidium vexans; Taphrina species, for example Taphrina deformans;

Degenerative diseases in woody plants, caused, for example, by Esca species, for example Phaeomoniella chlamydospora, Phaeoacremonium aleophilum or Fomitiporia mediterranea; Ganoderma species, for example Ganoderma boninense;

Diseases of flowers and seeds caused, for example, by Botrytis species, for example Botrytis cinerea;

Diseases of plant tubers caused, for example, by Rhizoctonia species, for example Rhizoctonia solani; Helminthosporium species, for example Helminthosporium solani;

Diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora; Ralstonia species, for example Ralsionia solanacearum;

Fungal diseases on roots and the stem base caused, for example, by black root rot (Calonectria crotalariae), charcoal rot (Macrophomina phaseolina), fusarium blight or wilt, root rot, and pod and collar rot (Fusariwn oxysporum, Fusariwn orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythiwn irregulare, Pythiwn debaryanum, Pythium myriotylwn, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

Plants which can be treated in accordance with the invention include the following: Rosaceae sp (for example pome fruits such as apples, pears, apricots, cherries, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Vitaceae sp. (for example grapes); Solanaceae sp. (for example tomatoes, peppers), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Poaceae/Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet; fodder beet, swiss chard, beetroot); Malvaceae (for example cotton); useful plants and ornamental plants for gardens and wooded areas; and genetically modified varieties of each of these plants.

The agricultural or horticulture crops are wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Particularly, the agriculture or horticulture crops are cereals, corn, rice, soybean and other leguminous plants, fruits and fruit trees, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers, other vegetables and ornamentals.

The present invention further relates to the use of the compound of Formula I or the combination comprising the compound of Formula I or the composition comprising the compound of Formula I for treating seeds with the purpose of protecting the seeds, the germinating plants and emerged seedlings against phytopathogenic micro-organisms.

The present invention further relates to seeds which have been treated with the compound of Formula I or the combination comprising the compound of Formula I or the composition comprising the compound of Formula I for protection from phytopathogenic micro-organisms.

The present invention also relates to a method of controlling or preventing infestation of useful plants by phytopathogenic micro-organisms in agricultural crops and or horticultural crops wherein the compound of Formula I or the combination comprising the compound of Formula I or the composition comprising the compound of Formula I, is applied to the plants, to parts thereof or the locus thereof. The effective amount of compound of Formula I ranges from 1 to 5000 gai per hectare.

Also, the present invention relates to the compound of Formula I or the combination comprising the compound of Formula I or the composition comprising the compound of Formula I applied to a plant, plant parts or locus thereof.

The present invention furthermore includes a method for treating seed, particularly seeds (dormant, primed, pregerminated or even with emerged roots and leaves) treated with the compound of Formula I or the combination comprising the compound of Formula I or the composition comprising the compound of Formula I. In these methods, the compound of Formula I or the combination comprising the compound of Formula I or the composition comprising the compound of Formula I is applied to the seeds of plants for controlling or preventing infestation of useful plants by phytopathogenic micro-organisms in agricultural and or horticultural corps.

It is also desirable to optimize the amount of the active ingredient used so as to provide the best possible protection for the plants, the plant parts, or the seeds, the germinating plants and emerged seedlings from attack by phytopathogenic micro-organisms, but without damaging the plants themselves by the active ingredient used. In particular, methods for the treatment of seed should also take into consideration the intrinsic phenotypes of transgenic plants in order to achieve optimum protection of the seed and the germinating plant with a minimum of crop protection compositions being employed.

One of the advantages of the present invention is that the treatment of the seeds with the compound of Formula I or the combination comprising the compound of Formula I or the composition comprising the compound of Formula I not only protects the seed itself, but also the resulting plants after emergence, from animal pests and/or phytopathogenic harmful micro-organisms. In this way, the immediate treatment of the crop at the time of sowing or shortly thereafter protect plants as well as seed treatment in prior to sowing. It is likewise considered to be advantageous that the compound of Formula I or the combination comprising the compound of Formula I or the composition comprising the compound of Formula I can be used especially also for transgenic seed, in which case the plant which grows from this seed is capable of expressing a protein which acts against pests, herbicidal damage or abiotic stress. The treatment of such seeds with the compound of Formula I or the combination comprising the compound of Formula I or the composition comprising the compound of Formula I, for example an insecticidal protein, can result in control of certain pests. Surprisingly, a further enhanced effect can be observed in this case, which additionally increases the effectiveness for protection against attack by pests, micro-organisms, weeds or abiotic stress.

The compound of Formula I or the combination comprising the compound of Formula I or the composition comprising the compound of Formula I is suitable for protection of seed of any plant variety which is used in agriculture, in the greenhouse, in forests or in horticulture. More particularly, the seed is that of cereals (such as wheat, barley, rye, millet and oats), oilseed rape, maize, cotton, soybeen, rice, potatoes, sunflower, beans, coffee, beet (e.g. sugar beet and fodder beet), peanut, vegetables (such as tomato, cucumber, onions and lettuce), lawns and ornamental plants. Of particular significance is the treatment of the seed of wheat, soybean, oilseed rape, maize and rice.

As also described below, the treatment of transgenic seed with the compound of Formula I or the combination comprising the compound of Formula I or the composition comprising the compound of Formula I, is of particular significance. This refers to the seed of plants containing at least one heterologous gene which allows the expression of a polypeptide or protein, e.g. having insecticidal properties. These heterologous genes in transgenic seeds may originate, for example, from micro-organisms of the species of Bacillus, Rhizobium, Pseudomonas, Serratia, Trichoderma, Clavibacter, Glomus or Gliocladium. These heterologous genes preferably originate from Bacillus sp., in which case the gene product is effective against the European corn borer and/or the Western corn rootworm. Particularly preferably, the heterologous genes originate from Bacillus thuringiensis.

In the context of the present invention, the compound of Formula I or the combination comprising the compound of Formula I or the composition comprising the compound of Formula I is applied to seeds. Particularly, the seed is treated in a state in which it is sufficiently stable for no damage to occur in the course of treatment. In general, seeds can be treated at any time between harvest and some time after sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again, or seeds just after priming, or seeds stored in primed conditions or pre-germinated seeds, or seeds sown on nursery trays, tapes or paper.

When treating the seeds, it generally has to be ensured that the amount of the compound of Formula I or the combination comprising the compound of Formula I or the composition comprising the compound of Formula I applied to the seed and/or the amount of further additives is selected such that the germination of the seed is not impaired, or that the resulting plant is not damaged.

The compound of Formula I or the combination comprising the compound of Formula I or the composition comprising the compound of Formula I can be applied directly, i.e. without containing any other components and without having been diluted. In general, it is preferable to apply the compositions comprising compounds of Formula I to the seed in the form of a suitable formulation. Suitable formulations and methods for seed treatment are known to those skilled in the art. The compound of Formula I can be converted to the customary formulations relevant to on-seed applications, such as solutions, emulsions, suspensions, powders, foams, slurries or combined with other coating compositions for seed, such as film forming materials, pelleting materials, fine iron or other metal powders, granules, coating material for inactivated seeds, and also ULV Formulations.

In the treatment of seeds to facilitate plantability seeds can be coated with polymer. The polymer coating is comprised of a binder, a wax and a pigment, and one or more stabilizers in an amount effective to stabilize the suspension. The binder can be a polymer selected from the group comprising of vinyl acetate-ethylene copolymer, vinyl acetate homopolymer, vinyl acetate-acrylic copolymer, vinylacrylic, acrylic, ethylene-vinyl chloride, vinyl ether maleic anhydride, or butadiene styrene. Other similar polymers can be used.

These formulations are prepared in a known manner, by mixing the active ingredients or active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing Formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of active agrochemical ingredients. Usable with preference are alkylnaphthalenesulphonates, such as diisopropyl- or diisobutylnaphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Useful nonionic dispersants include especially ethylene oxide/propylene oxide block polymers, alkylphenol polyglycol ethers and tristryrylphenol polyglycol ether, and the phosphated or sulphated derivatives thereof. Suitable anionic dispersants are especially lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Silicone antifoams and magnesium stearate can be used with preference.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances usable for such purposes in agrochemical compositions. Preferred examples include cellulose derivatives, acrylic acid derivatives, xanthan, modified clays and finely divided silica.

Adhesives which may be present in the seed dressing formulations usable in accordance with the invention are all customary binders usable in seed dressing products. Preferred examples include polyvinylpyrrolidone, polyvinyl acetate, polyvinyl alcohol and tylosc.

The formulations for on-seed applications usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also seeds of maize, soybean, rice, oilseed rape, peas, beans, cotton, sunflowers, and beets, or else a wide variety of different vegetable seeds. The formulations usable in accordance with the invention, or the dilute preparations thereof, can also be used for seeds of transgenic plants. In this case, enhanced effects may also occur in interaction with the substances formed by expression.

For treatment of seeds with the formulations usable in accordance with the invention, or the preparations prepared therefrom by adding water, all mixing units usable customarily for on-seed applications are useful. Specifically, the procedure in on-seed applications is to place the seeds into a mixer, to add the particular desired amount of the formulations, either as such or after prior dilution with water, and to mix everything until all applied formulations are distributed homogeneously on the seeds. If appropriate, this is followed by a drying operation.

The application rate of the formulations usable in accordance with the invention can be varied within a relatively wide range. It is guided by the particular content of the active ingredients in the formulations and by the seeds. The application rates of each single active ingredient are generally between 0.001 and 15 gai per kilogram of seed, preferably between 0.01 and 5 gai per kilogram of seed.

When using the compound of Formula I as fungicides, the application rates can be varied within a relatively wide range, depending on the kind of application. The application rate of the compound of Formula I or the combination comprising the compound of Formula I or the composition comprising the compound of Formula I, is:

in the case of treatment of plant parts, for example leaves: from 0.1 to 10000 gai/ha, preferably from 5 to 1000 gai/ha, more preferably from 5 to 100 gai/ha (in the case of application by watering or dripping, it is even possible to reduce the application rate, especially when inert substrates such as rockwool or perlite are used);

in the case of seed treatment: from 0.1 to 200 gai per 100 kg of seed, preferably from 1 to 150 gai per 100 kg of seed, more preferably from 2.5 to 25 gai per 100 kg of seed.

in the case of soil treatment: from 0.1 to 10000 gai/ha, preferably from 1 to 1000 gai/ha.

These application rates are merely by way of example and are not limiting for the purposes of the invention.

In some cases, the compound of Formula I may, at particular concentrations or application rates, also be used as safeners, growth regulators or agents to improve plant properties, or as microbicides, for example as fungicides, antimycotics, bactericides, viricides (including compositions against viroids) or as compositions against phytoplasmas MLO (Mycoplasma-like organisms) and RLO (Rickettsia-like organisms).

The compound of Formula I may intervene in physiological processes of plants and can therefore also be used as plant growth regulators. Plant growth regulators may exert various effects on plants. The effect of the substances depends essentially on the time of application in relation to the developmental stage of the plant, the plant variety and also on the amounts of active ingredient applied to the plants or their environment and on the type of application. In each case, growth regulators should have a particular desired effect on the crop plants.

Growth regulating effects, comprise earlier germination, better emergence, more developed root system and/or improved root growth, increased ability of tillering, more productive tillers, earlier flowering, increased plant height and/or biomass, shorting of stems, improvements in shoot growth, number of kernels/ear, number of ears/m², number of stolons and/or number of flowers, enhanced harvest index, bigger leaves, less dead basal leaves, improved phyllotaxy, earlier maturation/earlier fruit finish, homogenous riping, increased duration of grain filling, better fruit finish, bigger fruit/vegetable size, sprouting resistance and reduced lodging.

Increased or improved yield is referring to total biomass per hectare, yield per hectare, kernel/fruit weight, seed size and/or hectolitre weight as well as to improved product quality, comprising: improved processability relating to size distribution (kernel, fruit, etc.), homogenous riping, grain moisture, better milling, better vinification, better brewing, increased juice yield, harvestability, digestibility, sedimentation value, falling number, pod stability, storage stability, improved fiber length/strength/uniformity, increase of milk and/or meet quality of silage fed animals, adaptation to cooking and frying;

further comprising improved marketability relating to improved fruit/grain quality, size distribution (kernel, fruit, etc.), increased storage/shelf-life, firmness/softness, taste (aroma, texture, etc.), grade (size, shape, number of berries, etc.), number of berries/fruits per bunch, crispness, freshness, coverage with wax, frequency of physiological disorders, colour, etc.;

further comprising increased desired ingredients such as e.g. protein content, fatty acids, oil content, oil quality, aminoacid composition, sugar content, acid content (pH), sugar/acid ratio (Brix), polyphenols, starch content, nutritional quality, gluten content/index, energy content, taste, etc.;

and further comprising decreased undesired ingredients such as e.g. less mycotoxines, less aflatoxines, geosmin level, phenolic aromas, lacchase, polyphenol oxidases and peroxidases, nitrate content etc.

Plant growth-regulating compounds can be used, for example, to slow down the vegetative growth of the plants. Such growth depression is of economic interest, for example, in the case of grasses, since it is thus possible to reduce the frequency of grass cutting in ornamental gardens, parks and sport facilities, on roadsides, at airports or in fruit crops. Also of significance is the inhibition of the growth of herbaceous and woody plants on roadsides and in the vicinity of pipelines or overhead cables, or quite generally in areas where vigorous plant growth is unwanted.

Also important is the use of growth regulators for inhibition of the longitudinal growth of cereal. This reduces or completely eliminates the risk of lodging of the plants prior to harvest. In addition, growth regulators in the case of cereals can strengthen the culm, which also counteracts lodging. The employment of growth regulators for shortening and strengthening culms allows the deployment of higher fertilizer volumes to increase the yield, without any risk of lodging of the cereal crop.

In many crop plants, vegetative growth depression allows denser planting, and it is thus possible to achieve higher yields based on the soil surface. Another advantage of the smaller plants obtained in this way is that the crop is easier to cultivate and harvest.

Reduction of the vegetative plant growth may also lead to increased or improved yields because the nutrients and assimilates are of more benefit to flower and fruit formation than to the vegetative parts of the plants.

Alternatively, growth regulators can also be used to promote vegetative growth. This is of great benefit when harvesting the vegetative plant parts. However, promoting vegetative growth may also promote generative growth in that more assimilates are formed, resulting in more or larger fruits.

Furthermore, beneficial effects on growth or yield can be achieved through improved nutrient use efficiency, especially nitrogen (N)-use efficiency, phosphours (P)-use efficiency, water use efficiency, improved transpiration, respiration and/or CO₂ assimilation rate, better nodulation, improved Ca-metabolism etc.

Likewise, growth regulators can be used to alter the composition of the plants, which in turn may result in an improvement in quality of the harvested products. Under the influence of growth regulators, parthenocarpic fruits may be formed. In addition, it is possible to influence the sex of the flowers. It is also possible to produce sterile pollen, which is of great importance in the breeding and production of hybrid seed.

Use of growth regulators can control the branching of the plants. On the one hand, by breaking apical dominance, it is possible to promote the development of side shoots, which may be highly desirable particularly in the cultivation of ornamental plants, also in combination with an inhibition of growth. On the other hand, however, it is also possible to inhibit the growth of the side shoots. This effect is of particular interest, for example, in the cultivation of tobacco or in the cultivation of tomatoes.

Under the influence of growth regulators, the amount of leaves on the plants can be controlled such that defoliation of the plants is achieved at a desired time. Such defoliation plays a major role in the mechanical harvesting of cotton, but is also of interest for facilitating harvesting in other crops, for example in viticulture.

Defoliation of the plants can also be undertaken to lower the transpiration of the plants before they are transplanted.

Furthermore, growth regulators can modulate plant senescence, which may result in prolonged green leaf area duration, a longer grain filling phase, improved yield quality, etc.

Growth regulators can likewise be used to regulate fruit dehiscence. On the one hand, it is possible to prevent premature fruit dehiscence. On the other hand, it is also possible to promote fruit dehiscence or even flower abortion to achieve a desired mass (“thinning”). In addition, it is possible to use growth regulators at the time of harvest to reduce the forces required to detach the fruits, in order to allow mechanical harvesting or to facilitate manual harvesting.

Growth regulators can also be used to achieve faster or else delayed ripening of the harvested material before or after harvest. This is particularly advantageous as it allows optimal adjustment to the requirements of the market. Moreover, growth regulators in some cases can improve the fruit colour. In addition, growth regulators can also be used to synchronize maturation within a certain period of time. This establishes the prerequisites for complete mechanical or manual harvesting in a single operation, for example in the case of tobacco, tomatoes or coffee.

By using growth regulators, it is additionally possible to influence the resting of seed or buds of the plants, such that plants such as pineapple or ornamental plants in nurseries, for example, germinate, sprout or flower at a time when they are normally not inclined to do so. In areas where there is a risk of frost, it may be desirable to delay budding or germination of seeds with the aid of growth regulators, in order to avoid damage resulting from late frosts.

Finally, growth regulators can induce resistance of the plants to frost, drought or high salinity of the soil. This allows the cultivation of plants in regions which are normally unsuitable for this purpose.

The compound of Formula I or the combination comprising the compound of Formula I or the composition comprising the compound of Formula I also exhibit potent strengthening effect in plants.

Accordingly, they can be used for mobilizing the defences of the plant against attack by undesirable micro-organisms.

Plant-strengthening (resistance-inducing) substances in the present context are substances capable of stimulating the defence system of plants in such a way that the treated plants, when subsequently inoculated with undesirable micro-organisms, develop a high degree of resistance to these micro-organisms.

Further, in context with the present invention plant physiology effects comprise the following: Abiotic stress tolerance, comprising tolerance to high or low temperatures, drought tolerance and recovery after drought stress, water use efficiency (correlating to reduced water consumption), flood tolerance, ozone stress and UV tolerance, tolerance towards chemicals like heavy metals, salts, pesticides etc.

Biotic stress tolerance comprising increased fungal resistance and increased resistance against nematodes, viruses and bacteria. In context with the present invention, biotic stress tolerance preferably comprises increased fungal resistance and increased resistance against nematodes.

Increased plant vigor, comprising plant health/plant quality and seed vigor, reduced stand failure, improved appearance, increased recovery after periods of stress, improved pigmentation (e.g. chlorophyll content, stay-green effects, etc.) and improved photosynthetic efficiency.

In addition, the compound of Formula I or the combination comprising the compound of Formula I or the composition comprising the compound of Formula I can reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asialicum, F avenaceum, F. crookwellense, F. culmorum, F. graminearum (Gibberella zeae), F. equiseti, F. fujikoroi, F. musarum, F. oxysporum, F. proliferatum, F. poae, F. pseudograminearwn, F. sambucinum, F. scirpi, F. semitectum, F. solani, F. sporotrichoides, F. langsethiae, F. subglutinans, F. tricinclum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. claviforme, P. roqueforti, Claviceps spec., such as C. purpurea, C. fusiformis, C. paspali, C. africana, Stachybotrys spec. and others.

The compound of Formula I or the combination comprising the compound of Formula I or the composition comprising the compound of Formula I can also be used in the protection of materials, for protection of industrial materials against attack and destruction by phytopathogenic micro-organisms.

In addition, the compound of Formula I or the combination comprising the compound of Formula I or the composition comprising the compound of Formula I can be used as antifouling compositions, alone or in combinations with other active ingredients.

Industrial materials in the present context are understood to mean inanimate materials which have been prepared for use in industry. For example, industrial materials which are to be protected by inventive compositions from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by micro-organisms. Parts of production plants and buildings, for example cooling-water circuits, cooling and heating systems and ventilation and air-conditioning units, which may be impaired by the proliferation of micro-organisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compound of Formula I or the combination comprising the compound of Formula I or the composition comprising the compound of Formula I may prevent adverse effects, such as rotting, decay, discoloration, decoloration or formation of mould.

In the case of treatment of wood the compound of Formula I or the compound of Formula I in the composition optionally comprising at least one active compatible compound may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting a composition according to the invention; this includes for example direct application, spraying, dipping, injection or any other suitable means.

In addition, the compound of Formula I or the combination comprising the compound of Formula I or the composition comprising the compound of Formula I can be used to protect objects which come into contact with saltwater or brackish water, especially hulls, screens, nets, buildings, moorings and signalling systems, from fouling.

The compound of Formula I or the combination comprising the compound of Formula I or the composition comprising the compound of Formula I can also be employed for protecting storage goods.

Storage goods are understood to mean natural substances of vegetable or animal origin or processed products thereof which are of natural origin, and for which long-term protection is desired.

Storage goods of vegetable origin, for example plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The compound of Formula I or the combination comprising the compound of Formula I or the composition comprising the compound of Formula I may prevent adverse effects, such as rotting, decay, discoloration, decoloration or formation of mould.

Micro-organisms capable of degrading or altering the industrial materials include, for example, bacteria, fungi, yeasts, algae and slime organisms. The compound of Formula I or the combination comprising the compound of Formula I or the composition comprising the compound of Formula I preferably act against fungi, especially moulds, wood-discoloring and wood-destroying fungi (Ascomycetes, Basidiomycetes, Deuteromycetes and Zygomycetes), and against slime organisms and algae. Examples include micro-organisms of the following genera: Alternaria, such as Alternaria tenuis; Aspergillus, such as Aspergillus niger, Chaetomium, such as Chaetomium globosum; Coniophora, such as Coniophora puetana, Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

In addition, the compound of Formula I or the combination comprising the compound of Formula I or the composition comprising the compound of Formula I also has very good antimycotic effects. They have a very broad antimycotic activity spectrum, especially against dermatophytes and yeasts, moulds and diphasic fungi (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The enumeration of these fungi by no means constitutes a restriction of the mycotic spectrum covered, and is merely of illustrative character.

The compound of Formula I or the combination comprising the compound of Formula I or the composition comprising the compound of Formula I can be used also to control important fungal pathogens in fish and crustacea farming, e.g. saprolegnia diclina in trouts, saprolegnia parasitica in crayfish.

The compound of Formula I or the combination comprising the compound of Formula I or the composition comprising the compound of Formula I can therefore be used both in medical and in non-medical applications.

The compound of Formula I or the combination comprising the compound of Formula I or the composition comprising the compound of Formula I can be used as such, in the form of their formulations or the use forms prepared therefrom, such as ready-to-use solutions, suspensions, wettable powders, pastes, soluble powders, dusts and granules. Application is accomplished in a customary manner, for example by watering, spraying, atomizing, broadcasting, dusting, foaming, spreading-on and the like. It is also possible to deploy the active ingredients by the ultra-low volume method or to inject the active ingredient preparation/the active ingredient itself into the soil. It is also possible to treat the seed of the plants.

It is possible to treat all plants and their parts in accordance with the invention, preferably with wild plant species and plant cultivars, or those obtained by conventional biological breeding methods, such as crossing or protoplast fusion, and also parts thereof. In a further preferred embodiment, transgenic plants and plant cultivars obtained by genetic engineering methods, if appropriate in combination with conventional methods (Genetically Modified Organisms), and parts thereof are treated. The terms “parts” or “parts of plants” or “plant parts” have been explained above. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance.

Plants or plant cultivars (obtained by plant biotechnology methods such as genetic engineering) which may also be treated according to the invention show altered quantity, quality and/or storage-stability of the harvested product and/or altered properties of specific ingredients of the harvested product.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics.

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants or plant cultivars (that can be obtained by plant biotechnology methods such as genetic engineering) which may also be treated according to the invention are plants, such as tobacco plants, with altered post-translational protein modification patterns.

The invention disclosed in the present invention shall now be elaborated with the help of non-limiting schemes and examples.

### CHEMISTRY EXAMPLES

The definitions of T, L¹, A, W, Z, G, Z¹, J, n and R² in the schemes below are as defined above in the description unless otherwise noted.

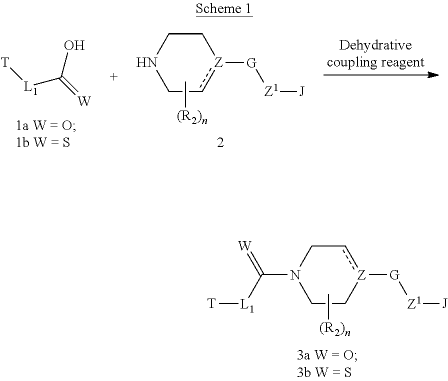

The compounds of Formulae 3a and 3b can be prepared by one or more of the following methods and variations as described in Schemes 1-11.

As shown in Scheme 1, a compound of Formula 3a is prepared by the process which involves coupling of an acid of Formula 1a with an amine of Formula 2 (or its salt) in the presence of a dehydrative coupling reagent such as dicyclohexylcarbodiimide (DCC), 1-(3-dimethylaminopropyl)-3-ethylcarbodiimide hydrochloride (EDC), O-benzotriazol-1-yl-tetramethyluronium hexafluoro-phosphate (HBTU), or 1-[Bis(dimethylamino)methylene]-1H-1,2,3-triazolo[4,5-b]pyridinium 3-oxid hexafluorophosphate (HATU). Polymer-supported reagents such as polymer-bound cyclohexylcarbodiimide may also be used. The reactions are typically carried out at 0-40° C. in a solvent such as dichloromethane, acetonitrile or dimethylformamide in the presence of a base such as triethylamine or diisopropylethylamine.

Alternatively, the compound of Formula 3b can be prepared by reacting the compound of Formula 3a with a variety of standard thiating reagents such as phosphorus pentasulfide or 2, 4-bis (4-methoxyphenyl)-1, 3-dithia-2, 4-diphosphetane-2, 4-disulfide (Lawesson's reagent).

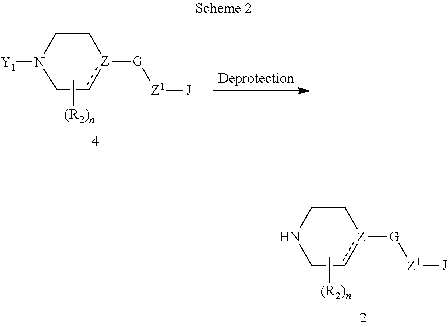

The compound of Formula 2 can be prepared from a compound of Formula 4 wherein Y₁ is an amine-protecting group as shown in Scheme 2.

The compound of the Formula 4 is converted into the compound of the Formula 2 by suitable methods for removing protecting groups described in the literature (Protective Groups in Organic Synthesis”; Theodora W. Greene, Peter G. M. Wuts; Wiley-Interscience; Third Edition: 1999; 494-653).

For example, tert-Butoxycarbonyl and benzyloxycarbonyl protecting groups can be removed in an acidic medium (for example with hydrochloric acid or trifluoroacetic acid). Acetyl protecting groups can be removed under basic conditions (for example with potassium carbonate or cesium carbonate). Benzylic protecting groups can be removed hydrogenolytically with hydrogen in the presence of a catalyst (for example palladium on activated carbon).

After the reaction is completed, the compound of Formula 2 are separated from the reaction mixture by one of the customary separation techniques. If necessary, the compound is purified by recrystallization or chromatography, or can, if desired, also be used in the next step without prior purification. It is also possible to isolate the compound of the Formula 2 as a salt, for example as a salt of hydrochloric acid or of trifluoroacetic acid.

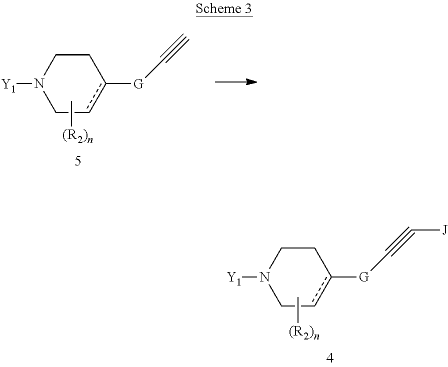

As shown in the scheme 3, syntheses of a compound of Formula 4 involves palladium-catalyzed cross-coupling reaction of terminal alkynes of a compound of Formula 5 with organic electrophiles such as alkyl bromides or chlorides. The most widely used of these is a cross between the Cu-promoted Castro-Stephens reaction and the Heck alkynylation, known as the Sonogashira reaction. The compound of Formula 4 can also be obtained using palladium-based systems to catalyze the reaction of aryl halides and terminal alkynes. For references, see for example Metal-Catalyzed Cross-Coupling Reactions, Vol. 2; de Meijere, A.; Diederich, F., Eds.; Wiley-VCH: Weinheim, 2004., Negishi, E.; Anastasia, L. Chem. Rev. 2003, 103, 1979, Castro, C. E.; Stephens, R. D. J. Org. Chem. 1963, 28, 2163, Dieck, H. A.; Heck, F. R. J. Organomet. Chem. 1975, 93, 259, Sonogashira, K. J. Organomet. Chem. 2002, 653, 46.

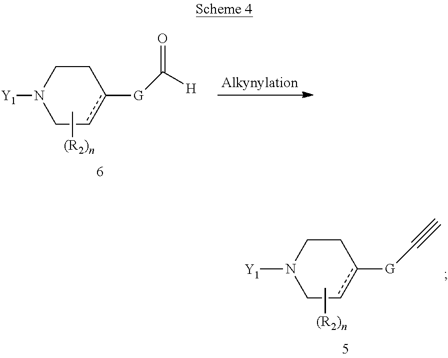

The compound of Formula 5 can be prepared from a compound of Formula 6 as shown in Scheme 4.

In the literature, it is known that alkynylation of aldehydes can be achieved by Corey-Fuchs reaction (Tetrahedron Lett, 1972, 36, 3769) or a Seyferth-Gilbert homologization (see, for example, J. Org. Chem., 1996, 61, 2540). Alternatively, the compound of Formula 5 can also be prepared from the compound of Formula 6 with Bestmann-Ohira's reagent analogously to the literature instructions (see, for example, Synthesis, 2004, 59). Alkynylation with Bestmann-Ohira's reagent in methanol or ethanol is preferably used in equivalent of potassium carbonate or sodium carbonate.

The compound of Formula 6 and the alkynylation reagent are used in equimolar amounts, but the Bestmann-Ohira's reagent can be used in excess if necessary. The reaction is preferably carried out at from −100° C. to 60° C., preferably at from −78° C. to 40° C. The reaction time varies depending on the scale of the reaction and the reaction temperature, but is generally between a few minutes and 48 hours.

After completion of the reaction, the compound of Formula 5 is separated from the reaction mixture by one of the conventional separation techniques. If necessary, the compounds are purified by recrystallization, distillation or chromatography or, if desired, can also be used in the next step without prior purification.

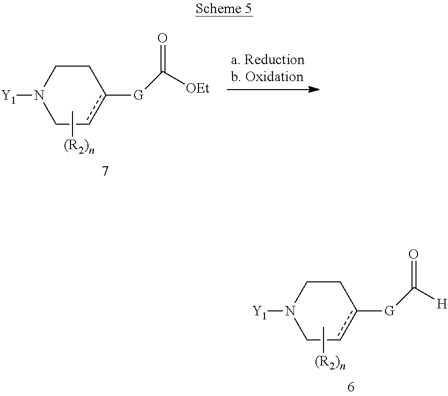

As shown in Scheme 5, synthesis of the compound of Formula 6 involves (step a) simple one-pot reduction reaction of compound of Formula 7 into the corresponding alcohol using NaBH₄-MeOH system. The aromatic alcohols were obtained by the method explained in the ARKIVOC 2006, 128-133, involving the reduction of aromatic ethyl esters within 15 to 60 minutes after refluxing in THF.

The corresponding alcohol is oxidized into the compound of Formula 6 (step b) using oxidizing agents like MnO₂, Dess-Martin periodinane, 2-iodoxybenzoic acid (IBX) and (2,2,6,6-Tetramethylpiperidin-1-yl)oxyl or (2,2,6,6-tetramethylpiperidin-1-yl)oxidanyl (TEMPO). Preferred solvents for the such reaction is acetonitrile or Dichloromethane. For references, see Dess, D. B.; Martin, J. C. J. Am. Chem. Soc. 1991, 113, 7277, Quesada, E.; Taylor, R. J. K., Tetrahedron Lett. 2005, 46, 6473-6476. Naik, N.; Braslau, R. Tetrahedron 1998, 54,667).

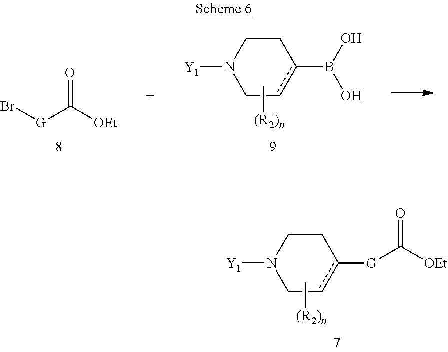

The compound of Formula 7 is prepared by Suzuki reaction involving Pd-catalyzed cross-coupling of an iodide or bromide of compound of Formula 8 with a boronic acid or ester of compound of Formula 9, as shown in Scheme 6. Many catalysts are useful for this type of transformation; a typical catalyst is tetrakis (triphenylphosphine) palladium, or Bis (triphenylphosphine) palladium chloride. Solvents such as tetrahydrofuran, acetonitrile, diethyl ether-dioxane or dioxane:water mixtures are suitable for Suzuki reaction. Suzuki reaction and related coupling procedures offer many alternatives for creation of the C-G bond. For references, see for example C. A. Zificsak and D. J. Hlasta, Tetrahedron 2004, 60, 8991-9016. For a thorough review of palladium chemistry applicable to the synthesis of C-G bonds see J. J. Li and G. W. Gribble, editors, Palladium in Heterocyclic Chemistry: A Guide for the Synthetic Chemist, Elsevier: Oxford, UK, 2000. Many variations of catalyst type, base and reaction conditions are known in the art for this general method.

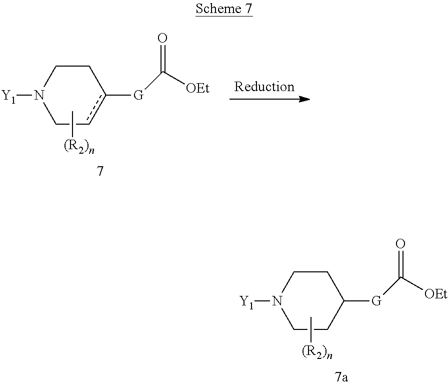

Reduction of the endocyclic double bond in a compound of Formula 7 is carried out using catalytic hydrogenation to give a compound of Formula 7a. Pd/C is the preferred catalyst. For references, see for example Sarah Sulzer-Mosse et al Bioorganic & Medicinal Chemistry 2015 23 2129-2138.

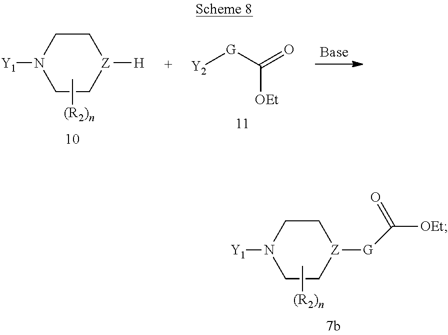

A compound of Formula 7b, wherein Z is a nitrogen atom, can be prepared by displacement of an appropriate leaving group Y₂ on a compound of Formula 11 by a nitrogen-containing heterocycle of Formula 10 in the presence of a base as depicted in Scheme 8. Suitable bases for such reaction include sodium hydride or potassium carbonate. The reaction is carried out in a solvent such as N,N-dimethylformamide or acetonitrile at 0 to 80° C. Suitable leaving groups in the compound of Formula 11 include bromide, iodide, mesylate (OS(O)₂CH₃), triflate (OS(O)₂CF₃) and the like. The compound of Formula 11 can be prepared from the corresponding compound wherein Y₂ is OH, using general methods known in the art.

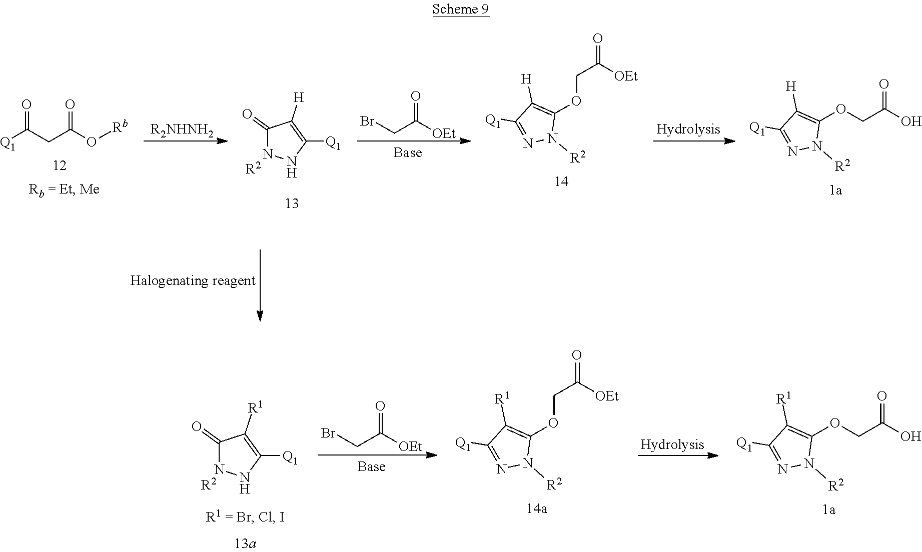

The starting β-ketoesters of Formula 12 and hydrazines of formula R²NHNH₂ are commercially available or can be prepared by methods well-known in the art. β-ketoesters of Formula 12 are reacted with hydrazines of formula R²NHNH₂ to form intermediates of Formula 13. For relevant references, see Katrizky et al., J. Chem. Soc. Perkin Trans. II 1987, 969-975; Muller et al., Monatshefle fuer Chemie 1958, 89, 23-35; WO2006/116713 and US2007/0049574.

A compound of Formula 13a wherein R¹ is halogen can be prepared from the compound of Formula 13 (R¹ is H) by treating with a halogenating reagent as shown in Scheme 9. A variety of halogenating reagents known in the art are suitable for this method including, for example, N-halosuccinimides (e.g., NBS, NCS, NIS), elemental halogen (e.g., Cl₂, Br₂, I₂), phosphorus oxyhalides, phosphorus trihalides, phosphorus pentahalides, thionyl chloride, sulfuryl chloride, bis(pyridine)iodonium(I) tetrafluoroborate, tetramethylammonium iodide. Particularly, useful as halogenating reagents are N-halosuccinimides.

Typically, the reaction is carried out in a suitable solvent such as N, N-dimethylformamide, carbon tetrachloride, acetonitrile, dichloromethane, acetic acid, chloroform, benzene, xylenes, chlorobenzene, tetrahydrofuran, 1, 4 dioxane or the like. Optionally, an organic base such as triethylamine, pyridine, N, N-dimethylaniline, or the like can be added. Catalysts such as N, N dimethylformamide or 2, 2′-azobis (2-methylpropionitrile) (AIBN) may also be used in such reactions. Reaction temperatures range from about room temperature (e.g., 20° C.) to 150° C. For representative procedures, see US2007/0049574; WO2006/071730; Campos et al., Tetrahedron Letters 1997, and Gibert et al., Pharmaceutical Chemistry Journal 2007, 41(3), 154-156.

Compounds of Formula 14 and 14a can be prepared by treating the compounds of Formula 13 and 13a respectively with ethyl bromo acetate preferably with bases as shown in the scheme 9. The reaction is carried out in a suitable solvent such as N, N-dimethylformamide, carbon tetrachloride, acetonitrile, dichloromethane, tetrahydrofuran, acetone 1, 4 dioxane, or the like. Optionally, an organic base such as triethylamine, pyridine, or inorganic bases such as K₂CO₃, Cs₂CO₃, Ag₂CO₃, Na₂CO₃ or the like can be added.

Compounds of Formula 14 and 14a can be further hydrolyzed by treating them with sodium hydroxide or lithium hydroxide to get compound of Formula 1a as shown in the scheme 8. Preferred solvents for the hydrolysis conditions are water, ethanol, or tetrahydrofuran.

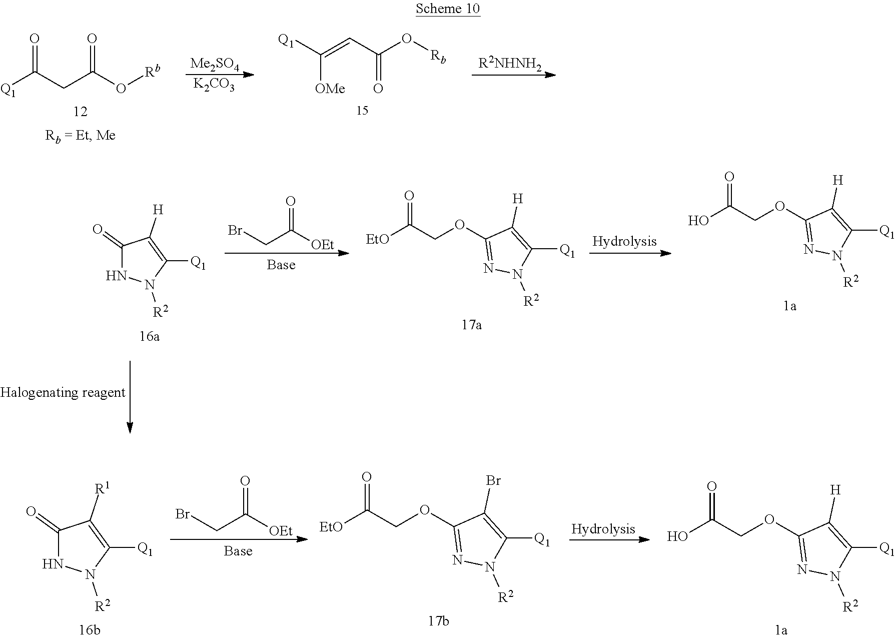

The β-ketoesters of Formula 12 and hydrazines of formula R²NHNH₂ are commercially available or can be prepared methods well-known in the art. A compound Formula 15 can be prepared by reacting the compound of Formula 12 and dimethyl sulfate in the presence of bases like K₂CO₃,Cs₂CO₃,Ag₂CO₃,Na₂CO₃. The compound of Formula 15 is then reacted with hydrazine of Formula R²NHNH₂ in protic solvents like ethanol or methanol to obtain a compound of Formula 16a as explained in Journal of Heterocyclic Chemistry, 1993, 30, 1, 49-54.

A compound of Formula 16b, wherein R¹ is halogen, can be prepared from the compound of Formula 16a (R¹ is H) by treatment with a halogenating reagent as shown in Scheme 10. A variety of halogenating reagents known in the art are suitable for this method including, for example, N-halosuccinimides (e.g., NBS, NCS, NIS), elemental halogen (e.g., Cl₂, Br₂, I₂), phosphorus oxyhalides, phosphorus trihalides, phosphorus pentahalides, thionyl chloride, sulfuryl chloride, bis(pyridine)iodonium(I) tetrafluoroborate, tetramethylammonium iodide. Particularly useful as halogenating reagents are N-halosuccinimides. Typically, the reaction is carried out in a suitable solvent such as N, N-dimethylformamide, carbon tetrachloride, acetonitrile, dichloromethane, acetic acid, chloroform, benzene, xylenes, chlorobenzene, tetrahydrofuran, 1, 4 dioxane or the like. Optionally, an organic base such as triethylamine, pyridine, N, N-dimethylaniline, or the like can be added. Catalyst such as N, N dimethylformamide or 2, 2′-azobis (2-methylpropionitrile) (AIBN) can be used in such reactions. Reaction temperatures range from about room temperature (e.g., 20° C.) to 150° C. For representative procedures, see US2007/0049574; WO2006/071730; Campos et al., Tetrahedron Letters 1997, JS(48), 8397-8400 and Gibert et al., Pharmaceutical Chemistry Journal 2007, 41(3), 154-156.

Compounds of Formula 17a and 17b can be prepared by treating compounds of Formula 16a and 16b, respectively with ethyl bromo acetate preferably in the presence of bases as shown in the scheme 10. Typically, the reaction is carried out in a suitable solvent such as N, N-dimethylformamide, carbon tetrachloride, acetonitrile, dichloromethane, tetrahydrofuran, acetone, 1, 4 dioxane, or the like. Optionally, an organic base such as triethylamine, pyridine, or inorganic bases such as K₂CO₃, Cs₂CO₃, Ag₂CO₃, Na₂CO₃ or the like can be added.

Compounds of Formula 17a and 17b can be further hydrolyzed by treating it with sodium hydroxide or lithium hydroxide to get the compound of Formula 1a as shown in the scheme 10. Preferred solvents for the hydrolysis conditions are water, ethanol, or tetrahydrofuran.

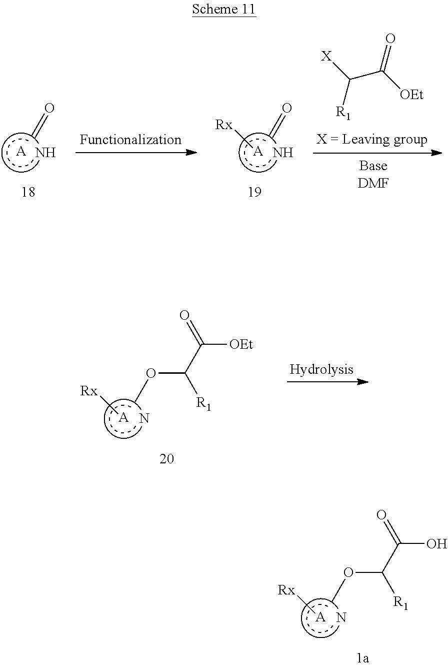

A compound of Formula 1a can be obtained as described in Scheme 11. Suitably substituted compound of Formula 18 can be purchased commercially or can be prepared from the corresponding chloro derivatives using known methods in the literature. Best reagents for these conversions are sulfuric acid, hydrochloric acid, sodium hydroxide. For representative procedures, see WO2007/39563 WO2014/71044, Lavecchia; Berteina-Raboin; Guillaumet, Tetrahedron Letters, 2004, vol. 45, 35, 6633-6636.

Substituted compounds of the formula 18 can be further functionalized using known methods in the literature like chlorination, Bromination, Trifluromethylation to get appropriately substituted heterocyclic ring like Pyridone (Formula 19) References for the said transformations are Zhang, Pei-Zhi et al Tetrahedron, 2016, 72(23), 3250-3255; Canibano; Rodriguez; Santos; Sanz-Tejedor; Carreno; Gonzalez; Garcia-Ruano Synthesis, 2001, 14,2175-2179, WO2004/50637.

A substituted functionalized heterocyclic ring containing a pyridone-like moiety can be alkylated by reaction with an alkyl ester containing a suitable leaving group such as halogen, mesylate or tosylate, in the presence of a base such as Ag₂CO₃ or Cs₂CO₃, in a polar solvent such as DMF or NMP, or non polar solvent such as toluene, xylene with or without heating to get the compound of Formula 20. Typically mixtures of O- and N-alkylated products are obtained, and the two regio-isomeric products can be separated by means of SiO₂ gel or reverse phase chromatography. The addition of lithium salts, for example LiCl, to the reaction mixture can be done to favor N- vs. O-alkylation. The obtained alkyl ester can be further hydrolyzed to the corresponding acids by heating or stirring at room temperature in the presence of lithium hydroxide or sodium hydroxide in solvents like ethanol, water to get the novel compound of Formula 1.

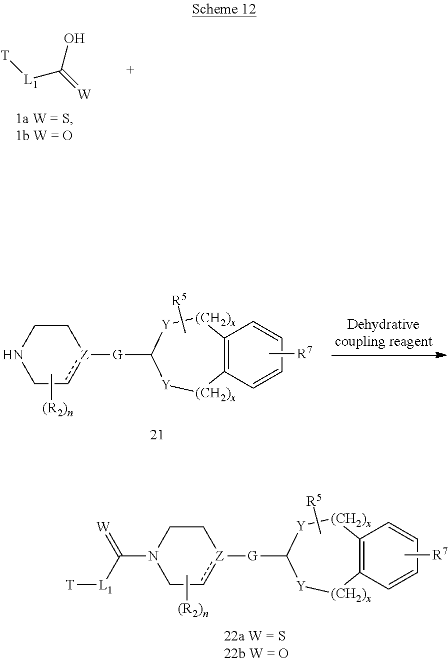

As shown in Scheme 12, a compound of Formula 22a or 22b is prepared by coupling a compound of Formula 1a or 1b respectively, with an amine of Formula 21 (or its acid salt) in the presence of a dehydrative coupling reagent such as dicyclohexylcarbodiimide (DCC), 1-(3-dimethylaminopropyl)-3-ethylcarbodiimide hydrochloride (EDC) O-benzotriazol-1-yl-tetramethyluronium hexafluoro-phosphate (HBTU), or 1-[Bis(dimethylamino)methylene]-1H-1,2,3-triazolo[4,5-b]pyridinium 3-oxid hexafluorophosphate (HATU). Polymer-supported reagents such as polymer-bound cyclohexylcarbodiimide can also be used for these reactions. These reactions are typically carried out at 0-40° C. in a solvent such as dichloromethane, acetonitrile or N,N-dimethylformamide in the presence of a base such as triethylamine or diisopropylethylamine.

Alternatively, the compound of Formula 22b can be obtained by reacting the compound of Formula 22a with a variety of standard thiating reagents such as phosphorus pentasulfide or 2, 4-bis (4-methoxyphenyl)-1, 3-dithia-2, 4-diphosphetane-2, 4-disulfide (Lawesson's reagent).

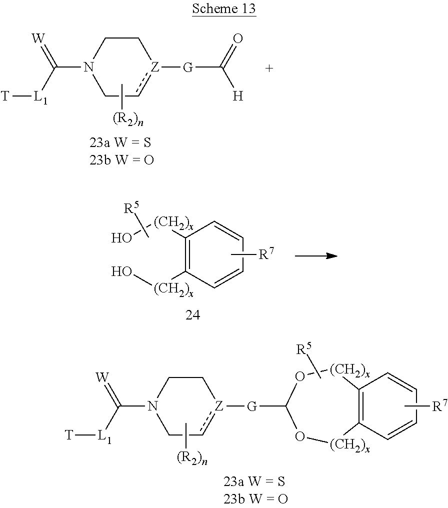

As shown in the scheme 13, the compound of Formula 22 can be prepared by treating a compound of Formula 23 with a compound of Formula 24 in the presence of an acid or a Lewis acid, preferably in the presence of an acid.

Examples of the acid which can be used in this step include inorganic acids such as hydrochloric acid, hydrobromic acid, sulfuric acid and the like; organic acids such as acetic acid, trifluoroacetic acid, p-toluenesulfonic acid, trifluoromethanesulfonic acid and the like.

Examples of the Lewis acid which can be used in this step include zinc chloride, aluminum chloride, tin chloride, boron trichloride, boron trifluoride, trimethylsilyltrifluoromethane sulfonate and the like.

The solvent which can be used in this step may be any solvent which does not inhibit the progress of this reaction and examples thereof include nitriles such as acetonitrile; ethers such as diethyl ether, diisopropyl ether, tetrahydrofuran, dioxane, monoglyme, diglyme, etc.; dichloromethane, dichloroethane, Halogenated hydrocarbons such as chloroform, carbon tetrachloride and tetrachloroethane; aromatic hydrocarbons such as benzene, chlorobenzene, nitrobenzene and toluene; amides such as N,N-dimethylformamide and N, N-dimethylacetamide; imidazolinones such as 1,3-dimethyl-2-imidazolinone, sulfur compounds such as dimethylsulfoxide and the like can be used, and mixed solvents thereof can also be used.

The reaction temperature may be selected from the range of −20° C. to the boiling point of the inert solvent being used, preferably in the range of 0° C. to 150° C.

The reaction time varies depending on the reaction temperature, the reaction substrate, the reaction amount and the like, but is usually from 10 minutes to 48 hours.

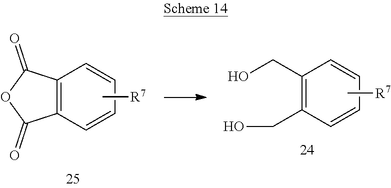

The compound of Formula 24 can be prepared by reducing the compound of Formula 25 with a reducing agent in a solvent as shown in the scheme 14. Reducing agent suitable in this step are lithium aluminum hydride, diisobutylaluminum hydride, borane and the like. Preferred solvent that can be used in this step is tetrahydrofuran, dioxane or like.

The reaction temperature may be selected from the range of from −20° C. to the boiling point of the inert solvent to be used, preferably in the range of 0° C. to 100° C.

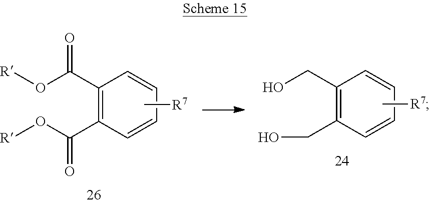

The compound of Formula 24 can also be prepared by reducing a compound of Formula 26 with a reducing agent in a solvent as shown in the scheme 15. Reducing agent suitable in this step, are lithium aluminum hydride, diisobutylaluminum hydride, borane and the like. Preferred solvent that can be used in this step is tetrahydrofuran, dioxane or like.

The reaction temperature may be selected from the range of from −20° C. to the boiling point of the inert solvent to be used, preferably in the range of 0° C. to 100° C.

The present invention shall now be described with non-limiting specific examples.

### Example 1

### Preparation of 1-(4-(4-(5-(2, 6-difluorophenyl)-4, 5-dihydroisoxazol-3-yl) thiazol-2-yl) piperidin-1-yl)-2-((1-methyl-3-(trifluoromethyl)-1H-pyrazol-5-yl) oxy) ethan-1-one (Compound 3)

### Step A: Preparation of ethyl 2-bromo-1,3-thiazole-4-carboxylate

To a solution of ethyl 2-aminothiazole-4-carboxylate (100 g, 581 mmol) and copper (11) bromide (195 g, 871 mmol) in acetonitrile (1 L) at 0° C., tert-butylnitrite (104 mL, 871 mmol) was added dropwise. The resulting reaction mixture was warmed to 25° C. and stirred for 12 h. The reaction mixture was diluted with ethyl acetate (1 L) and water (3 L) and acidified to pH 2 using IN hydrochloric acid. Two layers were separated, the aqueous layer was extracted three times with ethyl acetate (500 mL) and dried over anhydrous sodium sulphate, concentrated and purified by recrystallization with hexane to obtain the title compound (115 g, 84% yield).

¹H-NMR (400 MHz, DMSO-d₆) δ 8.52 (s, 1H), 4.29 (q, J=7.1 Hz, 2H), 1.29 (t, J=7.1 Hz, 3H) MS: m/z=235.90. [M+1].

### Step B: Preparation of ethyl 2-(1-(tert-butoxycarbonyl)-1,2,3,6-tetrahydropyridin-4-yl)thiazole-4-carboxylate

Bis(triphenylphosphine)palladium(II)chloride (9.46 g, 13.5 mmol), tert-butyl 4-(4,4,5,5-tetramethyl-1,3,2-dioxaborolan-2-yl)-3,6-dihydropyridine-1(2H)-carboxylate (100 g, 323 mmol) and a solution of sodium carbonate (86 g, 809 mmol) in water (100 mL) are consecutively added to a solution of ethyl 2-bromothiazole-4-carboxylate (63.6 g, 270 mmol) in dioxane (200 mL). The resulting reaction mixture was heated to 85° C. for 12 h. The reaction mixture was cooled to 25° C. filtered through celite bed and washed with methanol. The filtrate was concentrated, purified by column chromatography using 25% ethyl acetate and hexane as an eluent to give ethyl 2-(1-(tert-butoxycarbonyl)-1, 2, 3, 6-tetrahydropyridin-4-yl) thiazole-4-carboxylate (50 g, 55% yield).

¹H-NMR (400 MHz, DMSO-d₆) δ 8.40 (s, 1H), 6.63 (s, 1H), 4.26 (q, J=7.0 Hz, 2H), 4.01 (s, 2H), 3.49 (t, J=5.7 Hz, 2H), 2.54 (d, J=1.7 Hz, 2H), 1.39 (d, J=6.4 Hz, 9H), 1.24-1.28 (m, 3H). MS: m/z=339 [M+1].

### Step C: Preparation of ethyl 2-(1-(tert-butoxycarbonyl)piperidin-4-yl)thiazole-4-carboxylate

To a solution of ethyl 2-(1-(tert-butoxycarbonyl)-1,2,3,6-tetrahydropyridin-4-yl)thiazole-4-carboxylate (12.8 g, 37.8 mmol) in ethanol (200 mL), a suspension of 10% palladium on charcoal (16.1 g, 15.1 mmol) in ethanol (100 mL) was added. The resulting reaction mixture was maintained under hydrogen pressure of 70 bar at 65° C. for 12 h. The reaction mixture was cooled to 25° C. and filtered. The filtrate was concentrated to afford ethyl 2-(1-(tert-butoxycarbonyl) piperidin-4-yl) thiazole-4-carboxylate (9.3 g, 72% yield).

¹H-NMR (400 MHz, DMSO-d₆) δ 8.41 (s, 1H), 4.28 (q, J=7.1 Hz, 2H), 4.00 (d, J=12.5 Hz, 2H), 3.20-3.27 (m, 1H), 2.87 (s, 2H), 2.00-2.03 (m, 2H), 1.53 (ddd, J=24.7, 12.2, 4.1 Hz, 2H), 1.37-1.43 (m, 9H), 1.28 (t, J=7.1 Hz, 3H). MS: m/z=341.10 [M+1].

### Step D: Preparation of tert-butyl 4-(4-(hydroxymethyl)thiazol-2-yl)piperidine-1-carboxylate

To a stirred solution of ethyl 2-(1-(tert-butoxycarbonyl) piperidin-4-yl) thiazole-4-carboxylate (30 g, 88 mmol) in tetrahydrofuran (500 mL), sodium borohydride (16.6 g, 441 mmol) was added and heated to 60° C. Methanol (40 mL) was added slowly into the reaction mixture, quenched with ammonium chloride solution (200 mL) and extracted twice with dichloromethane (200 mL). The combined dicloromethane layer was dried over anhydrous sodium sulphate and concentrated to obtain 4-(4-(hydroxymethyl) thiazol-2-yl) piperidine-1-carboxylate (21 g, 80% yield).

MS: m/z=299.401 [M+1].

### Step E: Preparation of tert-butyl 4-(4-formylthiazol-2-yl)piperidine-1-carboxylate

To a solution of tert-butyl 4-(4-(hydroxymethyl) thiazol-2-yl) piperidine-1-carboxylate (8.4 g, 28.2 mmol) in dichloromethane (350 ml), Dess-Martin periodinane (23.8 g, 56.3 mmol) was added. The resulting reaction mixture was allowed to stir for 12 h at 25° C. and quenched with aqueous sodium bicarbonate solution. The aqueous layer was extracted twice with dichloromethane (200 mL). The combined dichloromethane layer was dried over anhydrous sodium sulphate, concentrated and purified by column chromatography using 30% of ethyl acetate and hexane as an eluent to obtain tert-butyl 4-(4-formylthiazol-2-yl)piperidine-1-carboxylate (5.3 g, 52% yield).

¹H-NMR (400 MHz, DMSO-d₆) 9.87 (s, 1H), 8.63 (s, 1H), 4.00 (d, J=13.0 Hz, 2H), 3.24-3.29 (m, 1H), 2.89 (s, 2H), 2.04 (dd, J=12.7, 1.8 Hz, 2H), 1.56 (ddd, J=24.6, 12.1, 4.1 Hz, 2H), 1.38-1.43 (m, 9H).

MS: m/z=297.385 [M+1].

### Step F: Preparation of tert-butyl (E/Z)-4-(4-((hydroxyimino)methyl)thiazol-2-yl)piperidine-1-carboxylate

To a stirred solution of hydroxylamine hydrochloride (0.6 g, 8.1 mmol) in ethanol (15 mL), pyridine (1.3 mL 16.2 mmol) was added. After 10 min, tert-butyl 4-(4-formylthiazol-2-yl) piperidine-1-carboxylate (2 g, 6.7 mmol) was added and stirred for 1 h at 25° C. The resulting reaction mixture was concentrated, quenched with aqueous ammonium chloride solution (20 mL) and extracted twice with ethyl acetate (50 mL). The combined ethyl acetate layer was dried over anhydrous sodium sulphate and concentrated to obtain tert-butyl (E/Z)-4-(4-((hydroxyimino) methyl) thiazol-2-yl) piperidine-1-carboxylate (2 g, 95% yield).

¹H-NMR (400 MHz, DMSO-d₆) 12.05-11.09 (1H), 8.49-7.96 (1H), 7.87-7.54 (1H), 3.99 (d, J=14.1 Hz, 2H), 3.25-3.13 (m, 1H), 2.88 (s, 2H), 2.01 (dd, J=12.8, 2.1 Hz, 2H), 1.61-1.45 (m, 2H), 1.39 (s, 9H).

MS: m/z=312.400 [M+1].

### Step G: Preparation of tert-butyl 4-(4-(5-(2,6-difluorophenyl)-4,5-dihydroisoxazol-3-yl)thiazol-2-yl)piperidine-1-carboxylate

To a stirred solution of tert-butyl (E/Z)-4-(4-((hydroxyimino)methyl)thiazol-2-yl)piperidine-1-carboxylate (0.5 g, 1.7 mmol) in dry tetrahydrofuran (30 mL), 2,6-difluorostyrene (0.4 mL, 3.4 mmol) was added at 0° C., followed by 4% aqueous sodium hypochlorite (6.2 mL, 5.1 mmol) and stirred for 1 h. The reaction was quenched with water and the aqueous layer was extracted twice with ethyl acetate (50 mL). The combined ethyl acetate layer was dried over anhydrous sodium sulphate, concentrated and purified by column chromatography using 30% ethyl acetate and hexane as an eluent to obtain tert-butyl 4-(4-(5-(2,6-difluorophenyl)-4,5-dihydroisoxazol-3-yl)thiazol-2-yl)piperidine-1-carboxylate (250 mg, 0.6 mmol, 33% yield).

### Step G1: Alternate Preparation of tert-butyl 4-(4-(5-(2,6-difluorophenyl)-4,5-dihydroisoxazol-3-yl)thiazol-2-yl)piperidine-1-carboxylate

To a stirred solution of tert-butyl (E/Z)-4-(4-((hydroxyimino) methyl) thiazol-2-yl) piperidine-1-carboxylate (4 g, 12.8 mmol) in ethyl acetate (100 mL), N-chlorosuccinimide (3.4 g, 25.7 mmol) was added followed by sodium bicarbonate (7.5 g, 90 mmol). To the resulting reaction mixture, 1, 3-difluoro-2-vinylbenzene (3.6 g, 25.7 mmol) and water (10 mL) were added. The reaction mixture was heated to 65° C. for 3 h, cooled to 15° C. and quenched with water. The aqueous layer was extracted twice with ethyl acetate (50 mL). The combined ethyl acetate layer was dried over anhydrous sodium sulphate concentrated and purified by column chromatography using 30% ethyl acetate and hexane as an eluent to obtain the tert-butyl 4-(4-(5-(2, 6-difluorophenyl)-4, 5-dihydroisoxazol-3-yl) thiazol-2-yl) piperidine-1-carboxylate (3 g, 6.6 mmol, 52% yield).

¹H-NMR (400 MHz, DMSO-d₆) δ 8.00 (s, 1H), 7.46-7.51 (m, 1H), 7.15 (t, J=8.5 Hz, 2H), 5.99 (dd, J=12.1, 8.6 Hz, 1H), 4.00 (d, J=12.5 Hz, 2H), 3.88 (dd, J=17.3, 12.1 Hz, 1H), 3.47-3.55 (m, 1H), 3.24-3.28 (m, 1H), 3.05-2.74 (m, 2H), 2.03 (dd, J=12.8, 2.1 Hz, 2H), 1.55 (dd, J=12.2, 4.1 Hz, 2H), 1.40 (s, 9H) MS: m/z=450.20 [M+1].

### Step H: Preparation of 5-(2,6-difluorophenyl)-3-(2-(piperidin-4-yl)thiazol-4-yl)-4,5-dihydroisoxazole

To a solution of tert-butyl 4-(4-(5-(2, 6-difluorophenyl)-4,5-dihydroisoxazol-3-yl) thiazol-2-yl) piperidine-1-carboxylate (0.2 g, 0.5 mmol) in dichloromethane (20 mL), trifluoroacetic acid (1.5 mL, 18.8 mmol) was added. The resulting reaction mixture was stirred at 25° C. for 1 h, concentrated and quenched with aqueous sodium bicarbonate solution. The aqueous layer was extracted twice with ethyl acetate (25 mL) and the combined ethyl acetate layer was dried over anhydrous sodium sulphate and concentrated to obtain 160 mg of 5-(2,6-difluorophenyl)-3-(2-(piperidin-4-yl)thiazol-4-yl)-4,5-dihydroisoxazole.

### Step I: Preparation of 1-(4-(4-(5-(2,6-difluorophenyl)-4,5-dihydroisoxazol-3-yl)thiazol-2-yl)piperidin-1-yl)-2-((1-methyl-3-(trifluoromethyl)-1H-pyrazol-5-yl)oxy)ethan-1-one

To a solution of amine 5-(2,6-difluorophenyl)-3-(2-(piperidin-4-yl)thiazol-4-yl)-4,5-dihydroisoxazole (0.1 g, 0.3 mmol) and 2-((1-methyl-3-(trifluoromethyl)-1H-pyrazol-5-yl)oxy)acetic acid (0.1 g, 0.5 mmol), in N, N-dimethylformamide (5 mL), HATU (0.2 g, 0.5 mmol) were added followed by N, N-diisopropylethylamine (0.3 mL, 1.7 mmol) and stirred for 16 h at 25° C. The reaction mixture was diluted with water (20 mL) and extracted twice with ethyl acetate (20 mL). The combined ethyl acetate layer was dried over anhydrous sodium sulphate, concentrated and purified by column chromatography using 70% ethyl acetate and hexane as an eluent to obtain 1-(4-(4-(5-(2,6-difluorophenyl)-4,5-dihydroisoxazol-3-yl)thiazol-2-yl)piperidin-1-yl)-2-((1-methyl-3-(trifluoromethyl)-1H-pyrazol-5-yl)oxy)ethan-1-one (0.13 g, 0.24 mmol, 68% yield).

¹H-NMR (400 MHz, DMSO-d₆) δ 8.01 (s, 1H), 7.45-7.50 (m, 1H), 7.12-7.18 (m, 2H), 6.19 (s, 1H), 5.99 (dd, J=12.1, 8.6 Hz, 1H), 5.06 (dd, J=26.7, 15.0 Hz, 2H), 4.36 (d, J=13.1 Hz, 1H), 3.88 (dd, J=17.2, 12.2 Hz, 1H), 3.76 (d, J=13.9 Hz, 1H), 3.67 (s, 3H), 3.51 (q, J=8.7 Hz, 1H), 3.33-3.39 (m, 1H), 3.18 (t, J=11.8 Hz, 1H), 2.77-2.82 (m, 1H), 2.08 (d, J=12.5 Hz, 2H), 1.77 (d, J=12.2 Hz, 1H), 1.54 (d, J=11.2 Hz, 1H) MS: m/z=556.25 [M+1].

### Example 2

### Preparation of 1-(4-(4-(5-(2, 6-difluorophenyl)-4, 5-dihydroisoxazol-3-yl) thiazol-2-yl) piperidin-1-yl)-2-((1-methyl-5-(trifluoromethyl)-1H-pyrazol-3-yl) oxy) ethan-1-one (Compound 20)

To a solution of amine 5-(2,6-difluorophenyl)-3-(2-(piperidin-4-yl)thiazol-4-yl)-4,5-dihydroisoxazole (0.16 g, 0.4 mmol) and the 2-((1-methyl-5-(trifluoromethyl)-1H-pyrazol-3-yl)oxy)acetic acid (0.1 g, 0.6 mmol) in N, N-dimethylformamide (5 mL), HATU (0.2 g, 0.5 mmol) was added followed by N, N-diisopropylethylamine (0.3 mL, 1.7 mmol) and stirred for 16 h at 25° C. The reaction mixture was diluted with water (20 mL) and extracted twice with ethyl acetate (20 mL). The combined ethyl acetate layer was dried over anhydrous sodium sulphate and concentrated and purified by column chromatography using 80% ethyl acetate and hexane as an eluent to obtain 1-(4-(4-(5-(2,6-difluorophenyl)-4,5-dihydroisoxazol-3-yl)thiazol-2-yl)piperidin-1-yl)-2-((1-methyl-5-(trifluoromethyl)-1H-pyrazol-3-yl)oxy)ethan-1-one (0.07 g, 0.13 mmol, 29% yield).

¹H-NMR (400 MHz, DMSO-d₆) δ 8.01 (s, 1H), 7.42-7.50 (m, 1H), 7.09-7.15 (m, 2H), 6.38 (d, J=15.1 Hz, 1H), 5.96 (dd, J=12.2, 8.6 Hz, 1H), 4.71-5.05 (m, 2H), 4.34 (d, J=12.4 Hz, 1H), 3.77-3.91 (m, 2H), 3.74 (s, 3H), 3.49 (q, J=8.6 Hz, 1H), 3.31-3.38 (m, 1H), 3.14-3.21 (m, 1H), 2.73-2.79 (m, 1H), 1.99-2.06 (m, 2H), 1.70-1.79 (m, 1H), 1.50-1.57 (m, 1H) MS: m/z=556.45 [M+1].

### Example 3

### Preparation of 1-(4-(5-(2, 6-difluorophenyl)-4, 5-dihydroisoxazol-3-yl) thiazol-2-yl) piperidin-1-yl)-2-((3-(trifluoromethyl)pyridin-2-yl) oxy) ethan-1-one (Compound 29)

To a solution of amine 5-(2,6-difluorophenyl)-3-(2-(piperidin-4-yl)thiazol-4-yl)-4,5-dihydroisoxazole (0.1 g, 0.4 mmol), 2-((3-(trifluoromethyl)pyridin-2-yl)oxy)acetic acid (0.1 g, 0.4 mmol) in N, N-dimethylformamide (5 mL), HATU (0.2 g, 0.6 mmol), N, N-diisopropylethylamine (0.4 ml, 2.1 mmol) were added and stirred for 16 h at 25° C. The reaction mixture was diluted with water (20 mL) and extracted twice with ethyl acetate (20 mL). The combined ethyl acetate layer was dried over anhydrous sodium sulphate, concentrated and purified by column chromatography using 80% ethyl acetate and hexane as an eluent to obtain 1-(4-(4-(5-(2, 6-difluorophenyl)-4, 5-dihydroisoxazol-3-yl) thiazol-2-yl) piperidin-1-yl)-2-((3-(trifluoromethyl) pyridin-2-yl) oxy) ethan-1-one (0.2 g, 0.3 mmol, 72% yield).

¹H-NMR (400 MHz, DMSO-d₆) δ 8.39 (dd, J=4.9, 1.1 Hz, 1H), 8.10 (dd, J=7.6, 1.1 Hz, 1H), 8.02 (s, 1H), 7.45-7.53 (m, 1H), 7.12-7.17 (m, 3H), 6.00 (dd, J=12.0, 8.6 Hz, 1H), 5.26 (s, 2H), 4.32 (d, J=13.1 Hz, 1H), 3.89 (dd, J=17.1, 12.1 Hz, 2H), 3.53 (q, J=8.7 Hz, 1H), 3.39 (qd, J=7.7, 4.1 Hz, 1H), 3.20-3.24 (m, 1H), 2.76-2.82 (m, 1H), 2.04-2.11 (m, 2H), 1.77-1.86 (m, 1H), 1.55 (dd, J=20.9, 10.8 Hz, 1H) MS: m/z=553.50 [M+1].

### Example 4

### Preparation of 3-(2-(1-(2-((3-(trifluoromethyl)pyridin-2-yl)oxy)acetyl)piperidin-4-yl)thiazol-4-yl)-1,5-dihydrobenzo[e][1,3]dioxepin-6-yl methanesulfonate (Compound 91)

To a solution 3-(2-(piperidin-4-yl)thiazol-4-yl)-1,5-dihydrobenzo[e][1,3]dioxepin-6-yl methanesulfonate (0.2 g, 0.5 mmol) in N, N-diisopropylethylamine (0.6 mL, 3.4 mmol), HATU (0.28 g, 0.7 mmol) and 2-((3-(trifluoromethyl)pyridin-2-yl)oxy)acetic acid (0.1 g, 0.5 mmol) in N, N-dimethylformamide (4 mL) were added and stirred for 2 h at 25° C. The resulting reaction mixture was quenched with water (400 mL) and extracted twice with ethylacetate (500 mL). The combined ethyl acetate layer was dried over anhydrous sodium sulphate, filtered, concentrated and purified by column chromatography using 80% ethyl acetate and hexane as an eluent to obtain 3-(2-(1-(2-((3-(trifluoromethyl)pyridin-2-yl)oxy)acetyl)piperidin-4-yl)thiazol-4-yl)-1,5-dihydrobenzo[e][1,3]dioxepin-6-yl methanesulfonate (40 mg, 0.1 mmol, 14% yield).

¹H-NMR (400 MHz, DMSO-d₆) δ 8.40 (d, J=4.9 Hz, 1H), 8.12 (d, J=7.6 Hz, 1H), 7.64 (s, 1H), 7.38 (t, J=7.7 Hz, 1H), 7.28-7.32 (m, 2H), 7.17 (dd, J=7.3, 5.4 Hz, 1H), 6.08 (s, 1H), 5.28 (s, 2H), 5.17 (d, J=15.2 Hz, 1H), 4.95-5.08 (m, 3H), 4.33 (d, J=14.2 Hz, 1H), 3.91 (d, J=12.7 Hz, 1H), 3.50 (s, 3H), 3.36-3.40 (m, 1H), 3.23-3.24 (m, 1H), 2.79 (s, 1H), 2.09 (d, J=14.9 Hz, 2H), 1.88-1.73 (m, 1H), 1.62-1.45 (m, 1H).

### Example 5

### Preparation of 2-((1-methyl-3-(trifluoromethyl)-1H-pyrazol-5-yl) oxy) acetic acid (IN-5)

### Step A: Preparation of 2-methyl-5-(trifluoromethyl)-1,2-dihydro-3H-pyrazol-3-one

To a solution of ethyl 4, 4, 4-trifluoro-3-oxobutanoate (20 g, 109 mmol) in ethanol (150 mL), methyl hydrazine (10 g, 217 mmol) was added. The resulting reaction mixture was stirred at 85° C. for 16 h. Ethanol was evaporated, water (200 mL) was added and filtered to obtain 2-methyl-5-(trifluoromethyl)-1, 2-dihydro-3H-pyrazol-3-one (11 g, 66 mmol, 61% yield).

¹H-NMR (400 MHz, DMSO-d₆) δ 11.67 (s, 1H), 5.70 (s, 1H), 3.57 (s, 3H) MS: m/z=167.10 (M+1)

### Step B: Preparation of ethyl 2-((1-methyl-3-(trifluoromethyl)-1H-pyrazol-5-yl)oxy)acetate

To a solution of 2-methyl-5-(trifluoromethyl)-1,2-dihydro-3H-pyrazol-3-one (1 g, 6 mmol) in acetone (20 mL), sodium carbonate (3.2 g, 30 mmol) was added at 25° C. and stirred for 10 min to obtain a reaction mass. Ethyl 2-bromoacetate (1.2 g, 7.2 mmol) was added to the reaction mass and stirred at 60° C. for 16 h. The reaction mixture was cooled to 25° C., filtered and concentrated to obtain ethyl 2-((1-methyl-3-(trifluoromethyl)-1H-pyrazol-5-yl) oxy) acetate (1 g, 4 mmol, 66% yield).

¹H-NMR (400 MHz, DMSO-d₆) δ 6.25 (s, 1H), 4.93 (s, 2H), 4.11-4.19 (m, 2H), 3.68 (s, 3H), 1.18-1.22 (m, 3H) MS: m/z=253.10 (M+1)

### Step C: Preparation of 2-((1-methyl-3-(trifluoromethyl)-1H-pyrazol-5-yl)oxy)acetic acid

To a solution of ethyl 2-((1-methyl-3-(trifluoromethyl)-1H-pyrazol-5-yl) oxy) acetate (1 g, 4 mmol) in tetrahydrofuran (8 mL), ethanol (2 mL) and water (1 mL), lithium hydroxide monohydrate (0.8 g, 19.8 mmol) was added. The resulting reaction mixture was stirred at 25° C. for 3 h. The reaction mixture was concentrated, diluted with water and acidified with 6N hydrochloric acid (pH 4) to obtain solids. The solids were filtered and dried to obtain 2-((1-methyl-3-(trifluoromethyl)-1H-pyrazol-5-yl) oxy) acetic acid (0.6 g, 2.7 mmol, 68% yield).

¹H-NMR (400 MHz, DMSO-d₆) δ 13.71-12.39 (1H), 6.21 (s, 1H), 4.81 (s, 2H), 3.63-3.71 (m, 3H) MS: m/z=222.95 (M−1)

### Example 6

### Preparation of 2-((1-methyl-5-(trifluoromethyl)-1H-pyrazol-3-yl) oxy) acetic acid (IN-3)

### Step A: Preparation of 1-methyl-5-(trifluoromethyl)-1,2-dihydro-3H-pyrazol-3-one

To a solution of ethyl (E/Z)-4, 4, 4-trifluoro-3-methoxybut-2-enoate (3 g, 15.1 mmol) in ethanol (30 mL), methyl hydrazine (2.1 g, 45.4 mmol) was added. The resulting reaction mixture was stirred at 25° C. for 16 h. The reaction mixture was concentrated and diluted with water (25 mL), extracted twice with ethyl acetate (25 mL). The combined ethyl acetate layer was dried over anhydrous sodium sulphate and concentrated to obtain 1-methyl-5-(trifluoromethyl)-1,2-dihydro-3H-pyrazol-3-one (2.1 g, 12.6 mmol, 84% yield).

¹H-NMR (400 MHz, DMSO-d₆) δ 9.92 (s, 1H), 6.01 (s, 1H), 3.69 (dd, J=12.8, 1.0 Hz, 3H) MS: m/z=164.95 (M−1)

### Step B: Preparation of ethyl 2-((1-methyl-5-(trifluoromethyl)-1H-pyrazol-3-yl)oxy)acetate

To a solution of 1-methyl-5-(trifluoromethyl)-1,2-dihydro-3H-pyrazol-3-one (2 g, 12 mmol) in acetone (40 mL), sodium carbonate (6.4 g, 60.2 mmol) was added at 25° C. to obtain a reaction mass. To the reaction mass ethyl 2-bromoacetate (2.4 g, 14.5 mmol) was added. The resulting reaction mixture was stirred at 60° C. for 16 h. The reaction mixture was filtered. The filtrate was concentrated to obtain ethyl 2-((1-methyl-5-(trifluoromethyl)-1H-pyrazol-3-yl)oxy)acetate (2.2 g, 8.7 mmol, 72% yield).

¹H-NMR (400 MHz, DMSO-d₆) δ 6.34-6.44 (s, 1H), 6, 4.71-4.76 (s, 2H), 4.01-4.18 (m, 2H), 3.66-3.81 (s, 3H), 1.10-1.26 (m, 3H) MS: m/z=253.00 (M+1)

### Step C: Preparation of 2-((1-methyl-5-(trifluoromethyl)-1H-pyrazol-3-yl)oxy)acetic acid

To a solution of ethyl 2-((1-methyl-5-(trifluoromethyl)-1H-pyrazol-3-yl)oxy)acetate (1.2 g, 4.8 mmol) in a mixture of tetrahydrofuran (16 mL), ethanol (4 mL) and water (2 mL), lithium hydroxide monohydrate (1 g, 23.8 mmol) was added. The resulting reaction mixture was stirred at 25° C. for 3 h, concentrated, diluted with water, acidified with 5N hydrochloric acid (pH 4) and extracted twice with ethyl acetate (30 mL). The ethyl acetate layer was dried over anhydrous sodium sulphate and concentrated to obtain 2-((1-methyl-5-(trifluoromethyl)-1H-pyrazol-3-yl) oxy) acetic acid (0.8 g, 3.6 mmol, 75% yield).

¹H-NMR (400 MHz, DMSO-d₆) δ 12.88 (s, 1H), 6.38 (s, 1H), 4.65-4.71 (m, 2H), 3.70-3.80 (m, 3H) MS: m/z=222.90 (M−1)

### Example 7

### General Scheme for Synthesis Pyridonoxy Acid

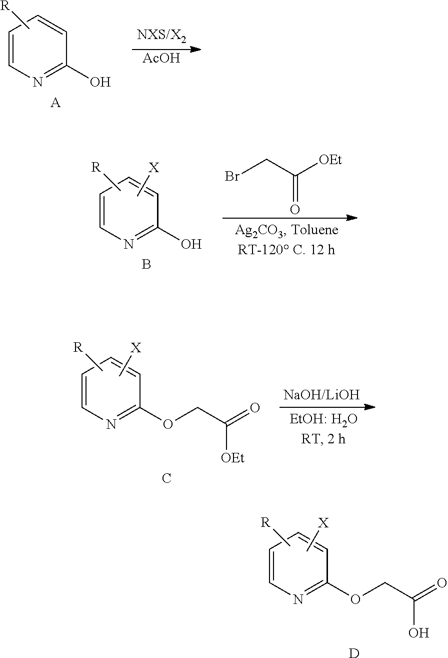

### Step 1: Preparation of B (Halogenation)

To a stirred solution of substituted 2-pyridone (1 equiv.) in acetic acid (10 mL), N-halo succinamide (1.5 equiv.) was added and heated to 120° C. for 16 h. The resulting reaction mixture was filtered, concentrated, diluted with saturated aqueous NaHCO₃ and extracted twice with ethyl acetate. Then ethyl acetate layer was washed with brine (50 mL), dried over anhydrous sodium sulphate, filtered, concentrated and purified by column chromatography to give B.

### Step 1A: Alternative Preparation of B (Halogenation)

To a solution of substituted 2-pyridone (1 equiv.) in dichloromethane (15 mL), bromine (1.2 equiv.) was added slowly. After the completion of addition, the reaction mixture was stirred at 25° C. for 18 h. The reaction mixture was cooled to 0° C. and quenched with sodium bicarbonate solution (5 mL). The reaction mixture was then extracted twice with ethyl acetate (25 mL). The combined ethyl acetate layer was dried over anhydrous sodium sulphate, concentrated and purified by column chromatography using 50% ethyl acetate and hexane as an eluent to obtain compound B.

### Step 1B: Alternative Preparation of B (Trifluoromethylation)

To a solution of substituted 2-pyridone (9.2 mmol, 1 equiv.) and sodium trifluoromethanesulfinate (27.6 mmol, 3 equiv.) in acetic acid (10 mL), manganese triacetate hydrate (27.6 mmol, 3 equiv.) was added in lots. The resulting reaction mixture was stirred at 25° C. for 12 h, water (20 mL) was added to the reaction mixture, extracted twice with ethyl acetate (25 mL). The combined ethyl acetate layer was dried over anhydrous sodium sulphate, concentrated and purified by column chromatography using 70% ethyl acetate and hexane as an eluent to obtain compound B.

### Step 2: Preparation of C (Alkylation)

To a solution of substituted 2-pyridone (1.2 mmol, 1 equiv.) and silver carbonate (3.7 mmol, 3 equiv.) in toluene (5 mL), ethyl 2-bromoacetate (3.6 mmol, 3 equiv.) was added. The resulting reaction mixture was stirred at 100° C. for 16 h, cooled to 25° C., diluted with water and extracted twice with ethyl acetate (15 mL). The combined ethyl acetate layers were concentrated and purified by column chromatography to obtain compound C.

### Step 3: Preparation of D (Hydrolysis)

To a solution of compound C (9.2 mmol, 1 euqiv.) in ethanol and water, sodium hydroxide (18.5 mmol, 2 equiv.) was added and the resulting reaction mixture was stirred at 25° C. for 2 h. The reaction mixture was concentrated and diluted with water, acidified with 6N hydrochloric acid to pH 4. The precipitated solid product was filtered, washed with water followed by n-hexane and dried to obtain corresponding pyridonoxy acid D.

The pyridonoxy/pyrazoloxy acids as seen in Table no 1 were prepared analogously by the procedure described in the Examples 5, 6 and 7.

TABLE 1

| | | | LCMS |
|---|---|---|---|
| S. No | Structure | ¹H NMR | (M + 1)/(M − 1) |
| IN-1 | 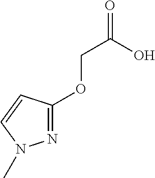 | — | 157 |
| IN-2 | 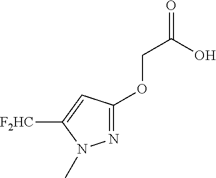 | ¹H-NMR (400 MHz, DMSO-d₆) δ 12.89 (s, 1H), 7.19 (t, J = 53.5 Hz, 1H), 6.04 (s, 1H), 4.62 (d, J = 15.3 Hz, 2H), 3.71 (s, 3H) | 207.05 |
| IN-3 | 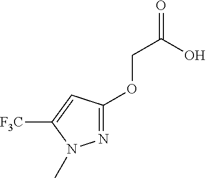 | ¹H-NMR (400 MHz, DMSO-d₆) δ 12.88 (s, 1H), 6.38 (s, 1H), 4.65-4.71 (m, 2H), 3.70-3.80 (m, 3H) | 222.90 |
| IN-4 | 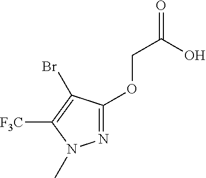 [SMILES: Cn1nc(OCC(=O)O)c(Br)c1C(F)(F)F] | ¹H-NMR (400 MHz, DMSO-d₆) δ 13.08 (s, 1H), 4.76 (s, 2H), 3.83 (d, J = 1.2 Hz, 3H) | 302.80 |
| IN-5 | 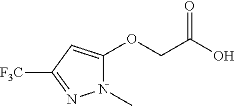 | ¹H-NMR (400 MHz, DMSO-d₆) δ 13.71-12.39 (1H), 6.21 (s, 1H), 4.81 (s, 2H), 3.63-3.71 (m, 3H) | 222.95 |
| IN-6 | 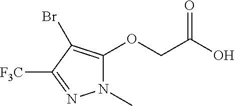 [SMILES: Cn1nc(C(F)(F)F)c(Br)c1OCC(=O)O] | ¹H-NMR (400 MHz, DMSO-d₆) δ 13.01 (s, 1H), 4.98 (s, 2H) 3.77 (d J = 8.9 Hz 3H) | 303 |
| IN-7 | 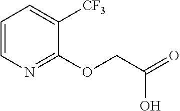 [SMILES: O=C(O)COc1ncccc1C(F)(F)F] | ¹H-NMR (400 MHz, DMSO-d₆) δ 13.03 (s, 1H), 8.39 (d, J = 4.6 Hz, 1H), 8.12 (d, J = 7.5 Hz, 1H), 7.19 (dd, J = 7.2, 5.3 Hz, 1H), 4.95 (s, 2H) | 221.90 |
| IN-8 | 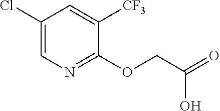 [SMILES: O=C(O)COc1ncc(Cl)cc1C(F)(F)F] | ¹H-NMR (400 MHz, DMSO-d₆) δ 13.07 (s, 1H), 8.50 (d, J = 2.3 Hz, 1H), 8.29 (d, J = 2.4 Hz, 1H), 4.97 (s, 2H) | 253.95 |
| IN-9 | 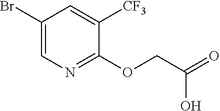 [SMILES: O=C(O)COc1ncc(Br)cc1C(F)(F)F] | ¹H-NMR (400 MHz, DMSO-d₆) δ 13.07 (s, 1H), 8.57 (d, J = 2.3 Hz, 1H), 8.36 (d, J = 2.3 Hz, 1H), 4.95 (d, J = 12.8 Hz, 2H) | 297.85 |
| IN-10 | 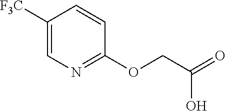 [SMILES: O=C(O)COc1ccc(C(F)(F)F)cn1] | ¹H-NMR (400 MHz, DMSO-d₆) δ 12.97 (s, 1H), 8.56 (d, J = 0.9 Hz, 1H), 8.10 (dd, J = 8.7, 2.4 Hz, 1H), 7.11 (d, J = 8.7 Hz, 1H), 4.91 (s, 2H) | 222.05 |
| IN-11 | 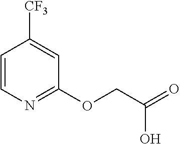 [SMILES: O=C(O)COc1cc(C(F)(F)F)ccn1] | ¹H-NMR (400 MHz, DMSO-d₆) δ 12.94 (s, 1H), 8.40 (d, J = 5.4 Hz, 1H), 7.36 (dd, J = 5.4, 1.0 Hz, 1H), 7.30-7.31 (m, 1H), 4.90 (s, 2H) | 221.13 |
| IN-12 | 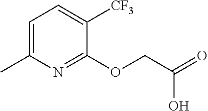 [SMILES: Cc1ccc(C(F)(F)F)c(OCC(=O)O)n1] | ¹H-NMR (400 MHz, DMSO-d₆) δ 13.26 (s, 1H), 7.85 (d, J = 7.6 Hz, 1H), 6.32 (d, J = 7.6 Hz, 1H), 4.77 (s, 2H), 2.36 (s, 3H) | 236.10 |
| IN-13 | 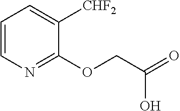 [SMILES: O=C(O)COc1ncccc1C(F)F] | | 204 |

The compounds listed in Table no 2 are prepared in a manner analogous to the procedure described in the examples 1, 2, 3 and 4.

TABLE 2

| | | | LCMS |
|---|---|---|---|
| Com- | | | (M + |
| pound | Compound Name | 1H NMR | H) |
| 1 | 1-(4-(4-(5-(2,6- | ¹H-NMR (400 MHz, | 488.15 |
| | difluorophenyl)- | DMSO-d₆) δ 7.98 (s, 1H), | |
| | 4,5-dihydroisoxazol- | 7.40-7.49 (m, 2H), 7.09- | |
| | 3-yl)thiazol- | 7.15 (m, 2H), 5.96 (dd, J = | |
| | 2-yl)piperidin-1-yl)- | 12.1, 8.6 Hz, 1H), 5.59 | |
| | 2-((1-methyl- | (d, J = 2.3 Hz, 1H), 4.77 | |
| | 1H-pyrazol-3- | (dd, J = 24.4, 13.8 Hz, 2H), | |
| | yl)oxy)ethan-1-one | 4.35 (d, J = 13.1 Hz, | |
| | | 1H) 3.86 (dd, J = 17.1, 12.1 | |
| | | Hz, 2H), 3.62 (d, J = | |
| | | 14.8, Hz, 3H), 3.49 (q, J = | |
| | | 8.6 Hz, 1H), 3.35 (td, J = | |
| | | 7.7, 3.8 Hz, 1H), 3.13-3.19 | |
| | | (m, 1H), 2.72-2.78 | |
| | | (m, 1H), 2.02-2.06 (m, 2H), | |
| | | 1.68-1.78 (m, 1H), | |
| | | 1.36-1.55 (m, 1H) | |
| 2 | 1-(4-(4-(5-(2,6- | ¹H-NMR (400 MHz, | 590 |
| | dichlorophenyl)- | DMSO-d₆) δ 8.02 (s, 1H), | |
| | 4,5-dihydroisoxazol- | 7.54 (d, J = 7.8 Hz, 2H), | |
| | 3-yl)thiazol- | 7.43 (dd, J = 8.8, 7.3 Hz, | |
| | 2-yl)piperidin-1- | 1H), 6.29-6.34 (m, 1H), | |
| | yl)-2-1H-methyl- | 6.20 (s, 1H), 5.06 (dd, J = | |
| | 3-(trifluoromethyl)- | 26.1, 14.9 Hz, 2H), 4.36 | |
| | 1H-pyrazol-5- | (d, J = 12.8 Hz, 1H), | |
| | yl)oxy)ethan-1-one | 3.75-3.89 (m, 2H), 3.67 | |
| | | (s, 3H), 3.55 (dd, J = | |
| | | 17.3, 11.0 Hz, 1H), 3.35- | |
| | | 3.39 (m, 1H), 3.18 (t, J = | |
| | | 11.8 Hz, 1H), 2.80 (t, J = | |
| | | 11.9 Hz, 1H), 2.09 (d, J = | |
| | | 9.9 Hz, 2H), 1.77 (d, J = | |
| | | 11.6 Hz, 1H), 1.53- | |
| | | 1.56 (m, 1H) | |
| 4 | 3-chloro-2-(3-(2- | ¹H-NMR (400 MHz, | 648.15 |
| | (1-(2-((1-methyl- | DMSO-d₆) δ 8.04 (s, 1H), | |
| | 3-(trifluoromethyl)- | 7.45-7.57 (m, 3H), 6.12- | |
| | 1H-pyrazol-5- | 6.20 (m, 2H), 5.06 (dd, J = | |
| | yl)oxy)acetyl)piperidin- | 26.6, 15.1 Hz, 2H), 4.36 | |
| | 4-yl)thiazol-4-yl)-4,5- | (d, J = 12.4 Hz, 1H), | |
| | dihydroisoxazol- | 3.75-3.84 (m, 2H), 3.67 | |
| | 5-yl)phenyl | (s, 3H), 3.50-3.57 (m, | |
| | methanesulfonate | 4H), 3.34-3.39 (m, 1H), | |
| | | 3.18 (t, J = 11.7 Hz, 1H), | |
| | | 2.77-2.83 (m, 1H), 2.08 | |
| | | (d, J = 11.8 Hz, 2H), 1.77 | |
| | | (d, J = 11.5 Hz, 1H), 1.54 | |
| | | (d, J = 12.1 Hz, 1H) | |
| 5 | 3-chloro-2-(3-(2- | ¹H-NMR (400 MHz, | 580.2 |
| | (1-(2-((1-methyl- | DMSO-d₆) δ 8.03 (s, 1H), | |
| | 1H-pyrazol-3- | 7.44-7.57 (m, 4H), 6.12- | |
| | yl)oxy)acetyl)piperidin- | 6.18 (m, 1H), 5.62 (d, J = | |
| | 4-yl)thiazol-4-yl)-4,5- | 2.3 Hz, 1H), 4.80 (dd, J = | |
| | dihydroisoxazol-5- | 24.1, 13.5 Hz, 2H), 4.38 | |
| | yl)phenyl | (d, J = 12.5 Hz, 1H), | |
| | methanesulfonate | 3.77-3.88 | |
| | | (m, 2H), 3.62 (d, J = | |
| | | 10.2 Hz, 3H), 3.50-3.57 | |
| | | (m, 4H), 3.33-3.39 (m, | |
| | | 1H), 3.19 (t, J = 12.3 Hz, | |
| | | 1H), 2.78 (t, J = 11.7 | |
| | | Hz, 1H), 2.07 (d, J = 11.9 | |
| | | Hz, 2H), 1.75 (d, J = | |
| | | 11.0 Hz, 1H), 1.53 (d, | |
| | | J = 11.2 Hz, 1H) | |
| 6 | 1-(4-(4-(5-(2,6- | ¹H-NMR (400 MHz, | 570.1 |
| | dichlorophenyl)- | DMSO-d₆) δ 8.01 (s, 1H), | |
| | 4,5-dihydroisoxazol- | 7.54 (d, J = 7.9 Hz, 2H), | |
| | 3-yl)thiazol- | 7.43 (dd, J = 8.8, 7.4 Hz, | |
| | 2-yl)piperidin- | 1H), 7.19 (t, J = 53.6 Hz, | |
| | 1-yl)-2-((5- | 1H), 6.28-6.34 (m, 1H), | |
| | (difluoromethyl)- | 6.05 (s, 1H), 4.82-4.91 | |
| | 1-methyl-1H- | (m, 2H), 4.37 (d, J = 12.4 | |
| | pyrazol-3- | Hz, 1H), 3.86 (dd, J = 17.1, | |
| | yl)oxy)ethan-1-one | 12.4 Hz, 2H), 3.70 (s, | |
| | | 3H), 3.55 (dd, J = 17.3, | |
| | | 11.0 Hz, 1H), 3.34-3.39 | |
| | | (m, 1H), 3.19 (t, J = 12.3 | |
| | | Hz, 1H), 2.78 (t, J = | |
| | | 11.6 Hz, 1H), 2.07 (d, J = | |
| | | 10.9 Hz, 2H), 1.76 (d, J = | |
| | | 10.1 Hz, 1H), 1.54 (d, | |
| | | J = 9.6 Hz, 1H) | |
| 7 | 2-((5-(difluoromethyl)- | ¹H-NMR (400 MHz, | 606.45 |
| | 1-methyl- | DMSO-d₆) δ 8.02 (s, 1H), | |
| | 1H-pyrazol-3- | 7.76 (s, 2H), 7.19 (t, J = | |
| | yl)oxy)-1-(4-(4-(5- | 53.5 Hz, 1H), 6.28 (dd, J = | |
| | (2,4,6-trichlorophenyl)- | 12.2, 11.0 Hz, 1H), 6.05 | |
| | 4,5-dihydroisoxazol- | (s, 1H), 4.81-4.91 (m, | |
| | 3-yl)thiazol-2- | 2H), 4.37 (d, J = 13.0 Hz, | |
| | yl)piperidin-1- | 1H), 3.86 (dd, J = 17.3, | |
| | yl)ethan-1-one | 12.4 Hz, 2H), 3.70 (s, 3H), | |
| | | 3.51-3.58 (m, 1H), | |
| | | 3.37 (qd, J = 7.7, 3.8 Hz, | |
| | | 1H), 3.19 (t, J = 12.2 | |
| | | Hz, 1H), 2.78 (t, J = 11.8 | |
| | | Hz, 1H), 2.07 (d, J = | |
| | | 11.0 Hz, 2H), 1.76 (dd, | |
| | | J = 21.9, 11.3 Hz, 1H), | |
| | | 1.54 (dd, J = 20.7, 11.4 | |
| | | Hz, 1H) | |
| 8 | 3-chloro-2-(3- | ¹H-NMR (400 MHz, | 630.05 |
| | (2-(1-(2-((5- | DMSO-d₆) δ 8.03 (s, 1H), | |
| | (difluoromethyl)- | 7.45-7.57 (m, 3H), 7.18 | |
| | 1-methyl-1H- | (t, J = 53.5 Hz, 1H), 6.14 | |
| | pyrazol-3- | (dd, J = 12.2, 11.1 Hz, 1H), | |
| | yl)oxy)acetyl)piperidin- | 6.04 (s, 1H), 4.86 (dd, | |
| | 4-yl)thiazol-4-yl)- | J = 25.9 14.3 Hz, 2H) | |
| | 4,5-dihydroisoxazol- | 4.36 (d J = 12.7 Hz, 1H), | |
| | 5-yl)phenyl | 3.77-3.86 (m, 2H), 3.70 | |
| | methanesulfonate | (s, 3H), 3.50-3.57 (m, | |
| | | 4H), 3.33-3.39 (m, 1H), | |
| | | 3.15-3.22 (m, 1H), 2.78 | |
| | | (t, J = 11.7 Hz, 1H), 2.07 | |
| | | (d, J = 11.3 Hz, 2H), | |
| | | 1.76 (d, J = 11.9 Hz, 1H), | |
| | | 1.53 (d, J = 11.0 Hz, | |
| | | 1H) | |
| 9 | 2((5-(difluoromethyl)- | ¹H-NMR (400 MHz, | 538.05 |
| | 1-methyl- | DMSO-d₆) δ 8.01 (s, 1H), | |
| | 1H-pyrazol-3- | 7.44-7.52 (m, 1H), 7.05- | |
| | yl)oxy)-1-(4-(4-(5-(2,6- | 7.32 (m, 3H), 5.96-6.04 | |
| | difluorophenyl)-4,5- | (m, 2H), 4.86 (dd, J = 26.3, | |
| | dihydroisoxazol- | 14.2 Hz, 2H), 4.37 (d, | |
| | 3-yl)thiazol-2- | J = 12.1 Hz, 1H), 3.83-3.92 | |
| | yl)piperidin-1- | (m, 2H), 3.70 (s, 3H), | |
| | yl)ethan-1-one | 3.52 (q, J = 8.6 Hz, 1H), | |
| | | 3.36 (qd, J = 7.7, 3.8 Hz, | |
| | | 1H), 3.16-3.22 (m, 1H), | |
| | | 2.75-2.81 (m, 1H), 2.07 | |
| | | (d, J = 11.8 Hz, 2H), 1.76 | |
| | | (d, J = 11.2 Hz, 1H), | |
| | | 1.54 (d, J = 9.9 Hz, 1H) | |
| 10 | 2-((1-methyl-3- | ¹H-NMR (400 MHz, | 624.25 |
| | (trifluoromethyl)- | DMSO-d₆) δ 8.02 (s, 1H), | |
| | 1H-pyrazol-5- | 7.76 (s, 2H), 6.28 (dd, | |
| | yl)oxy)-1-(4-(4-(5- | J = 12.2, 11.0 Hz, 1H), | |
| | (2,4,6- | 6.19 (s, 1H), 5.06 (dd, | |
| | trichlorophenyl)-4,5- | J = 26.1, 14.9 Hz, 2H), | |
| | dihydroisoxazol- | 4.36 (d, J = 12.8 Hz, 1H), | |
| | 3-yl)thiazol-2- | 3.86 (dd, J = 17.3, 12.3 | |
| | yl)piperidin-1- | Hz, 1H), 3.75-3.78 (m, | |
| | yl)ethan-1-one | 1H), 3.67 (s, 3H), 3.54 | |
| | | (dd, J = 17.3, 10.9 Hz, 1H), | |
| | | 3.33-3.39 (m, 1H), | |
| | | 3.15-3.21 (m, 1H), 2.77-2.83 | |
| | | (m, 1H), 2.08 (d, J = | |
| | | 12.2 Hz, 2H), 1.77 (d, J = | |
| | | 11.6 Hz, 1H), 1.55 (d, J = | |
| | | 11.3 Hz, 1H) | |
| 11 | 2-((4-bromo-1- | ¹H-NMR (400 MHz, | 667.75 |
| | methyl-5- | DMSO-d₆) δ 8.01 (s, 1H), | |
| | (trifluoromethyl)- | 7.53-7.55 (m, 2H), 7.43 | |
| | 1H-pyrazol-3- | (dd, J = 8.8, 7.3 Hz, 1H), | |
| | yl)oxy)-1-(4- | 6.31 (dd, J = 12.2, 11.0 | |
| | (4-(5-(2,6- | Hz, 1H), 5.06 (d, J = 14.8 | |
| | dichlorophenyl)-4,5- | Hz, 1H), 4.96-5.00 (m, | |
| | dihydroisoxazol- | 1H), 4.36 (d, J = 12.2 Hz, | |
| | 3-yl)thiazol-2- | 1H), 3.82-3.89 (m, 5H), | |
| | yl)piperidin-1- | 3.55 (dd, J = 17.3, 11.0 | |
| | yl)ethan-1-one | Hz, 1H), 3.36-3.43 (m, 1H), | |
| | | 3.17-3.25 (m, 1H), | |
| | | 2.77-2.83 (m, 1H), 2.07 | |
| | | (s, 2H), 1.75-1.83 (m, | |
| | | 1H), 1.50-1.59 (m, 1H) | |
| 12 | 2-(3-(2-(1-(2-((4- | ¹H-NMR (400 MHz, | 727.95 |
| | bromo-1-methyl- | DMSO-d₆) δ 8.03 (s, 1H), | |
| | 5-(trifluoromethyl)- | 7.51-7.61 (m, 2H), 7.47 | |
| | 1H-pyrazol-3- | (dd, J = 7.7, 1.8 Hz, 1H), | |
| | yl)oxy)acetyl)piperidin- | 6.12-6.18 (m, 1H), 5.02 | |
| | 4-yl)thiazol-4-yl)-4,5- | (dd, J = 31.9, 14.7 Hz, | |
| | dihydroisoxazol- | 2H), 4.36 (d, J = 12.5 Hz, | |
| | 5-yl)-3- | 1H), 3.77-3.84 (m, 6H), | |
| | chlorophenyl | 3.50-3.57 (m, 4H), 3.34- | |
| | methanesulfonate | 3.41 (m, 2H), 3.20 (t, J = | |
| | | 12.8 Hz, 1H), 2.77-2.83 | |
| | | (m, 1H), 2.07 (s, 2H), | |
| | | 1.78-1.83 (m, 1H), | |
| | | 1.50-1.58 (m, 1H) | |
| 13 | 2-((4-bromo- | ¹H-NMR (400 MHz, | 701.85 |
| | 1-methyl-5- | DMSO-d₆) δ 8.01 (s, 1H), | |
| | (trifluoromethyl)- | 7.76 (s, 2H), 6.28 (dd, | |
| | 1H-pyrazol-3- | J = 12.2, 11.0 Hz, 1H), | |
| | yl)oxy)-1-(4- | 5.06 (d, J = 14.7 Hz, 1H), | |
| | (4-(5-(2,4,6- | 4.96-5.00 (m, 1H), 4.35 | |
| | trichlorophenyl)-4,5- | (d, J = 12.7 Hz, 1H), | |
| | dihydroisoxazol-3- | 3.82-3.90 (m, 5H), 3.54 (dd, | |
| | yl)thiazol-2- | J = 17.3, 10.9 Hz, 1H), | |
| | yl)piperidin- | 3.36-3.41 (m, 1H), 3.20 (t, | |
| | 1-yl)ethan-1-one | J = 12.3 Hz, 1H), 2.80 | |
| | | (t, J = 11.9 Hz, 1H), 2.07 | |
| | | (s, 2H), 1.75-1.83 (m, 1H), | |
| | | 1.50-1.60 (m, 1H) | |
| 14 | 2-((4-bromo- | ¹H-NMR (400 MHz, | 635.95 |
| | 1-methyl-5- | DMSO-d₆) δ 8.01 (s, 1H), | |
| | (trifluoromethyl)- | 7.45-7.52 (m, 1H), 7.12- | |
| | 1H-pyrazol-3- | 7.18 (m, 2H), 5.99 (dd, J = | |
| | yl)oxy)-1-(4- | 12.1, 8.6 Hz, 1H), 5.02 | |
| | (4-(5-(2,6- | (dd, J = 31.6, 14.5 Hz, | |
| | difluorophenyl)-4,5- | 2H), 4.36 (d, J = 13.3 Hz, | |
| | dihydroisoxazol- | 1H), 3.82-3.92 (m, 5H), | |
| | 3-yl)thiazol-2- | 3.52 (q, J = 8.6 Hz, 1H), | |
| | yl)piperidin-1- | 3.34-3.42 (m, 1H), 3.18- | |
| | yl)ethan-1-one | 3.24 (m, 1H), 2.80 (t, | |
| | | J = 11.7 Hz, 1H), 2.07 (s, | |
| | | 2H), 1.78-1.83 (m, 1H), | |
| | | 1.51-1.59 (m, 1H) | |
| 15 | 1-(4-(4-(5-(2,6- | ¹H-NMR (400 MHz, | 590 |
| | dichlorophenyl)- | DMSO-d₆) δ 8.01 (s, 1H), | |
| | 4,5-dihydroisoxazol- | 7.53-7.55 (m, 2H), 7.42 | |
| | 3-yl)thiazol- | (dd, J = 8.8, 7.3 Hz, 1H), | |
| | 2-yl)piperidin-1- | 6.38 (s, 1H), 6.31 (dd, | |
| | yl)-2-((1-methyl- | J = 12.3, 11.1 Hz, 1H), | |
| | 5-(trifluoromethyl)- | 4.90 (dd, J = 27.1, 14.5 Hz, | |
| | 1H-pyrazol-3- | 2H), 4.37 (d, J = 13.1 | |
| | yl)oxy)ethan-1-one | Hz, 1H), 3.80-3.89 (m, | |
| | | 2H), 3.76 (d, J = 0.6 Hz, | |
| | | 3H), 3.55 (dd, J = 17.3, | |
| | | 11.0 Hz, 1H), 3.33-3.39 | |
| | | (m, 1H), 3.19 (t, J = 12.4 | |
| | | Hz, 1H), 2.76-2.81 (m, | |
| | | 1H), 2.07 (d, J = 9.9 Hz, | |
| | | 2H), 1.77 (d, J = 10.9 | |
| | | Hz, 1H), 1.54 (d, J = 9.5 | |
| | | Hz, 1H) | |
| 16 | 2-((4-bromo-1- | ¹H-NMR (400 MHz, | 667.95 |
| | methyl-3- | DMSO-d₆) δ 8.01 (d, J = | |
| | (trifluoromethyl)- | 2.1 Hz, 1H), 7.53-7.55 (m, | |
| | 1H-pyrazol-5- | 2H), 7.43 (dd, J = 8.7, | |
| | yl)oxy)-1-(4- | 7.3 Hz, 1H), 6.31 (dd, | |
| | (4-(5-(2,6- | J = 12.3, 11.1 Hz, 1H), 5.27 | |
| | dichlorophenyl)- | (dd, J = 22.3, 15.1 Hz, 2H), | |
| | 4,5-dihydroisoxazol- | 4.36 (d, J = 13.1 Hz, | |
| | 3-yl)thiazol-2- | 1H), 3.78-3.89 (m, 4H), | |
| | yl)piperidin- | 3.74 (d, J = 15.6 Hz, 1H), | |
| | 1-yl)ethan-1-one | 3.55 (dd, J = 17.2, 10.9 Hz, | |
| | | 1H), 3.34-3.40 (m, | |
| | | 1H), 3.15-3.21 (m, 1H), | |
| | | 2.81 (t, J = 11.5 Hz, 1H), | |
| | | 2.08 (d, J = 12.7 Hz, 2H), | |
| | | 1.75 (d, J = 11.9 Hz, | |
| | | 1H), 1.56 (d, J = 11.8 Hz, | |
| | | 1H) | |
| 17 | 2-(3-(2-(1-(2-((4- | ¹H-NMR (400 MHz, | 727.95 |
| | bromo-1-methyl- | DMSO-d₆) δ 8.03 (s, 1H), | |
| | 3-(trifluoromethyl)- | 7.56 (dd, J = 8.0, 1.9 Hz, | |
| | 1H-pyrazol-5- | 1H), 7.52 (t, J = 7.9 Hz, | |
| | yl)oxy)acetyl)piperidin- | 1H), 7.46 (dd, J = 7.7, | |
| | 4-yl)thiazol-4-yl)-4,5- | 1.9 Hz, 1H), 6.12-6.18 (m, | |
| | dihydroisoxazol- | 1H), 5.00-5.31 (m, 2H), | |
| | 5-yl)-3-chlorophenyl | 4.36 (d, J = 13.1 Hz, 1H), | |
| | methanesulfonate | 3.77-3.84 (m, 4H), 3.73 | |
| | | (d, J = 13.4 Hz, 1H), | |
| | | 3.48-3.57 (m, 4H), 3.37 | |
| | | (td, J = 7.6, 3.8 Hz, 1H), | |
| | | 3.18 (t, J = 11.7 Hz, 1H), | |
| | | 2.78-2.83 (m, 1H), 2.08 | |
| | | (d, J = 12.7 Hz, 2H), 1.76 | |
| | | (t, J = 12.5 Hz, 1H), | |
| | | 1.57 (t, J = 11.8 Hz, 1H) | |
| 18 | 2-((4-bromo- | ¹H-NMR (400 MHz, | 635.95 |
| | 1-methyl-3- | DMSO-d₆) δ 8.01 (d, J = | |
| | (trifluoromethyl)- | 2.9 Hz, 1H), 7.45-7.52 | |
| | 1H-pyrazol-5- | (m, 1H), 7.12-7.17 (m, | |
| | yl)oxy)-1-(4- | 2H), 5.99 (dd, J = 12.1, | |
| | (4-(5-(2,6- | 8.6 Hz, 1H), 5.00-5.32 | |
| | difluorophenyl)-4,5- | (m, 2H), 4.36 (d, J = 13.4 | |
| | dihydroisoxazol- | Hz, 1H), 3.78-3.92 (m, | |
| | 3-yl)thiazol-2- | 4H), 3.74 (d, J = 15.6 Hz, | |
| | yl)piperidin-1- | 1H), 3.52 (q, J = 8.7 | |
| | yl)ethan-1-one | Hz, 1H), 3.34-3.40 (m, 1H), | |
| | | 3.15-3.21 (m, 1H), | |
| | | 2.78-2.84 (m, 1H), 2.06- | |
| | | 2.09 (m, 2H), 1.70-1.80 | |
| | | (m, 1H), 1.58 (t, J = 11.5 | |
| | | Hz, 1H) | |
| 19 | 3-chloro-2-(3-(2- | ¹H-NMR (400 MHz, | 648.05 |
| | (1-(2-((1-methyl- | DMSO-d₆) δ 8.03 (s, 1H), | |
| | 5-(trifluoromethyl)- | 7.56 (dd, J = 7.9, 1.8 Hz, | |
| | 1H-pyrazol-3- | 1H), 7.53 (t, J = 7.9 Hz, | |
| | yl)oxy)acetyl)pipendin- | 1H), 7.47 (dd, J = 7.8, 1.8 | |
| | 4-yl)thiazol-4-yl)-4,5- | Hz, 1H), 6.39 (s, 1H), | |
| | dihydroisoxazol- | 6.15 (dd, J = 12.2, 10.9 Hz, | |
| | 5-yl)phenyl | 1H), 4.91 (dd, J = | |
| | methanesulfonate | 27.8, 14.5 Hz, 2H), 4.37 | |
| | | (d, J = 12.4 Hz, 1H), 3.76- | |
| | | 3.85 (m, 5H), 3.50-3.57 | |
| | | (m, 4H), 3.34-3.40 | |
| | | (m, 1H), 3.19 (t, J = 12.2 | |
| | | Hz, 1H), 2.79 (t, J = | |
| | | 11.8 Hz, 1H), 2.07 (d, J = | |
| | | 10.2 Hz, 2H), 1.77 (d, J = | |
| | | 10.5 Hz, 1H), 1.52-1.56 | |
| | | (m, 1H) | |
| 21 | 2((1-methyl-5- | ¹H-NMR (400 MHz, | 624.2 |
| | (trifluoromethyl)- | DMSO-d₆) δ 8.02 (s, 1H), | |
| | 1H-pyrazol-3- | 7.78 (d, J = 15.0 Hz, 2H), | |
| | yl)oxy)-1-(4-(4-(5- | 6.39 (s, 1H), 6.28 (dd, J = | |
| | (2,4,6- | 12.3, 10.9 Hz, 1H), 4.91 | |
| | trichlorophenyl)-4,5- | (dd, J = 26.9, 14.7 Hz, | |
| | dihydroisoxazol- | 2H), 4.37 (d, J = 12.4 Hz, | |
| | 3-yl)thiazol-2- | 1H), 3.80-3.90 (m, 2H), | |
| | yl)piperidin-1- | 3.76 (d, J = 0.6 Hz, 3H), | |
| | yl)ethan-1-one | 3.54 (dd, J = 17.3, 10.9 | |
| | | Hz, 1H), 3.37 (qd, J = 7.6, | |
| | | 3.8 Hz, 1H), 3.16-3.22 | |
| | | (m, 1H), 2.76-2.82 (m, 1H), | |
| | | 2.07 (d, J = 11.3 Hz, | |
| | | 2H), 1.77 (d, J = 10.9 Hz, | |
| | | 1H), 1.54 (d, J = 9.6 | |
| | | Hz, 1H) | |
| 22 | 2-((4-bromo- | ¹H-NMR (400 MHz, | 702.1 |
| | 1-methyl-3- | DMSO-d₆) δ 8.01 (s, 1H), | |
| | (trifluoromethyl)- | 7.75 (s, 2H), 6.25 (dd, | |
| | 1H-pyrazol-5- | J = 12.3, 10.9 Hz, 1H), | |
| | yl)oxy)-1-(4- | 5.24 (dd, J = 22.1, 14.9 Hz, | |
| | (4-(5-(2,4,6- | 2H), 4.33 (d, J = 13.3 | |
| | trichlorophenyl)-4,5- | Hz, 1H), 3.75-3.86 (m, 4H), | |
| | dihydroisoxazol- | 3.71 (d, J = 13.9 Hz, | |
| | 3-yl)thiazol-2- | 1H), 3.51-3.60 (m, 1H), | |
| | yl)piperidin-1- | 3.33-3.38 (m, 1H), 3.15 | |
| | yl)ethan-1-one | (t, J = 11.8 Hz, 1H), 2.78 | |
| | | (t, J = 11.7 Hz, 1H), | |
| | | 2.03-2.10 (m, 2H), 1.67- | |
| | | 1.76 (m, 1H), 1.49-1.57 | |
| | | (m, 1H) | |
| 23 | 2-(2,4- | ¹H-NMR (400 MHz, | 599.9 |
| | dichlorophenoxy)- | DMSO-d₆) δ 8.02 (d, J = | |
| | 1-(4-(4-(5-(2,6- | 5.7 Hz, 1H), 7.53-7.57 | |
| | dichlorophenyl)-4,5- | (m, 3H), 7.43 (dd, J = 8.7, | |
| | dihydroisoxazol- | 7.3 Hz, 1H), 7.35 (d, J = 8.9 | |
| | 3-yl)thiazol-2- | Hz, 1H), 6.94 (dd, J = | |
| | yl)piperidin-1- | 16.9, 8.9 Hz, 1H), | |
| | yl)propan-1-one | 6.29-6.35 (m, 1H), 5.39 (dd, | |
| | | J = 36.4, 6.6 Hz, 1H), | |
| | | 4.38 (d, J = 13.0 Hz, 1H), | |
| | | 4.08 (q, J = 12.9 Hz, 1H), | |
| | | 3.81-3.90 (m, 1H), | |
| | | 3.51-3.60 (m, 1H), 3.34- | |
| | | 3.39 (m, 1H), 3.20-3.28 | |
| | | (m, 1H), 2.77-2.84 (m, 1H), | |
| | | 2.10 (d, J = 12.5 Hz, | |
| | | 2H), 1.74 (d, J = 11.9 Hz, | |
| | | 1H), 1.45-1.57 (m, 4H) | |
| 24 | 3-chloro-2-(3- | ¹H-NMR (400 MHz, | 659.95 |
| | (2-(1-(2-(2,4- | DMSO-d₆) δ 8.04 (d, J = | |
| | dichlorophenoxy)pro- | 5.5 Hz, 1H), 7.51-7.59 | |
| | panoyl)piperidin- | (m, 3H), 7.47 (dd, J = 7.8, | |
| | 4-yl)thiazol-4-yl)-4,5- | 1.8 Hz, 1H), 7.32-7.36 | |
| | dihydroisoxazol- | (m, 1H), 6.87-7.03 (m, | |
| | 5-yl)phenyl | 1H), 6.12-6.18 (m, 1H), | |
| | methanesulfonate | 5.31-5.45 (m, 1H), 4.38 | |
| | | (d, J = 12.4 Hz, 1H), | |
| | | 4.03-4.14 (m, 1H), 3.76- | |
| | | 3.85 (m, 1H), 3.50-3.58 (m, | |
| | | 4H), 3.34-3.39 (m, 1H), | |
| | | 3.14-3.28 (m, 1H), 2.77- | |
| | | 2.84 (m, 1H), 2.10 (d, J = | |
| | | 11.8 Hz, 2H), 1.74 (d, | |
| | | J = 12.2 Hz, 1H), 1.41-1.57 | |
| | | (m, 4H) | |
| 25 | 2-(2,4- | ¹H-NMR (400 MHz, | 633.75 |
| | dichlorophenoxy)- | DMSO-d₆) δ 8.02 (d, J = | |
| | 1-(4-(4-(5-(2,4,6- | 5.5 Hz, 1H), 7.75 (d, J = | |
| | trichlorophenyl)-4,5- | 10.4 Hz, 2H), 7.57 (d, J = | |
| | dihydroisoxazol- | 2.6 Hz, 1H), 7.35 (d, J = | |
| | 3-yl)thiazol-2- | 8.9 Hz, 1H), 6.94 (dd, | |
| | yl)piperidin-1- | J = 17.1, 9.0 Hz, 1H), | |
| | yl)propan-1-one | 6.25-6.31 (m, 1H), 5.32- | |
| | | 5.45 (m, 1H), 4.38 (d, J = | |
| | | 12.4 Hz, 1H), 4.08 (q, J = | |
| | | 13.0 Hz, 1H), 3.81-3.90 | |
| | | (m, 1H), 3.49-3.58 (m, | |
| | | 1H), 3.33-3.39 (m, 1H), | |
| | | 3.19-3.29 (m, 1H), 2.77- | |
| | | 2.84 (m, 1H), 2.09 (d, J = | |
| | | 12.4 Hz, 2H), 1.74 (d, J = | |
| | | 12.8 Hz, 1H), 1.45-1.57 | |
| | | (m, 4H) | |
| 26 | 2-(2,4- | ¹H-NMR (400 MHz, | 566.05 |
| | dichlorophenoxy)- | DMSO-d₆) δ 8.02 (d, J = | |
| | 1-(4-(4-(5-(2,6- | 5.7 Hz, 1H), 7.56-7.58 | |
| | difluorophenyl)-4,5- | (m, 1H), 7.45-7.53 (m, | |
| | dihydroisoxazol- | 1H), 7.35 (t, J = 4.4 Hz, | |
| | 3-yl)thiazol-2- | 1H), 7.12-7.17 (m, 2H), | |
| | yl)piperidin-1- | 6.95 (dd, J = 16.7, 8.9 Hz, | |
| | yl)propan-1-one | 1H), 5.97-6.02 (m, | |
| | | 1H), 5.32-5.44 (m, 1H), | |
| | | 4.38 (d, J = 13.0 Hz, 1H), | |
| | | 4.03-4.12 (m, 1H), 3.84- | |
| | | 3.93 (m, 1H), 3.47-3.56 | |
| | | (m, 1H), 3.37 (dq, J = 15.0, | |
| | | 3.8 Hz, 1H), 3.20- | |
| | | 3.28 (m, 1H), 2.77-2.84 | |
| | | (m, 1H), 2.10 (d, J = 12.4 | |
| | | Hz, 2H), 1.74 (d, J = 12.1 | |
| | | Hz, 1H), 1.41-1.57 (m, | |
| | | 4H) | |
| 27 | 1-(4-(4-(5-(2,6- | ¹H-NMR (400 MHz, | 585.95 |
| | dichlorophenyl)- | DMSO-d₆) δ 8.38-8.39 (m, | |
| | 4,5-dihydroisoxazol- | 1H), 8.10 (dd, J = 7.5, | |
| | 3-yl)thiazol- | 1.2 Hz, 1H), 8.02 (s, 1H), | |
| | 2-yl)piperidin- | 7.53-7.55 (m, 2H), 7.43 | |
| | 1-yl)-2-((3- | (dd, J = 8.8, 7.3 Hz, 1H), | |
| | (trifluoromethyl)pyridin- | 7.16 (dd, J = 7.3, 5.1 Hz, | |
| | 2-yl)oxy)ethan-1-one | 1H), 6.32 (dd, J = 12.3, | |
| | | 11.1 Hz, 1H), 5.26 (s, 2H), | |
| | | 4.32 (d, J = 12.5 Hz, | |
| | | 1H), 3.83-3.92 (m, 2H), | |
| | | 3.56 (dd, J = 17.1, 11.0 | |
| | | Hz, 1H), 3.36-3.41 (m, | |
| | | 1H), 3.24 (t, J = 12.2 Hz, | |
| | | 1H), 2.79 (t, J = 11.8 Hz, | |
| | | 1H), 2.04-2.11 (m, 2H), | |
| | | 1.81 (d, J = 11.6 Hz, 1H), | |
| | | 1.55 (d, J = 10.4 Hz, | |
| | | 1H) | |
| 28 | 3-chloro-2-(3- | ¹H-NMR (400 MHz, | 645 |
| | (2-(1-(2-((3- | DMSO-d₆) δ 8.39 (d, J = | |
| | (trifluoromethyl)pyridin- | 4.1 Hz, 1H), 8.10 (d, | |
| | 2- | J = 7.5 Hz, 1H), 8.04 (s, | |
| | yl)oxy)acetyl)piperidin- | 1H), 7.51-7.58 (m, 2H), | |
| | 4-yl)thiazol-4-yl)-4,5- | 7.47 (dd, J = 7.8, 1.8 Hz, | |
| | dihydroisoxazol-5- | 1H), 7.16 (dd, J = 7.3, 5.0 | |
| | yl)phenyl | Hz, 1H), 6.13-6.18 (m, | |
| | methanesulfonate | 1H), 5.26 (s, 2H), 4.32 | |
| | | (d, J = 13.0 Hz, 1H), 3.90 | |
| | | (d, J = 13.1 Hz, 1H), 3.82 | |
| | | (dd, J = 17.3, 12.3 Hz, | |
| | | 1H), 3.51-3.58 (m, 4H), | |
| | | 3.36-3.41 (m, 1H), 3.23 | |
| | | (t, J = 12.3 Hz, 1H), 2.78 | |
| | | (t, J = 11.9 Hz, 1H), | |
| | | 2.04-2.10 (m, 2H), 1.81 | |
| | | (d, J = 12.1 Hz, 1H), 1.54 | |
| | | (d, J = 10.5 Hz, 1H) | |
| 30 | 1-(4-(4-(5-(2,4,6- | ¹H-NMR (400 MHz, | 620.9 |
| | trichlorophenyl)- | DMSO-d₆) δ 8.39 (dd, J = | |
| | 4,5-dihydroisoxazol- | 4.8, 1.0 Hz, 1H), 8.10 | |
| | 3-yl)thiazol- | (dd, J = 7.6, 1.1 Hz, 1H), | |
| | 2-yl)piperidin- | 8.02 (s, 1H), 7.76 (s, 2H), | |
| | 1-yl)-2-((3- | 7.16 (dd, J = 7.2, 5.2 | |
| | (trifluoromethyl)pyridin- | Hz, 1H), 6.28 (dd, J = 12.3, | |
| | 2-yl)oxy)ethan-1-one | 11.1 Hz, 1H), 5.26 (s, | |
| | | 2H), 4.32 (d, J = 12.8 Hz, | |
| | | 1H), 3.83-3.92 (m, 2H), | |
| | | 3.55 (dd, J = 17.3, 11.0 | |
| | | Hz, 1H), 3.35-3.40 (m, | |
| | | 1H), 3.22 (d, J = 13.6 Hz, | |
| | | 1H), 2.79 (t, J = 11.9 | |
| | | Hz, 1H), 2.04-2.10 (m, | |
| | | 2H), 1.82 (t, J = 11.7 Hz, | |
| | | 1H), 1.53-1.58 (m, 1H) | |
| 31 | 2-((5-chloro-3- | ¹H-NMR (400 MHz, | 619 |
| | (trifluoromethyl)pyridin- | DMSO-d₆) δ 8.47 (d, J = | |
| | 2-yl)oxy)- | 2.7 Hz, 1H), 8.29 (d, | |
| | 1-(4-(4-(5-(2,6- | J = 2.7 Hz, 1H), 8.04 (s, | |
| | dichlorophenyl)- | 1H), 7.55-7.57 (m, 2H), | |
| | 4,5-dihydroisoxazol- | 7.42 (dd, J = 8.8, 7.3 Hz, | |
| | 3-yl)thiazol- | 1H), 6.32 (dd, J = 12.3, | |
| | 2-yl)piperidin-1- | 11.1 Hz, 1H), 5.30 (s, | |
| | yl)ethan-1-one | 2H), 4.33 (d, J = 13.4 Hz, | |
| | | 1H), 3.85-3.92 (m, 2H), | |
| | | 3.57-3.62 (m, 1H), 3.37- | |
| | | 3.43 (m, 1H), 3.24 (t, J = | |
| | | 11.9 Hz, 1H), 2.80 (t, J = | |
| | | 11.6 Hz, 1H), 2.09 (t, J = | |
| | | 13.8 Hz, 2H), 1.82 (d, | |
| | | J = 11.7 Hz, 1H), 1.56 (d, | |
| | | J = 12.2 Hz, 1H) | |
| 32 | 3-chloro-2-(3-(2- | ¹H-NMR (400 MHz, | 678.95 |
| | (1-(2-((5-chloro-3- | DMSO-d₆) δ 8.48 (t, J = 2.3 | |
| | (trifluoromethyl)pyridin- | Hz, 1H), 8.29 (t, J = 2.2 | |
| | 2- | Hz, 1H), 8.06 (d, J = 1.2 | |
| | yl)oxy)acetyl)piperidin- | Hz, 1H), 7.47-7.61 (m, | |
| | 4-yl)thiazol-4-yl)-4,5- | 3H), 6.14-6.20 (m, 1H), | |
| | dihydroisoxazol- | 5.30 (s, 2H), 4.32 (d, J = | |
| | 5-yl)phenyl | 13.0 Hz, 1H), 3.80-3.90 | |
| | methanesulfonate | (m, 2H), 3.53-3.60 (m, 4H), | |
| | | 3.39-3.41 (m, 1H), | |
| | | 3.24 (m, 1H), 2.78 (m, | |
| | | 1H), 2.07 (d, J = 13.9 Hz, | |
| | | 2H), 1.82 (m, 1H), | |
| | | 1.54 (m, 1H) | |
| 33 | 2-((5-chloro-3- | ¹H-NMR (400 MHz, | 587.4 |
| | (trifluoromethyl)pyridin- | DMSO-d₆) δ 8.49 (d, J = | |
| | 2-yl)oxy)- | 2.0 Hz, 1H), 8.29 (d, | |
| | 1-(4-(4-(5-(2,6- | J = 2.4 Hz, 1H), 8.04 (s, | |
| | difluorophenyl)- | 1H), 7.47-7.54 (m, 1H), | |
| | 4,5-dihydroisoxazol- | 7.14-7.20 (m, 2H), 6.01 | |
| | 3-yl)thiazol- | (dd, J = 12.1, 8.7 Hz, 1H), | |
| | 2-yl)piperidin-1- | 5.30 (s, 2H), 4.31-4.34 | |
| | yl)ethan-1-one | (m, 1H), 3.91 (dd, J = 17.1, | |
| | | 12.2 Hz, 2H), 3.54 (q, | |
| | | J = 8.6 Hz, 1H), 3.37-3.43 | |
| | | (m, 1H), 3.24 (t, J = | |
| | | 12.3 Hz, 1H), 2.68-2.83 | |
| | | (m, 1H), 2.05-2.12 (m, | |
| | | 2H), 1.81-1.87 (m, 1H), | |
| | | 1.54-1.60 (m, 1H) | |
| 34 | 2-((5-chloro-3- | ¹H-NMR (400 MHz, | 654.85 |
| | (trifluoromethyl)pyridin- | DMSO-d₆) δ 8.49 (d, J = | |
| | 2-yl)oxy)- | 2.4 Hz, 1H), 8.29 (d, | |
| | 1-(4-(4-(5-(2,4,6- | J = 2.4 Hz, 1H), 8.04 (s, | |
| | trichlorophenyl)- | 1H), 7.78 (s, 2H), 6.30 | |
| | 4,5-dihydroisoxazol- | (dd, J = 12.2, 11.0 Hz, | |
| | 3-yl)thiazol- | 1H), 5.30 (s, 2H), 4.32 | |
| | 2-yl)piperidin-1- | (d, J = 14.2 Hz, 1H), 3.85- | |
| | yl)ethan-1-one | 3.92 (m, 2H), 3.57 (dd, | |
| | | J = 17.2, 10.9 Hz, 1H), | |
| | | 3.37-3.42 (m, 1H), 3.21- | |
| | | 3.27 (m, 1H), 2.80 (t, J = | |
| | | 12.0 Hz, 1H), 2.05-2.11 | |
| | | (m, 2H), 1.82 (d, J = 12.5 | |
| | | Hz, 1H), 1.57 | |
| | | (t, J = 12.3 Hz, 1H) | |
| 35 | 2-((5-bromo-3- | ¹H-NMR (400 MHz, | 664.85 |
| | (trifluoromethyl)pyridin- | DMSO-d₆) δ 8.55 (d, J = | |
| | 2-yl)oxy)- | 2.4 Hz, 1H), 8.36 (d, | |
| | 1-(4-(4-(5-(2,6- | J = 2.4 Hz, 1H), 8.05 (d, J = | |
| | dichlorophenyl)- | 7.6 Hz, 1H), 7.55-7.57 | |
| | 4,5-dihydroisoxazol- | (m, 2H), 7.45 (dd, J = 8.8, | |
| | 3-yl)thiazol- | 7.3 Hz, 1H), 6.34 (dd, | |
| | 2-yl)piperidin- | J = 12.2, 11.0 Hz, 1H), 5.30 | |
| | 1-yl)ethan-1-one | (s, 2H), 4.32 (d, J = 13.0 | |
| | | Hz, 1H), 3.85-3.92 (m, | |
| | | 2H), 3.58 (dd, J = 17.1, 11.0 | |
| | | Hz, 1H), 3.39 (qd, J = | |
| | | 7.7, 3.8 Hz, 1H), 3.24 (t, | |
| | | J = 12.2 Hz, 1H), 2.80 | |
| | | (t, J = 11.6 Hz, 1H), 2.06- | |
| | | 2.12 (m, 2H), 1.77-1.86 | |
| | | (m, 1H), 1.57 (t, J = 11.7 | |
| | | Hz, 1H) | |
| 36 | 2-(3-(2-(1-(2- | ¹H-NMR (400 MHz, | 723.95 |
| | ((5-bromo-3- | DMSO-d₆) δ 8.55 (d, J = | |
| | (trifluoromethyl)pyridin- | 2.3 Hz, 1H), 8.36 (d, | |
| | 2- | J = 2.2 Hz, 1H), 8.06 (s, J = | |
| | yl)oxy)acetyl)piperidin- | 1.5 Hz, 1H), 7.47-7.59 | |
| | 4-yl)thiazol-4-yl)-4,5- | (m, 3H), 6.14-6.20 (m, | |
| | dihydroisoxazol-5-yl)-3- | 1H), 5.30 (s, 2H), 4.32 | |
| | chlorophenyl | (t, J = 6.2 Hz, 1H), 3.80- | |
| | methanesulfonate | 3.90 (m, 2H), 3.53-3.57 | |
| | | (m, 4H), 3.38-3.41 (m, | |
| | | 1H), 3.24 (t, J = 11.9 Hz, | |
| | | 1H), 2.77-2.83 (m, 1H), | |
| | | 2.05-2.12 (m, 2H), 1.83 | |
| | | (s, 1H), 1.54 (d, J = 3.7 | |
| | | Hz, 1H) | |
| 37 | 2-((5-bromo-3- | ¹H-NMR (400 MHz, | 633.25 |
| | (trifluoromethyl)pyridin- | DMSO-d₆) δ 8.55 (d, J = | |
| | 2-yl)oxy)- | 2.4 Hz, 1H), 8.36 (d, | |
| | 1-(4-(4-(5-(2,6- | J = 2.4 Hz, 1H), 8.05 (d, J = | |
| | difluorophenyl)- | 7.3 Hz, 1H), 7.47-7.57 | |
| | 4,5-dihydroisoxazol- | (m, 1H), 7.14-7.20 (m, | |
| | 3-yl)thiazol- | 2H), 6.01 (dd, J = 12.1, | |
| | 2-yl)piperidin-1- | 8.4 Hz, 1H), 5.30 (s, 2H), | |
| | yl)ethan-1-one | 4.31-4.35 (m, 1H), 3.91 | |
| | | (dd, J = 17.2, 12.1 Hz, | |
| | | 2H), 3.54 (q, J = 8.6 Hz, | |
| | | 1H), 3.37-3.42 (m, 1H), | |
| | | 3.24 (t, J = 11.7 Hz, 1H), | |
| | | 2.77-2.83 (m, 1H), 2.09 | |
| | | (dd, J = 14.1, 11.9 Hz, | |
| | | 2H), 1.82 (d, J = 12.0 Hz, | |
| | | 1H), 1.51-1.57 (m, 1H) | |
| 38 | 2-((5-bromo-3- | ¹H-NMR (400 MHz, | 698.75 |
| | (trifluoromethyl)pyridin- | DMSO-d₆) δ 8.55 (d, J = | |
| | 2-yl)oxy)- | 2.2 Hz, 1H), 8.36 (d, | |
| | 1-(4-(4-(5-(2,4,6- | J = 2.2 Hz, 1H), 8.04 (s, J = | |
| | trichlorophenyl)- | 1.0 Hz, 1H), 7.78 (d, | |
| | 4,5-dihdroisoxazol- | J = 0.7 Hz, 2H), 6.27-6.33 | |
| | 3-yl)thiazol- | (m, 1H), 5.30 (s, 2H), | |
| | 2-yl)piperidm-1- | 4.32 (t, J = 6.6 Hz, 1H), | |
| | yl)ethan-1-one | 3.89 (dd, J = 17.4, 12.5 | |
| | | Hz, 2H), 3.57 (dd, J = | |
| | | 17.2, 10.9 Hz, 1H), 3.38- | |
| | | 3.41 (m, 1H), 3.25 (d, J = | |
| | | 12.2 Hz, 1H), 2.80-2.83 | |
| | | (m, 1H), 2.04-2.12 (m, | |
| | | 2H), 1.83 (m, J = 1.2 | |
| | | Hz, 1H), 1.54 (m, 1H) | |
| 39 | 2-((6-methyl-3- | ¹H-NMR (400 MHz, | 635.05 |
| | (trifluoromethyl)pyridin- | DMSO-d₆) δ 8.03 (s, 1H), | |
| | 2-yl)oxy)- | 7.96 (d, J = 7.6 Hz, 1H), | |
| | 1-(4-(4-(5-(2,4,6- | 7.77 (s, 2H), 7.00 (d, J = | |
| | trichlorophenyl)- | 7.8 Hz, 1H), 6.28 (dd, | |
| | 4,5-dihydroisoxazol- | J = 12.3, 10.9 Hz, 1H), 5.19 | |
| | 3-yl)thiazol- | (dd, J = 40.7, 14.3 Hz, 2H), | |
| | 2-yl)piperidin-1- | 4.34 (d, J = 11.7 Hz, | |
| | yl)ethan-1-one | 1H), 3.94 (d, J = 13.0 Hz, | |
| | | 1H), 3.86 (dd, J = 17.4, | |
| | | 12.2 Hz, 1H), 3.55 (dd, | |
| | | J = 17.2, 10.9 Hz, 1H), | |
| | | 3.36-3.44 (m, 1H), 3.21- | |
| | | 3.28 (m, 1H), 2.76-2.83 | |
| | | (m, 1H), 2.39 (s, 3H), | |
| | | 2.05-2.13 (m, 2H), 1.76- | |
| | | 1.86 (m, 1H), 1.49-1.59 | |
| | | (m, 1H) | |
| 40 | 1-(4-(4-(5-(2,6- | ¹H-NMR (400 MHz, | 567.05 |
| | difluorophenyl)- | DMSO-d₆) δ 8.02 (s, 1H), | |
| | 4,5-dihydroisoxazol- | 7.96 (d, J = 7.6 Hz, 1H), | |
| | 3-yl)thiazol- | 7.55 (d, J = 0.7 Hz, 1H), | |
| | 2-yl)piperidin-1-yl)- | 7.53 (s, 1H), 7.43 (dd, | |
| | 2-((6-methyl-3- | J = 8.7, 7.2 Hz, 1H), 7.00 | |
| | (trifluoromethyl)pyridin- | (d, J = 7.6 Hz, 1H), | |
| | 2-yl)oxy)ethan-1-one | 6.29-6.35 (m, 1H) 5.20 (dd, | |
| | | J = 42.2, 15.0 Hz, 2H), 4.34 | |
| | | (d, J = 13.9 Hz, 1H), | |
| | | 3.94 (d, J = 15.6 Hz, 1H), | |
| | | 3.86 (dd, J = 17.2, 12.3 | |
| | | Hz, 1H), 3.56 (dd, J = 17.1, | |
| | | 11.0 Hz, 1H), 3.36- | |
| | | 3.44 (m, 1H), 3.21-3.28 | |
| | | (m, 1H), 2.80 (t, J = 11.6 | |
| | | Hz, 1H), 2.39 (s, 3H), | |
| | | 2.05-2.14 (m, 2H), 1.76- | |
| | | 1.87 (m, 1H), 1.48-1.60 | |
| | | (m, 1H) | |
| 41 | 1-(4-(4-(5-(2,6- | ¹H-NMR (400 MHz, | 599 |
| | dichlorophenyl)- | DMSO-d₆) δ 8.03 (s, 1H), | |
| | 4,5-dihydroisoxazol- | 7.96 (d, J = 7.8 Hz, 1H), | |
| | 3-yl)thiazol- | 7.46-7.53 (m, 1H), 7.16 | |
| | 2-yl)piperidin-1- | (t, J = 8.6 Hz, 2H), 7.00 | |
| | yl)-2-((6-methyl-3- | (d, J = 7.8 Hz, 1H), 6.00 | |
| | (trifluoromethyl)pyridin- | (dd, J = 12.1, 8.4 Hz, 1H), | |
| | 2-yl)oxy)ethan-1-one | 5.20 (dd, J = 44.7, 14.7 | |
| | | Hz, 2H), 4.35 (d, J = 10.5 | |
| | | Hz, 1H), 4.04-3.80 | |
| | | (2H), 3.53 (dd, J = 17.7, | |
| | | 8.4 Hz, 1H), 3.38-3.43 | |
| | | (m, 1H), 3.22-3.28 (m, 1H), | |
| | | 2.76-2.83 (m, 1H), | |
| | | 2.39 (s, 3H), 2.05-2.14 | |
| | | (m, 2H), 1.76-1.87 (m, | |
| | | 1H), 1.47-1.61 (m, 1H | |
| 42 | 3-chloro-2-(3-(2- | ¹H-NMR (400 MHz, | 659.15 |
| | (1-(2-((6-methyl-3- | DMSO-d₆) δ 8.06 (s, 1H), | |
| | (trifluoromethyl)pyridin- | 7.97 (d, J = 7.8 Hz, 1H), | |
| | 2- | 7.58 (dd, J = 7.9, 1.8 Hz, | |
| | yl)oxy)acetyl)piperidin- | 1H), 7.54 (t, J = 7.9 Hz, | |
| | 4-yl)thiazol-4-yl)-4,5- | 1H), 7.48 (dd, J = 7.8, 1.7 | |
| | dihydroisoxazol- | Hz, 1H), 7.01 (d, J = 7.8 Hz, | |
| | 5-yl)phenyl | 1H), 6.17 (dd, J = | |
| | methanesulfonate | 12.2, 11.0 Hz, 1H), 5.21 | |
| | | (dd, J = 46.0, 14.4 Hz, | |
| | | 2H), 4.36 (d, J = 12.7 Hz, | |
| | | 1H), 3.95 (d, J = 13.2 | |
| | | Hz, 1H), 3.83 (dd, J = 17.2, | |
| | | 12.3 Hz, 1H), 3.56 | |
| | | (dd, J = 17.2, 10.9 Hz, 1H), | |
| | | 3.54 (s, 3H), 3.42 (tt, | |
| | | J = 11.5, 3.9 Hz, 1H), 3.26 | |
| | | (t, J = 12.5 Hz, 1H), | |
| | | 2.81 (t, J = 12.2 Hz, 1H), | |
| | | 2.40 (s, 3H), 2.10 (t, J = | |
| | | 17.0 Hz, 2H), 1.79-1.88 (m, | |
| | | 1H), 1.50-1.59 (m, | |
| | | 1H) | |
| 43 | 1-(4-(4-(5-(2,6- | ¹H-NMR (400 MHz, | 552.5 |
| | difluorophenyl)- | DMSO-d₆) δ 8.38 (d, J = | |
| | 4,5-dihydroisoxazol- | 5.1 Hz, 1H), 8.02 (s, 1H), | |
| | 3-yl)thiazol- | 7.45-7.53 (m, 1H), 7.30 | |
| | 2-yl)piperidin- | (d, J = 5.4 Hz, 1H), 7.27 | |
| | 1-yl)-2-((4- | (s, 1H), 7.12-7.18 (m, | |
| | (trifluoromethyl)pyridin- | 2H), 6.00 (dd, J = 12.1, | |
| | 2-yl)oxy)ethan-1-one | 8.7 Hz, 1H), 5.19 (s, 2H), | |
| | | 4.33 (d, J = 13.0 Hz, 1H), | |
| | | 3.89 (dd, J = 17.2, 12.1 | |
| | | Hz, 2H), 3.53 (dd, J = 17.2, | |
| | | 8.6 Hz, 1H), 3.38 (tt, | |
| | | J = 11.4, 3.7 Hz, 1H), | |
| | | 3.21-3.27 (m, 1H), 2.79 (t, | |
| | | J = 11.6 Hz, 1H), 2.08 (t, | |
| | | J = 15.8 Hz, 2H), 1.81 | |
| | | (dd, J = 24.1, 12.3 Hz, | |
| | | 1H), 1.49-1.59 (m, 1H) | |
| 44 | 1-(4-(4-(5-(2,4,6- | ¹H-NMR (400 MHz, | 619.8 |
| | trichlorophenyl)- | DMSO-d₆) δ 8.38 (d, J = | |
| | 4,5-dihydroisoxazol- | 5.4 Hz, 1H), 8.02 (s, 1H), | |
| | 3-yl)thiazol- | 7.76 (s, 2H), 7.30 (dd, J = | |
| | 2-yl)piperidin-1- | 5.4, 1.5 Hz, 1H), 7.26-7.28 | |
| | yl)-2-((4- | (m, 1H), 6.28 (dd, J = | |
| | (trifluoromethyl)pyridin- | 12.2, 11.0 Hz, 1H), 5.19 | |
| | 2-yl)oxy)ethan-1-one | (s, 2H), 4.33 (d, J = | |
| | | 13.2 Hz, 1H), 3.97-3.80 | |
| | | (2H), 3.55 (dd, J = 17.2, | |
| | | 10.9 Hz, 1H), 3.38 (tt, | |
| | | J = 11.6, 3.8 Hz, 1H), 3.24 | |
| | | (t, J = 11.6 Hz, 1H), 2.79 | |
| | | (t, J = 14.2 Hz, 1H), | |
| | | 2.04-2.12 (m, 2H), 1.76- | |
| | | 1.85 (m, 1H), 1.49-1.58 | |
| | | (m, 1H) | |
| 45 | 1-(4-(4-(5-(2,6- | ¹H-NMR (400 MHz, | 585.4 |
| | dichlorophenyl)- | DMSO-d₆) δ 8.38 (d, J = | |
| | 4,5-dihydroisoxazol- | 5.1 Hz, 1H), 8.02 (s, 1H), | |
| | 3-yl)thiazol- | 7.55 (d, J = 0.7 Hz, | |
| | 2-yl)piperidin-1- | 1H), 7.53 (s, 1H), 7.43 | |
| | yl)-2-((4- | (dd, J = 8.6, 7.3 Hz, 1H), | |
| | (trifluoromethyl)pyridin- | 7.30 (d, J = 5.4 Hz, 1H), | |
| | 2-yl)oxy)ethan-1-one | 7.27 (s, 1H), 6.32 (dd, J = | |
| | | 12.2, 11.0 Hz, 1H), | |
| | | 5.19 (s, 2H), 4.33 (d, J = | |
| | | 13.2 Hz, 1H), 3.97-3.77 | |
| | | (2H), 3.56 (dd, J = 17.1, | |
| | | 11.0 Hz, 1H), 3.38 (tt, | |
| | | J = 11.5, 3.8 Hz, 1H), 3.24 | |
| | | (t, J = 11.9 Hz, 1H), 2.79 | |
| | | (t, J = 11.6 Hz, 1H), | |
| | | 2.04-2.12 (m, 2H), 1.76- | |
| | | 1.85 (m, 1H), 1.49-1.58 | |
| | | (m, 1H) | |
| 46 | 3-chloro-2-(3- | ¹H-NMR (400 MHz, | 645 |
| | (2-(1-(2-((4- | DMSO-d₆) δ 8.38 (d, J = | |
| | (trifluoromethyl)pyridin- | 5.4 Hz, 1H), 8.04 (s, 1H), | |
| | 2- | 7.56 (dd, J = 7.9, 1.8 | |
| | yl)oxy)acetyl)piperidin- | Hz, 1H), 7.53 (t, J = 7.8 | |
| | 4-yl)thiazol-4-yl)-4,5- | Hz, 1H), 7.47 (dd, J = | |
| | dihydroisoxazol- | 7.7, 1.8 Hz, 1H), 7.30 (dd, | |
| | 5-yl)phenyl | J = 5.3, 1.1 Hz, 1H), | |
| | methanesulfonate | 7.26-7.28 (m, 1H), 6.15 | |
| | | (dd, J = 12.2, 10.8 Hz, | |
| | | 1H), 5.19 (s, 2H), 4.33 | |
| | | (d, J = 12.7 Hz, 1H), 3.91 | |
| | | (d, J = 13.7 Hz, 1H), 3.82 | |
| | | (dd, J = 17.2, 12.3 Hz, | |
| | | 1H), 3.55 (dd, J = 17.2, | |
| | | 10.9 Hz, 4H), 3.38 (tt, J = | |
| | | 11.5, 3.7 Hz, 1H), 3.24 | |
| | | (t, J = 11.9 Hz, 1H), 2.76- | |
| | | 2.81 (m, 1H), 2.08 (t, | |
| | | J = 15.4 Hz, 2H), 1.76-1.85 | |
| | | (m, 1H), 1.48-1.58 (m, 1H) | |
| 47 | 1-(4-(4-(5-(2,6- | ¹H-NMR (400 MHz, | 585 |
| | dichlorophenyl)- | DMSO-d₆) δ 8.54 (d, J = 1.0 | |
| | 4,5-dihydroisoxazol- | Hz, 1H), 8.09 (dd, J = 8.8, | |
| | 3-yl)thiazol- | 2.7 Hz, 1H), 8.04 (s, | |
| | 2-yl)piperidin- | 1H), 7.55-7.57 (m, 2H), | |
| | 1-yl)-2-((5- | 7.45 (dd, J = 8.8, 7.3 Hz, | |
| | (trifluoromethyl)pyridin- | 1H) 7.10 (d, J = 8.8 Hz, | |
| | 2-yl)oxy)ethan-1-one | 1H), 6.34 (dd, J = 12.3, | |
| | | 11.1 Hz, 1H), 5.22 (s, | |
| | | 2H), 4.33-4.36 (m, 1H), | |
| | | 3.84-3.93 (m, 2H), 3.58 | |
| | | (dd, J = 17.2, 11.1 Hz, | |
| | | 1H), 3.38-3.43 (m, 1H), | |
| | | 3.25 (t, J = 11.7 Hz, 1H), | |
| | | 2.81 (t, J = 11.6 Hz, 1H), | |
| | | 2.10 (t, J = 15.2 Hz, | |
| | | 2H), 1.83 (t, J = 12.0 Hz, | |
| | | 1H), 1.57 (t, J = 11.5 | |
| | | Hz, 1H) | |
| 48 | 3-chloro-2-(3- | ¹H-NMR (400 MHz, | 645.1 |
| | (2-(1-(2-((5- | DMSO-d₆) δ 8.54 (s, 1H), | |
| | (trifluoromethyl)pyridin- | 8.08 (t, J = 8.2 Hz, 2H), | |
| | 2- | 7.47-7.59 (m, 3H), 7.10 | |
| | yl)oxy)acetyl)piperidin- | (d, J = 8.8 Hz, 1H), 6.17 | |
| | 4-yl)thiazol-4-yl)-4,5- | (t, J = 11.5 Hz, 1H), 5.22 | |
| | dihydroisoxazol-5- | (s, 2H), 4.34 (d, J = 12.2 | |
| | yl)phenyl | Hz, 1H), 3.79-3.93 (m, | |
| | methanesulfonate | 2H), 3.54-3.63 (m, 4H), | |
| | | 3.39-3.45 (m, 1H), 3.22- | |
| | | 3.28 (m, 1H), 2.81 (t, J = | |
| | | 11.9 Hz, 1H), 2.10 (t, J = | |
| | | 14.9 Hz, 2H), 1.79-1.82 | |
| | | (m, 1H), 1.55 (d, J = | |
| | | 11.7 Hz, 1H) | |
| 49 | 1-(4-(4-(5-(2,6- | ¹H-NMR (400 MHz, | 553.15 |
| | difluorophenyl)- | DMSO-d₆) δ 8.54 (q, J = 0.8 | |
| | 4,5-dihydroisoxazol- | Hz, 1H), 8.08-8.10 (m, | |
| | 3-yl)thiazol- | 1H), 8.04 (s, 1H), 7.47- | |
| | 2-yl)piperidin- | 7.54 (m, 1H), 7.09-7.20 | |
| | 1-yl)-2-((5- | (m, 3H), 6.01 (dd, J = | |
| | (trifluoromethyl)pyridin- | 12.1, 8.7 Hz, 1H), 5.22 | |
| | 2-yl)oxy)ethan-1-one | (s, 2H), 4.35 (d, J = 13.2 | |
| | | Hz, 1H), 3.87-3.95 (m, 2H), | |
| | | 3.54 (q, J = 8.6 Hz, | |
| | | 1H), 3.38-3.43 (m, 1H), | |
| | | 3.25 (t, J = 12.0 Hz, 1H), | |
| | | 2.68-2.83 (m, 1H), 2.10 (t, | |
| | | J = 15.3 Hz, 2H), 1.82 | |
| | | (q, J = 12.0 Hz, 1H), | |
| | | 1.51-1.60 (m, 1H) | |
| 50 | 1-(4-(4-(5-(2,4,6- | ¹H-NMR (400 MHz, | 620.95 |
| | trichlorophenyl)- | DMSO-d₆) δ 8.54 (q, J = 0.8 | |
| | 4,5-dihydroisoxazol- | Hz, 1H), 8.08-8.11 (m, 1H), | |
| | 3-yl)thiazol- | 8.04 (s, 1H), 7.78 (d, | |
| | 2-yl)piperidin- | J = 0.0 Hz, 2H), 7.10 (t, | |
| | 1-yl)-2-((5- | J = 4.4 Hz, 1H), 6.30 (dd, | |
| | (trifluoromethyl)pyridin- | J = 12.2, 11.0 Hz, 1H), | |
| | 2-yl)oxy)ethan-1-one | 5.22 (s, 2H), 4.34 (d, J = | |
| | | 13.0 Hz, 1H), 3.88 (dd, | |
| | | J = 17.4, 12.2 Hz, 2H), | |
| | | 3.57 (dd, J = 17.2, 10.9 | |
| | | Hz, 1H), 3.37-3.43 (m, | |
| | | 1H), 3.25 (dd, J = 12.1, | |
| | | 11.6 Hz, 1H), 2.68-2.83 | |
| | | (m, 1H), 2.10 (t, J = | |
| | | 15.2 Hz, 2H), 1.83 (t, J = | |
| | | 12.2 Hz, 1H), 1.57 (t, | |
| | | J = 11.5 Hz, 1H) | |
| 51 | 2-((3-bromo-5- | 1H-NMR (400 MHz, | 665 |
| | (trifluoromethyl)pyridin- | DMSO-D6) δ 8.50-8.53 (m, | |
| | 2-yl)oxy)- | 2H), 8.02 (s, 1H), 7.54 | |
| | 1-(4-(4-(5-(2,6- | (d, J = 7.8 Hz, 2H), 7.43 | |
| | dichlorophenyl)- | (dd, J = 8.7, 7.3 Hz, 1H), | |
| | 4,5-dihydroisoxazol- | 6.32 (dd, J = 12.2, 11.2 | |
| | 3-yl)thiazol- | Hz, 1H), 5.31 (s, 2H), 4.31 | |
| | 2-yl)piperidin- | (d, J = 13.1 Hz, 1H), | |
| | 1-yl)ethan-1-one | 3.86 (dd, J = 17.3, 12.4 | |
| | | Hz, 2H), 3.56 (dd, J = | |
| | | 17.1, 11.0 Hz, 1H), 3.38 (tt, | |
| | | J = 11.4, 3.7 Hz, 1H), | |
| | | 3.21-3.27 (m, 1H), 2.77- | |
| | | 2.83 (m, 1H), 2.05-2.12 | |
| | | (m, 2H), 1.82 (dd, J = 23.8, | |
| | | 11.7 Hz, 1H), 1.55 (q, | |
| | | J = 11.1 Hz, 1H) | |
| 52 | 2-(3-(2-(1-(2- | 1H-NMR (400 MHz, | 725 |
| | ((3-bromo-5- | DMSO-D6) δ 8.54 (t, J = | |
| | (trifluoromethyl)pyridin- | 0.9 Hz, 1H), 8.50 (d, J = | |
| | 2- | 1.8 Hz, 1H), 8.04 (s, 1H), | |
| | yl)oxy)acetyl)piperidin- | 7.51-7.59 (m, 2H), 7.47 | |
| | 4-yl)thiazol-4-yl)-4,5- | (dd, J = 7.8, 1.8 Hz, 1H), | |
| | dihydroisoxazol-5-yl)-3- | 6.15 (dd, J = 12.2, 11.1 Hz, | |
| | chlorophenyl | 1H), 5.31 (s, 2H), 4.31 | |
| | methanesulfonate | (d, J = 13.0 Hz, 1H), | |
| | | 3.78-3.90 (m, 2H), 3.52-3.58 | |
| | | (m, 4H), 3.38 (qd, J = 7.6, | |
| | | 3.7 Hz, 1H), 3.21-3.27 | |
| | | (m, 1H), 2.77-2.83 (m, | |
| | | 1H), 2.05-2.12 (m, 2H), | |
| | | 1.81 (d, J = 12.2 Hz, 1H), | |
| | | 1.54 (d, J = 11.8 Hz, | |
| | | 1H) | |
| 53 | 2-((3-bromo-5- | 1H-NMR (400 MHz, | 631.05 |
| | (trifluoromethyl)pyridin- | DMSO-D6) δ 8.50-8.54 (m, | |
| | 2-yl)oxy)- | 2H), 8.02 (s, 1H), 7.45-7.53 | |
| | 1-(4-(4-(5-(2,6- | (m, 1H), 7.12-7.18 | |
| | difluorophenyl)- | (m, 2H), 6.00 (dd, J = 12.1, | |
| | 4,5-dihydrolsoxazol- | 8.6 Hz, 1H), 5.31 (s, | |
| | 3-yl)thiazol- | 2H), 4.31 (d, J = 12.8 Hz, | |
| | 2-yl)piperidin- | 1H), 3.86-3.93 (m, 2H), | |
| | 1-yl)ethan-1-one | 3.53 (q, J = 8.6 Hz, 1H), | |
| | | 3.38 (tt, J = 11.5, 3.7 Hz, | |
| | | 1H), 3.21-3.27 (m, 1H), | |
| | | 2.80 (t, J = 11.8 Hz, 1H), | |
| | | 2.05-2.12 (m, 2H), 1.82 | |
| | | (d, J = 11.9 Hz, 1H), 1.55 | |
| | | (d, J = 11.6 Hz, 1H) | |
| 54 | 2-((3-bromo-5- | 1H-NMR (400 MHz, | 698.95 |
| | (trifluoromethyl)pyridin- | DMSO-D6) δ 8.50-8.54 (m, | |
| | 2-yl)oxy)- | 2H), 8.03 (s, 1H), 7.76 | |
| | 1-(4-(4-(5-(2,4,6- | (s, 2H), 6.28 (dd, J = 12.2, | |
| | trichlorophenyl)- | 11.2 Hz, 1H), 5.31 (s, 2H), | |
| | 4,5-dihydroisoxazol- | 4.31 (d, J = 12.8 Hz, | |
| | 3-yl)thiazol- | 1H), 3.86 (dd, J = 17.3, | |
| | 2-yl)piperidin- | 12.4 Hz, 2H), 3.55 (dd, J = | |
| | 1-yl)ethan-1-one | 17.3, 10.9 Hz, 1H), 3.38 | |
| | | (tt, J = 11.4, 3.6 Hz, | |
| | | 1H), 3.21-3.27 (m, 1H), | |
| | | 2.77-2.82 (m, 1H), 2.04- | |
| | | 2.12 (m, 2H), 1.77-1.86 | |
| | | (m, 1H), 1.54 (dd, J = | |
| | | 22.4, 10.9 Hz, 1H) | |
| 55 | 1-(4-(4-(5-(2,6- | 1H-NMR (400 MHz, | 601.1 |
| | dichlorophenyl)- | DMSO-D6) δ 8.76 (d, J = | |
| | 4,5-dihydroisoxazol- | 1.1 Hz, 1H), 7.99-8.02 | |
| | 3-yl)thiazol- | (m, 2H), 7.53-7.60 (m, | |
| | 2-yl)piperidin- | 3H), 7.43 (dd, J = 8.9, | |
| | 1-yl)-2-((5- | 7.3 Hz, 1H), 6.32 (dd, J = | |
| | (trifluoromethyl)pyridin- | 12.2 11.2 Hz, 1H), 4.40 | |
| | 2-yl)thio)ethan-1-one | (d, J = 13.3 Hz, 1H), 4.31 | |
| | | (dd, J = 24.2, 15.4 Hz, 2H), | |
| | | 4.10 (d, J = 13.8 Hz, | |
| | | 1H), 3.86 (dd, J = 17.3, | |
| | | 12.4 Hz, 1H), 3.56 (dd, J = | |
| | | 17.2, 11.1 Hz, 1H), 3.38 | |
| | | (tt, J = 11.4, 3.8 Hz, | |
| | | 1H), 3.28 (d, J = 11.6 Hz, | |
| | | 1H), 2.79-2.85 (m, 1H), | |
| | | 2.06-2.14 (m, 2H), 1.81 | |
| | | (d, J = 12.1 Hz, 1H), | |
| | | 1.53-1.56 (m, 1H) | |
| 56 | 3-chloro-2-(3- | 1H-NMR (400 MHz, | 661.1 |
| | (2-(1-(2-((5- | DMSO-D6) δ 8.76 (t, J = | |
| | (trifluoromethyl)pyridin- | 1.1 Hz, 1H), 7.99-8.04 (m, | |
| | 2- | 2H), 7.51-7.60 (m, 3H), | |
| | yl)thio)acetyl)piperidin- | 7.47 (dd, J = 7.7, 1.8 Hz, | |
| | 4-yl)thiazol-4-yl)-4,5- | 1H), 6.15 (dd, J = 12.2, | |
| | dihydroisoxazol- | 11.1 Hz, 1H), 4.40 (d, | |
| | 5-yl)phenyl | J = 13.0 Hz, 1H), 4.31 (dd, | |
| | methanesulfonate | J = 20.9, 15.5 Hz, 2H), | |
| | | 4.09 (d, J = 13.6 Hz, 1H), | |
| | | 3.81 (dd, J = 17.3, 12.4 Hz, | |
| | | 1H), 3.51-3.58 (m, | |
| | | 4H), 3.34-3.41 (m, 1H), | |
| | | 3.27 (d, J = 11.8 Hz, 1H), | |
| | | 2.79-2.85 (m, 1H), 2.07- | |
| | | 2.14 (m, 2H), 1.80 (d, J = | |
| | | 12.2 Hz, 1H), 1.55 (d, | |
| | | J = 11.6 Hz, 1H) | |
| 57 | 1-(4-(4-(5-(2,6- | 1H-NMR (400 MHz, | 569.1 |
| | difluorophenyl)- | DMSO-D6) δ 8.76 (t, J = | |
| | 4,5-dihydroisoxazol- | 1.1 Hz, 1H), 7.99-8.04 (m, | |
| | 3-yl)thiazol- | 2H), 7.51-7.60 (m, 3H), | |
| | 2-yl)piperidin- | 7.47 (dd, J = 7.7, 1.8 Hz, | |
| | 1-yl)-2-((5- | 1H), 6.15 (dd, J = 12.2, | |
| | (trifluoromethyl)pyridin- | 11.1 Hz, 1H), 4.40 (d, | |
| | 2-yl)thio)ethan-1-one | J = 13.0 Hz, 1H), 4.31 (dd, | |
| | | J = 20.9, 15.5 Hz, 2H), | |
| | | 4.09 (d, J = 13.6 Hz, 1H), | |
| | | 3.81 (dd, J = 17.3, 12.4 | |
| | | Hz, 1H), 3.51-3.58 (m, | |
| | | 4H), 3.34-3.41 (m, 1H), | |
| | | 3.27 (d, J = 11.8 Hz, 1H), | |
| | | 2.79-2.85 (m, 1H), 2.07- | |
| | | 2.14 (m, 2H), 1.80 (d, J = | |
| | | 12.2 Hz, 1H), 1.55 | |
| | | (d, J = 11.6 Hz, 1H) | |
| 58 | 1-(4-(4-(5-(2,4,6- | 1H-NMR (400 MHz, | 636.95 |
| | trichlorophenyl)- | DMSO-D6) δ 8.76 | |
| | 4,5-dihydroisoxazol- | (t, J = 1.1 Hz, 1H), | |
| | 3-yl)thiazol- | 7.99-8.03 (m, 2H), | |
| | 2-yl)piperidin- | 7.76 (s, 2H), 7.59 (d, | |
| | 1-yl)-2-((5- | J = 8.6 Hz, 1H), 6.28 (dd, | |
| | (trifluoromethyl)pyridin- | J = 12.3, 11.1 Hz, 1H), | |
| | 2-yl)thio)ethan-1-one | 4.40 (d, J = 13.0 Hz, 1H), | |
| | | 4.31 (dd, J = 24.3, 15.4 | |
| | | Hz, 2H), 4.10 (d, J = 13.8 | |
| | | Hz, 1H), 3.86 (dd, J = | |
| | | 17.3, 12.4 Hz, 1H), 3.55 | |
| | | (dd, J = 17.3, 10.9 Hz, | |
| | | 1H), 3.38 (tt, J = 11.5, | |
| | | 3.7 Hz, 1H), 3.27 (d, J = | |
| | | 11.8 Hz, 1H), 2.79-2.84 | |
| | | (m, 1H), 2.05-2.14 (m, | |
| | | 2H), 1.76-1.85 (m, 1H), | |
| | | 1.55 (dd, J = 21.7, 12.1 | |
| | | Hz, 1H) | |
| 59 | 1-(4-(4-(1,5- | ¹H-NMR (400 MHz, | 520.15 |
| | dihydrobenzo[e][1, | DMSO-d6) δ 8.38 (d, J = | |
| | 3]dioxepin-3- | 4.3 Hz, 1H), 8.09-8.11 (m, | |
| | yl)thiazol-2- | 1H), 7.57 (s, 1H), 7.25 (t, | |
| | yl)piperidin-1-yl)-2- | J = 9.2 Hz, 4H), 7.16 (dd, | |
| | ((3- | J = 7.3, 4.9 Hz, 1H), | |
| | (trifluoromethyl)pyridin- | 6.03 (s, 1H), 5.26 (s, 2H), | |
| | 2-yl)oxy)ethan-1-one | 5.01 (d, J = 14.7 Hz, | |
| | | 2H), 4.91-4.95 (m, 2H), | |
| | | 4.31 (d, J = 12.8 Hz, 1H), | |
| | | 3.89 (d, J = 13.4 Hz, 1H), | |
| | | 3.19-3.29 (m, 2H), 2.77 | |
| | | (t, J = 11.9 Hz, 1H), | |
| | | 2.02-2.08 (m, 2H), 1.78 (dd, | |
| | | J = 20.8, 12.2 Hz, 1H), | |
| | | 1.51 (dd, J = 20.5, 11.9 | |
| | | Hz, 1H) | |
| 60 | 3-chloro-2-(3- | 1H-NMR (400 MHz, | 600.9 |
| | (2-(1-(2-((3- | DMSO-D6) δ 8.74 (d, J = | |
| | (trifluoromethyl)pyridin- | 4.9 Hz, 1H), 8.08 (d, J = | |
| | 2- | 7.3 Hz, 1H), 8.01 (s, 1H), | |
| | yl)thio)acetyl)piperidin- | 7.54 (d, J = 7.9 Hz, 2H), | |
| | 4- | 7.43 (dd, J = 8.6, 7.3 Hz, | |
| | yl)thiazol-4-yl)-4,5- | 1H), 7.31 (q, J = 4.3 Hz, | |
| | dihydroisoxazol- | 1H), 6.33 (t, J = 11.9 Hz, | |
| | 5-yl)phenyl | 1H), 4.38 (d, J = 15.9 Hz, | |
| | methanesulfonate | 2H), 4.25 (d, J = 15.9 | |
| | | Hz, 1H), 4.12 (d, J = 14.1 | |
| | | Hz, 1H), 3.87 (dd, J = | |
| | | 17.1, 12.2 Hz, 1H), 3.56 | |
| | | (dd, J = 17.1, 11.0 Hz, | |
| | | 1H), 3.39 (qd, J = 7.7, 3.7 | |
| | | Hz, 1H), 3.28 (d, J = | |
| | | 12.2 Hz, 1H), 2.81 (t, | |
| | | J = 11.6 Hz, 1H), 2.08 (dd, | |
| | | J = 22.0, 12.8 Hz, 2H), | |
| | | 1.87 (q, J = 12.0 Hz, 1H), | |
| | | 1.51-1.60 (m, 1H) | |
| 61 | 3-chloro-2-(3- | 1H-NMR (400 MHz, | 660.9 |
| | (2-(1-(2-((3- | DMSO-D6) δ 8.74 (d, J = | |
| | (trifluoromethyl)pyridin- | 4.9 Hz, 1H), 8.08 (d, J = | |
| | 2- | 8.6 Hz, 1H), 8.03 (s, 1H), | |
| | yl)thio)acetyl)piperidin- | 7.51-7.58 (m, 2H), 7.47 | |
| | 4- | (dd, J = 7.3, 1.8 Hz, 1H), | |
| | yl)thiazol-4-yl)-4,5- | 7.31 (q, J = 4.1 Hz, 1H), | |
| | dihydroisoxazol- | 6.16 (t, J = 11.6 Hz, 1H), | |
| | 5-yl)phenyl | 4.39 (d, J = 15.9 Hz, 2H), | |
| | methanesulfonate | 4.23-4.27 (m, 1H), 4.12 | |
| | | (d, J = 13.4 Hz, 1H), | |
| | | 3.82 (dd, J = 17.1, 12.2 Hz, | |
| | | 1H), 3.52-3.60 (m, 4H), | |
| | | 3.37-3.42 (m, 1H), 3.24- | |
| | | 3.27 (m, 1H), 2.81 (t, J = | |
| | | 11.6 Hz, 1H), 2.07 (dd, J = | |
| | | 24.1, 10.7 Hz, 2H), | |
| | | 1.86-1.92 (m, 1H), 1.50- | |
| | | 1.56 (m, 1H) | |
| 62 | 1-(4-(4-(5-(2,6- | 1H-NMR (400 MHz, | 569.35 |
| | difluorophenyl)- | DMSO-D6) δ 8.74 (d, J = | |
| | 4,5-dihydroisoxazol- | 4.3 Hz, 1H), 8.09 (t, J = | |
| | 3-yl)thiazol- | 7.6 Hz, 1H), 8.01 (s, 1H), | |
| | 2-yl)piperidin- | 7.45-7.53 (m, 1H), 7.31 | |
| | 1-yl)-2-((3- | (q, J = 4.3 Hz, 1H), 7.12- | |
| | (trifluoromethyl)pyridin- | 7.18 (m, 2H) 6.00 (dd, | |
| | 2- | J = 12.2, 8.6 Hz, 1H), 4.39 | |
| | yl)thio)ethan-1-one | (d, J = 15.3 Hz, 2H), | |
| | | 4.25 (t, J = 7.6 | |
| | | Hz, 1H), 4.12 | |
| | | (d, J = 14.1 Hz, 1H), 3.90 | |
| | | (dd, J = 17.1, 12.2 Hz, | |
| | | 1H), 3.53 (q, J = 8.8 Hz, | |
| | | 1H), 3.40 (tt, J = 11.5, | |
| | | 3.8 Hz, 1H), 3.28 (d, J = | |
| | | 12.2 Hz, 1H), 2.81 (t, J = | |
| | | 11.3 Hz, 1H), 2.07 (dd, | |
| | | J = 22.9, 12.5 Hz, 2H), | |
| | | 1.87 (q, J = 11.8 Hz, | |
| | | 1H), 1.52-1.60 (m, 1H) | |
| 63 | 1-(4-(4-(5-(2,4,6- | 1H-NMR (400 MHz, | 634.9 |
| | trichlorophenyl)- | DMSO-D6) δ 8.74 (d, J = | |
| | 4,5-dihydroisoxazol- | 4.3 Hz, 1H), 8.08 (d, J = | |
| | 3-yl)thiazol- | 7.3 Hz, 1H), 8.02 (s, 1H), | |
| | 2-yl)piperidin- | 7.76 (s, 2H), 7.31 (dd, | |
| | 1-yl)-2-((3- | J = 7.5, 5.0 Hz, 1H), 6.29 | |
| | (trifluoromethyl)pyridin- | (t, J = 11.6 Hz, 1H) 4.38 | |
| | 2- | (d, J = 15.9 Hz, 2H), | |
| | yl)thio)ethan-1-one | 4.25 (d, J = 15.3 Hz, 1H), | |
| | | 4.12 (d, J = 13.4 Hz, | |
| | | 1H), 3.87 (dd, J = 17.1, | |
| | | 12.2 Hz, 1H), 3.55 (dd, J = | |
| | | 17.1, 11.0 Hz, 1H), 3.39 | |
| | | (qd, J = 7.7, 3.7 Hz, | |
| | | 1H), 3.28 (d, J = 12.2 | |
| | | Hz, 1H), 2.81 (t, J = 11.6 | |
| | | Hz, 1H), 2.07 (dd, J = 22.3, | |
| | | 13.1 Hz, 2H), 1.88 (t, | |
| | | J = 12.2 Hz, 1H), | |
| | | 1.54-1.60 (m, 1H) | |
| 64 | (trifluoromethyl)phenyl)- | 1H-NMR (400 MHz, | 585.15 |
| | 4,5-dihydroisoxazol- | DMSO-D6) δ 8.38-8.39 (m, | |
| | 3-yl)thiazol-2- | 1H), 8.09-8.11 (m, 1H), | |
| | yl)piperidin- | 8.03 (s, 1H), 7.72 (t, J = | |
| | 1-yl)-2-((3- | 7.9 Hz, 3H), 7.64 (t, | |
| | (trifluoromethyl)pyridin- | J = 7.6 Hz, 1H), 7.16 (dd, | |
| | 2- | J = 7.6, 5.2 Hz, 1H), 5.87 | |
| | yl)oxy)ethan-1-one | (dd, J = 11.0,7.9 Hz, 1H), | |
| | | 5.26 (s, 2H), 4.32 (d, J = | |
| | | 12.8 Hz, 1H), 3.93 (dd, J = | |
| | | 17.1, 11.0 Hz, 2H), 3.34- | |
| | | 3.45 (m, 2H), 3.23 (t, J = | |
| | | 11.9 Hz, 1H), 2.78 (t, | |
| | | J = 11.9 Hz, 1H), 2.06 (t, | |
| | | J = 13.4 Hz, 2H), 1.80 (dd, | |
| | | J = 21.1, 11.9 Hz, 1H), | |
| | | 1.49-1.57 (m, 1H) | |
| 65 | 2-((3-chloro-5- | 1H-NMR (400 MHz, | 603 |
| | (trifluoromethyl)pyridin- | DMSO-d6) δ 8.77 | |
| | 2- | (d, J = 1.2 Hz, 1H), | |
| | yl)thio)-1- | 8.35 (d, J = 1.8 Hz, | |
| | (4-(4-(5-(2,6- | 1H), 8.02 (s, 1H), 7.45- | |
| | difluorophenyl)-4,5- | 7.53 (m, 1H), 7.12-7.17 | |
| | dihydroisoxazol- | (m, 2H), 5.99 (dd, J = | |
| | 3-yl)thiazol-2- | 12.2, 8.6 Hz, 1H), 4.29- | |
| | yl)piperidin-1- | 4.41 (m, 3H), 4.10 (d, J = | |
| | yl)ethan-1-one | 13.4 Hz, 1H), 3.89 (dd, | |
| | | J = 17.1, 12.2 Hz, 1H), | |
| | | 3.52 (q, J = 8.6 Hz, 1H), | |
| | | 3.34-3.42 (m, 1H), 3.28 | |
| | | (s, 1H), 2.83 (t, J = 11.3 Hz, | |
| | | 1H), 2.10 (q, J = 12.4 | |
| | | Hz, 2H), 1.83 (d, J = 12.8 | |
| | | Hz, 1H), 1.56 (d, J = | |
| | | 12.2 Hz, 1H) | |
| 66 | 1-(4-(4-(5-(2- | 1H-NMR (400 MHz, | 535.55 |
| | fluorophenyl)-4,5- | DMSO-D6) δ 8.38-8.40 (m, | |
| | dihydroisoxazol-3- | 1H), 8.10-8.12 (m, 1H), | |
| | yl)thiazol-2- | 8.04 (s, 1H), 7.38-7.47 | |
| | yl)piperidin-1-yl)-2-((3- | (m, 2H), 7.21-7.28 (m, | |
| | (trifluoromethyl)pyridin- | 2H), 7.16 (dd, J = 7.3, 5.5 | |
| | 2- | Hz, 1H), 5.91 (dd, J = 11.3, | |
| | yl)oxy)ethan-1-one | 7.6 Hz, 1H), 5.26 (s, | |
| | | 2H), 4.32 (d, J = 12.8 Hz, | |
| | | 1H), 3.92 (dd, J = 17.1, | |
| | | 11.0 Hz, 2H), 3.35-3.44 | |
| | | (m, 2H), 3.23 (t, J = 12.2 | |
| | | Hz, 1H), 2.78 (t, J = 11.9 | |
| | | Hz, 1H), 2.06 (t, J = | |
| | | 13.1 Hz, 2H), 1.81 (q, | |
| | | J = 10.8 Hz, 1H), 1.54 (dd, | |
| | | J = 20.8, 11.6 Hz, 1H) | |
| 67 | 2-((5-chloro-3- | 1H-NMR (400 MHz, | 581.05 |
| | (trifluoromethyl)pyridin- | DMSO-D6) δ 8.47 (d, J = | |
| | 2-yl)oxy)- | 2.4 Hz, 1H), 8.28 (d, J = | |
| | 1-(4-(4-(5-(4- | 2.8 Hz, 1H), 8.00 (s, 1H), | |
| | methoxyl)henyl)-4,5- | 7.30-7.33 (m, 2H), 6.92- | |
| | dihydroisoxazol- | 6.96 (m, 2H), 5.65 (dd, J = | |
| | 3-yl)thiazol-2- | 11.0, 8.6 Hz, 1H), 5.28 | |
| | yl)piperidm-1- | (s, 2H), 4.30 (d, J = 13.4 | |
| | yl)ethan-1-one | Hz, 1H), 3.77-3.88 (m, | |
| | | 2H), 3.74 (s, 3H), 3.32- | |
| | | 3.39 (m, 2H), 3.22 (t, | |
| | | J = 11.9 Hz, 1H), 2.78 (t, | |
| | | J = 11.6 Hz, 1H), 2.06 (t, | |
| | | J = 11.9 Hz, 2H), 1.80 (d, | |
| | | J = 11.0 Hz, 1H), 1.53 (d, | |
| | | J = 8.6 Hz, 1H) | |
| 68 | 2-((3-chloro-5- | 1H-NMR (400 MHz, | 670.9 |
| | (trifluoromethyl)pyridin- | DMSO-d6) δ | |
| | 2-yl)thio)-1-(4- | 8.78 (q, J = 1.0 | |
| | (4-(5-(2,4,6- | Hz, 1H), 8.37 (d, J = 1.5 | |
| | trichlorophenyl)-4,5- | Hz, 1H), 8.05 (s, 1H), | |
| | dihydroisoxazol- | 7.78 (s, 2H), 6.30 (dd, | |
| | 3-yl)thiazol-2- | J = 12.2, 11.0 Hz, 1H), | |
| | yl)piperidin-1- | 4.34-4.41 (m, 3H), 4.22- | |
| | yl)ethan-1-one | 4.00 (1H), 3.88 (dd, J = | |
| | | 17.2, 12.3 Hz, 1H), 3.56 | |
| | | (dd, J = 17.1, 11.0 Hz, | |
| | | 1H), 3.37 (d, J = 29.6 Hz, | |
| | | 2H), 2.84 (s, 1H), 2.22- | |
| | | 2.03 (2H), 1.94-1.78 (1H), | |
| | | 1.68-1.50 (1H) | |
| 69 | N-(((2-(4-(4-(5-(2,6- | 1H-NMR (400 MHz, | 650.6 |
| | difluorophenyl)-4,5- | DMSO-D6) δ 8.76 (s, 1H), | |
| | dihydroisoxazol- | 8.27 (dd, J = 8.9, 2.3 Hz, | |
| | 3-yl)thiazol-2- | 1H), 8.02-8.05 (m, 1H), | |
| | yl)piperidin-1-yl)- | 7.47-7.55 (m, 1H), 7.14- | |
| | 2-oxoethyl)(5- | 7.21 (m, 3H), 6.03 (dd, J = | |
| | (trifluoromethyl)pyridin- | 12.2, 8.6 Hz, 1H), 4.88 | |
| | 2-yl)amino)(dimethyl- | (dd, J = 23.6, 17.2 Hz, | |
| | amino)methylene)-N- | 2H), 4.40 (d, J = 13.0 Hz, | |
| | methylmethanaminium | 1H), 3.88-4.12 (m, 2H), | |
| | | 3.41-3.64 (m, 2H), 3.31 | |
| | | (s, 1H), 3.15 (d, J = 18.6 | |
| | | Hz, 6H), 2.86-2.95 (m, | |
| | | 7H), 2.08-2.21 (m, 2H), | |
| | | 1.87-1.96 (m, 1H), 1.63 | |
| | | (qd, J = 12.1, 3.6 Hz, 1H) | |
| 70 | 1-(4-(4-(5-(2,6- | 1H-NMR (400 MHz, | 585.05 |
| | dichlorophenyl)- | DMSO-D6) δ 8.04-8.06 (m, | |
| | 4,5-dihydroisoxazol- | 1H), 7.96-8.01 (m, 1H), | |
| | 3-yl)thiazol- | 7.55-7.57 (m, 2H), 7.43- | |
| | 2-yl)piperidin- | 7.49 (m, 2H), 7.22-7.24 | |
| | 1-yl)-2-((6- | (m, 1H), 6.31-6.39 (m, | |
| | (trifluoromethyl)pyridin- | 1H), 5.22 (d, J = 14.7 Hz, | |
| | 2- | 1H), 5.04 (d, J = 14.7 | |
| | yl)oxy)ethan-1-one | Hz, 1H), 4.36 (d, J = 12.7 | |
| | | Hz, 1H), 3.92-3.97 (m, | |
| | | 1H), 3.85-3.89 (m, 1H), | |
| | | 3.58 (dd, J = 17.1, 11.0 | |
| | | Hz, 1H), 3.45 (d, J = 3.7 | |
| | | Hz, 1H), 3.23-3.29 (m, | |
| | | 1H), 2.78-2.84 (m, 1H), | |
| | | 2.07-2.17 (m, 2H), 1.77- | |
| | | 1.83 (m, 1H), 1.53 (d, | |
| | | J = 15.9 Hz, 1H) | |
| 71 | 3-chloro-2-(3- | 1H-NMR (400 MHz, | 645.1 |
| | (2-(1-(2-((6- | DMSO-D6) δ 8.07 (d, J = | |
| | (trifluoromethyl)pyridin- | 3.9 Hz, 1H), 7.99 (t, | |
| | 2- | J = 7.9 Hz, 1H), 7.53-7.62 | |
| | yl)oxy)acetyl)piperidin- | (m, 2H), 7.43-7.50 (m, | |
| | 4- | 2H), 7.23 (d, J = 8.3 Hz, | |
| | yl)thiazol-4- | 1H), 6.17 (dd, J = 12.1, | |
| | yl)-4,5- | 10.9 Hz, 1H), 4.76-5.24 | |
| | dihydroisoxazol- | (m, 2H), 4.36 (d, J = 12.2 | |
| | 5-yl)phenyl | Hz, 1H), 3.80-3.97 (m, | |
| | methanesulfonate | 2H), 3.54-3.62 (m, 4H), | |
| | | 3.36-3.45 (m, 1H), 3.25 | |
| | | (t, J = 13.0 Hz, 1H), 2.68- | |
| | | 2.83 (m, 1H), 2.06-2.16 | |
| | | (m, 2H), 1.79-1.83 (m, | |
| | | 1H), 1.53 (s, 1H) | |
| 72 | 1-(4-(4-(5-(2,6- | 1H-NMR (400 MHz, | 553.1 |
| | difluorophenyl)- | DMSO-D6) δ 8.04-8.05 (m, | |
| | 4,5-dihydroisoxazol- | 1H), 7.96-8.01 (m, 1H), | |
| | 3-yl)thiazol- | 7.43-7.62 (m, 2H), 7.13- | |
| | 2-yl)piperidin- | 7.24 (m, 3H), 6.02 (dd, | |
| | 1-yl)-2-((6- | J = 12.2, 8.6 Hz, 1H), 5.22 | |
| | (trifluoromethyl)pyridin- | (d, J = 14.9 Hz, 1H), | |
| | 2- | 4.87-5.06 (m, 1H), 4.36 (d, | |
| | yl)oxy)ethan-1-one | J = 12.5 Hz, 1H), 3.87-4.08 | |
| | | (m, 2H), 3.55 (q, J = | |
| | | 8.6 Hz, 1H), 3.43 (td, | |
| | | J = 7.6, 3.7 Hz, 1H), 3.26 (t, | |
| | | J = 12.7 Hz, 1H), 2.80 | |
| | | (t, J = 12.1 Hz, 1H), 2.11 | |
| | | (dd, J = 27.9, 12.2 Hz, 2H), | |
| | | 1.78 (s, 1H), 1.50- | |
| | | 1.55 (m, 1H) | |
| 73 | 1-(4-(4-(5-(2,4,6- | 1H-NMR (400 MHz, | 620.95 |
| | trichlorophenyl)- | DMSO-D6) δ 8.05 (d, J = | |
| | 4,5-dihydroisoxazol- | 4.9 Hz, 1H), 7.96-8.01 | |
| | 3-yl)thiazol- | (m, 1H), 7.74-7.78 (m, | |
| | 2-yl)piperidin-1- | 2H), 7.47 (d, J = 7.3 Hz, | |
| | yl)-2-((6- | 1H), 7.23 (d, J = 8.3 Hz, | |
| | (trifluoromethyl)pyridin- | 1H), 6.30 (dd, J = 12.3, | |
| | 2- | 10.9 Hz, 1H), 5.22 (d, J = | |
| | yl)oxy)ethan-1-one | 14.7 Hz, 1H), 5.04 (d, J = | |
| | | 14.7 Hz, 1H), 4.36 (d, J = | |
| | | 12.7 Hz, 1H), 3.85-3.97 | |
| | | (m, 2H), 3.57 (dd, J = | |
| | | 17.2, 10.9 Hz, 1H), 3.41 | |
| | | (qd, J = 7.7, 3.7 Hz, 1H), | |
| | | 3.25 (t, J = 12.1 Hz, 1H), | |
| | | 2.68-2.84 (m, 1H), 2.06- | |
| | | 2.16 (m, 2H), 1.79 (t, | |
| | | J = 11.9 Hz, 1H), 1.49-1.57 | |
| | | (m, 1H) | |
| 74 | 2-((3-chloro-5- | 1H-NMR (400 MHz, | 635.1 |
| | (trifluoromethyl)pyridin- | DMSO-D6) δ 8.77 (q, J = | |
| | 2- | 1.0 Hz, 1H), 8.35-8.36 | |
| | yl)thio)-1-(4- | (m, 1H), 8.02 (s, 1H), 7.55 | |
| | (4-(5-(2,6- | (d, J = 0.7 Hz, 1H), 7.53 | |
| | dichlorophenyl)-4,5- | (s, 1H), 7.43 (dd, J = 8.8, | |
| | dihydroisoxazol- | 7.3 Hz, 1H), 6.32 (dd, | |
| | 3-yl)thiazol-2- | J = 12.2, 11.2 Hz, 1H), | |
| | yl)piperidin-1- | 4.26-4.44 (m, 3H), 4.10 | |
| | yl)ethan-1-one | (d, J = 14.2 Hz, 1H), 3.86 | |
| | | (dd, J = 17.1, 12.5 Hz, 1H), | |
| | | 3.56 (dd, J = 17.4, | |
| | | 11.0 Hz, 1H), 3.34-3.42 | |
| | | (m, 2 H), 2.83 (t, J = 11.2 | |
| | | Hz, 1H), 2.10 (q, J = 12.4 | |
| | | Hz, 2H), 1.83 (q, J = | |
| | | 12.1 Hz, 1H), 1.54-1.60 | |
| | | (m, 1H) | |
| 75 | 1-(4-(4-(5-(2,6- | 1H-NMR (400 MHz, | 584.05 |
| | dichlorophenyl)- | DMSO-D6) δ 8.31 (d, J = | |
| | 4,5-dihydroisoxazol- | 17.9 Hz, 1H), 8.08 (d, | |
| | 3-yl)thiazol- | J = 39.1 Hz, 1H), 7.65-7.73 | |
| | 2-yl)piperidin-1- | (m, 1H), 7.57 (t, J = 8.3 | |
| | yl)-2-((5- | Hz, 2H), 7.42-7.46 (m, | |
| | (trifluoromethyl)pyridin- | 2H), 6.83 (dd, J = 28.9, 9.0 | |
| | 2- | Hz, 1H), 6.33 (t, J = | |
| | yl)amino)ethan-1-one | 11.6 Hz, 1H), 4.43 (d, | |
| | | J = 13.4 Hz, 1H), 4.24 (d, | |
| | | J = 3.2 Hz, 2H), 3.84-4.00 | |
| | | (m, 2H), 3.47-3.66 (m, | |
| | | 1H), 3.24 (t, J = 12.3 Hz, | |
| | | 1H), 2.82 (t, J = 11.9 | |
| | | Hz, 1H), 2.62-2.59 (1H), | |
| | | 2.08-2.14 (m, 2H), 1.76- | |
| | | 1.82 (m, 1H), 1.57 (d, | |
| | | J = 11.0 Hz, 1H) | |
| 76 | 1-(4-(4-(5-(2,6- | 1H-NMR (400 MHz, | 531.05 |
| | dichlorophenyl)- | DMSO-D6) δ 8.03 (t, J = | |
| | 4,5-dihydroisoxazol- | 3.3 Hz, 1H), 7.93-7.95 (m, | |
| | 3-yl)thiazol- | 1H), 7.54-7.57 (m, 3H), | |
| | 2-yl)piperidin-1- | 7.45 (dd, J = 8.8, 7.3 Hz, | |
| | yl)-2-((3- | 1H), 6.88 (dd, J = 7.1, | |
| | methylpyridin-2- | 5.1 Hz, 1H) 6.34 (dd, | |
| | yl)oxy)ethan-1- | J = 12.3, 11.1 Hz, 1H) | |
| | one | 4.90-5.12, (m, 2H), 4.35- | |
| | | 4.42 (m, 1H), 3.85-3.98 | |
| | | (m, 2H), 3.58 (dd, J = | |
| | | 17.1, 11.0 Hz, 1H), 3.40 | |
| | | (qd, J = 7.7, 3.9 Hz, 1H), | |
| | | 3.25 (t, J = 12.3 Hz, | |
| | | 1H), 2.68-2.83 (m, 1H), | |
| | | 2.19 (s, 3H), 2.03-2.13 | |
| | | (m, 1H), 1.83 (t, J = 12.1 | |
| | | Hz, 1H), 1.55-1.60 (m, | |
| | | 1H) | |
| 77 | 1-(4-(4-(5-(2,6- | 1H-NMR (400 MHz, | 499.15 |
| | difluorophenyl)- | DMSO-D6) δ 8.03-8.07 (m, | |
| | 4,5-dihydroisoxazol- | 1H), 7.93-7.95 (m, 1H), | |
| | 3-yl)thiazol- | 7.47-7.56 (m, 2H), 7.14- | |
| | 2-yl)piperidin- | 7.20 (m, 2H), 6.87-6.93 | |
| | 1-yl)-2-((3- | (m, 1H), 6.02 (dd, J = | |
| | methylpyridin-2- | 12.0, 8.6 Hz, 1H), 4.92-5.12 | |
| | yl)oxy)ethan-1- | (m, 2H), 4.38 (t, J = | |
| | one | 12.2 Hz, 1H), 3.87-3.98 | |
| | | (m, 2H), 3.55 (q, J = 8.6 | |
| | | Hz, 1H), 3.40 (qd, J = 7.7, | |
| | | 3.8 Hz, 1H), 3.24 (d, J = | |
| | | 12.2 Hz, 1H), 2.67-2.90 | |
| | | (m, 1H), 2.20 (s, 3H), | |
| | | 2.03-2.13 (m, 2H), 1.83 | |
| | | (t, J = 11.7 Hz, 1H), 1.50- | |
| | | 1.60 (m, 1H) | |
| 78 | 3-chloro-2-(3- | 1H-NMR (400 MHz, | 591.05 |
| | (2-(1-(2-((3- | DMSO-D6) δ 8.05-8.08 (m, | |
| | methylpyridin-2- | 1H), 7.93-7.97 (m, 1H), | |
| | yl)oxy)acetyl)piperidin- | 7.47-7.61 (m, 4H), 6.87- | |
| | 4- | 6.93 (m, 1H), 6.17 (dd, | |
| | yl)thiazol-4-yl)-4,5- | J = 12.2, 11.0 Hz, 1H), | |
| | dihydroisoxazol- | 5.07 (d, J = 35.7 Hz, | |
| | 5-yl)phenyl | 2H), 4.36 (d, J = 12.5 Hz, | |
| | methanesulfonate | 1H), 3.71-4.08 (m, 2H), | |
| | | 3.53-3.62 (m, 4H), 3.35- | |
| | | 3.44 (m, 1H), 3.25 (t, | |
| | | J = 11.7 Hz, 1H), 2.68-2.83 | |
| | | (m, 1H), 2.06-235 (m, 6H), | |
| | | 1.81-1.86 (m, 1H), | |
| | | 1.50-1.57 (m, 1H) | |
| 79 | 2((3-methylpyridin- | 1H-NMR (400 MHz, | 566.95 |
| | 2-yl)oxy)-1- | DMSO-D6) δ 8.03-8.08 (m, | |
| | (4-(4-(5-(2,4,6- | 1H), 7.93 (dq, J = 5.0, | |
| | trichlorophenyl)- | 0.8 Hz, 1H), 7.77 (d, J = | |
| | 4,5-dihydroisoxazol- | 2.4 Hz, 2H), 7.55 (dq, | |
| | 3-yl)thiazol- | J = 7.2, 0.9 Hz, 1H), 6.88 | |
| | 2-yl)piperidin-1- | (dd, J = 7.1, 5.1 Hz, 1H), | |
| | yl)ethan-1-one | 6.30 (dd, J = 12.2, 11.1 | |
| | | Hz, 1H), 5.02-5.15 (m, | |
| | | 2H), 4.36 (d, J = 12.5 Hz, | |
| | | 1H), 3.84-3.97 (m, 2H), | |
| | | 3.57 (dd, J = 17.2, 10.9 | |
| | | Hz, 1H), 3.39 (qd, J = 7.7, | |
| | | 3.8 Hz, 1H), 3.25 (t, J = | |
| | | 12.6 Hz, 1H), 2.68-2.83 | |
| | | (m, 1H), 2.19 (s, 3H), | |
| | | 2.03-2.13 (m, 2H), 1.80- | |
| | | 1.85 (m, 1H), 1.54-1.59 | |
| | | (m, 1H) | |
| 80 | 1-(4-(2-(5-(2,6- | 1H-NMR (400 MHz, | 579.15 |
| | dichlorophenyl)- | DMSO-D6) δ 8.57-8.59 (m, | |
| | 4,5-dihydroisoxazol- | 1H), 8.41 (t, J = 4.5 Hz, | |
| | 3-yl)pyridin- | 1H), 8.11-8.14 (m, 1H), | |
| | 4-yl)piperidin- | 7.91 (d, J = 3.9 Hz, 1H), | |
| | 1-yl)-2-((3- | 7.55-7.58 (m, 2H), 7.36- | |
| | (trifluoromethyl)pyridin- | 7.47 (m, 2H), 7.19 (dd, | |
| | 2- | J = 7.5. 5.0 Hz, 1H), 6.41 | |
| | yl)oxy)ethan-1-one | (dd, J = 12.5, 11.0 Hz, 1H), | |
| | | 5.29 (dd, J = 43.4, | |
| | | 14.5 Hz, 2H), 4.44 (d, | |
| | | J = 12.5 Hz, 1H), 3.88-3.97 | |
| | | (m, 2H), 3.62 (dd, J = 17.7, | |
| | | 10.9 Hz, 1H), 3.20 (t, | |
| | | J = 11.9 Hz, 1H), 2.92-2.99 | |
| | | (m, 1H), 2.68-2.73 | |
| | | (m, 1H), 1.85 (d, J = 12.2 | |
| | | Hz, 3H), 1.52 (d, J = | |
| | | 13.0 Hz, 1H) | |
| 81 | 3-chloro-2-(3-(2- | 1H-NMR (400 MHz, | 695 |
| | (1-(2-((3-chloro-5- | DMSO-D6) δ 8.79 (q, J = | |
| | (trifluoromethyl)pyridin- | 1.0 Hz, 1H), 8.37 (dd, | |
| | 2- | J = 2.1, 0.6 Hz, 1H), 8.06 | |
| | yl)thio)acetyl)piperidin- | (s, 1H), 7.47-7.59 (m, | |
| | 4- | 3H), 6.17 (dd, J = 12.2, | |
| | yl)thiazol-4-yl)-4,5- | 11.0 Hz, 1H), 4.5(m, 3H) | |
| | dihydroisoxazol- | 4.11 (d, J = 14.4 Hz, 1H), | |
| | 5-yl)phenyl | 3.83 (dd, J = 17.4, 12.2 | |
| | methanesulfonate | Hz, 1H), 3.58 (dd, J = | |
| | | 17.1, 6.4 Hz, 1H), 3.53 | |
| | | (s, 3H), 3.36-3.45 (m, 2H), | |
| | | 2.84 (t, J = 11.5 Hz, 1H), | |
| | | 2.08-2.17 (m, 2H), 1.84 | |
| | | (d, J = 11.7 Hz, 1H), | |
| | | 1.52-1.59 (m, 1H) | |
| 82 | 1-(4-(4-(5-(2,6- | 1H-NMR (400 MHz, | 584.05 |
| | dichlorophenyl)- | DMSO-D6) δ 8.28 (d, J = | |
| | 4,5-dihydroisoxazol- | 4.9 Hz, 1H), 8.01 (s, 1H), | |
| | 3-yl)thiazol- | 7.80-7.82 (m, 1H), | |
| | 2-yl)piperidin-1- | 7.53-7.55 (m, 2H), 7.43 | |
| | yl)-2-((3- | (dd, J = 8.8, 7.3 Hz, 1H), | |
| | (trifluoromethyl)pyridin- | 6.72 (dd, J = 7.5, 5.3 Hz, | |
| | 2- | 1H), 6.44 (t, J = 4.3 Hz, | |
| | yl)amino)ethan-1-one | 1H), 6.32 (dd, J = 12.2, | |
| | | 11.0 Hz, 1H), 4.42 (d, J = | |
| | | 13.2 Hz, 1H), 4.25 (d, | |
| | | J = 4.6 Hz, 2H), 3.94 (d, J = | |
| | | 13.7 Hz, 1H), 3.86 (dd, | |
| | | J = 17.1, 12.5 Hz, 1H), | |
| | | 3.56 (dd, J = 17.4, | |
| | | 11.0 Hz, 1H), 3.35-3.42 (m, | |
| | | 1H), 3.20-3.26 (m, 1H), | |
| | | 2.81-2.87 (m, 1H), 2.10 | |
| | | (dd, J = 14.9, 12.2 Hz, 2H), | |
| | | 1.79 (t, J = 12.2 Hz, | |
| | | 1H), 1.59 (dd, J = 12.3, 8.4 | |
| | | Hz, 1H) | |
| 83 | 2-((5-methyl-3- | ¹H-NMR (400 MHz, | 633 |
| | (trifluoromethyl)pyridin- | DMSO-D6) δ 8.21 (s, 1H), | |
| | 2-yl)oxy)- | 7.95-8.05 (m, 2H), | |
| | 1-(4-(4-(5-(2,4,6- | 7.78 (s, 2H), 6.30 (dd, J = | |
| | trichlorophenyl)- | 12.2, 11.0 Hz, 1H), 5.22 | |
| | 4,5-dihydroisoxazol- | (s, 2H), 4.33 (d, J = 13.2 | |
| | 3-yl)thiazol- | Hz, 1H), 3.85-3.92 (m, | |
| | 2-yl)piperidin- | 2H), 3.56-3.53 (m, 2H), | |
| | 1-yl)ethan-1-one | 3.24 (t, J = 12.1 Hz, 1H), | |
| | | 2.76-2.82 (m, 1H), 2.27 | |
| | | (s, 3H), 2.08 (t, J = 13.6 | |
| | | Hz, 2H), 1.81-1.87 (m, | |
| | | 1H), 1.54-1.56 (m, 1H) | |
| 84 | 1-(4-(4-(5-(2,6- | ¹H-NMR (400 MHz, | 599 |
| | dichlorophenyl)- | DMSO-D6) δ 8.21 (d, J = | |
| | 4,5-dihydroisoxazol- | 4.9 Hz, 1H), 7.94-8.03 | |
| | 3-yl)thiazol- | (m, 2H), 7.60-7.65 (m, | |
| | 2-yl)piperidin-1- | 2H), 7.42-7.47 (m, 1H), | |
| | yl)-2-((5-methyl-3- | 6.31-6.37 (m, 1H), 5.22 | |
| | (trifluoromethyl)pyridin- | (s, 2H), 4.34 (d, J = 13.0 | |
| | 2- | Hz, 1H), 3.85-3.94 (m, | |
| | yl)oxy)ethan-1-one | 2H), 3.58 (dd, J = 17.1, | |
| | | 11.0 Hz, 1H), 3.36-3.42 | |
| | | (m, 1H), 3.20-3.30 (m, | |
| | | 1H), 2.84-2.74 (m, 1H), | |
| | | 2.28 (s, 3H), 2.08 (t, | |
| | | J = 13.4 Hz, 2H), 1.83-1.84 | |
| | | (m, 1H), 1.57 (s, 1H) | |
| 85 | 3-chloro-2-(3-(2- | ¹H-NMR (400 MHz, | 659 |
| | (1-(2-((5-methyl-3- | DMSO-D6) δ 8.21 (s, 1H), | |
| | (trifluoromethyl)pyridin- | 7.95-8.05 (m, 2H), 7.47- | |
| | 2- | 7.59 (m, 3H), 6.17 (dd, J = | |
| | yl)oxy)acetyl)piperidin- | 12.2, 11.0 Hz, 1H), 5.22 | |
| | 4- | (s, 2H), 4.31-4.36 (m, | |
| | yl)thiazol-4-yl)-4,5- | 1H), 3.80-3.93 (m, 2H), | |
| | dihydroisoxazol- | 3.56-3.62 (m, 1H), 3.54 | |
| | 5-yl)phenyl | (s, 3H), 3.35-3.45 (m, 1H), | |
| | methanesulfonate | 3.21-3.28 (m, 1H), | |
| | | 2.68-2.83 (m, 1H), 2.31 | |
| | | (s, 3H), 2.05-2.11 (m, | |
| | | 2H), 1.86 (d, J = 12.5 Hz, | |
| | | 1H), 1.47-1.57 (m, 1H) | |
| 86 | 2-((3- | ¹H-NMR (400 MHz, | 535 |
| | (difluoromethyl)pyridin- | DMSO-D6) δ 8.30 (d, J = | |
| | 2-yl)oxy)-1-(4- | 3.4 Hz, 1H), 7.97-8.03 | |
| | (4-(5-(2,6- | (m, 2H), 7.47-7.54 (m, | |
| | difluorophenyl)-4,5- | 1H), 7.10-7.24 (m, 4H), | |
| | dihydroisoxazol- | 6.02 (dd, J = 12.1, 8.7 | |
| | 3-yl)thiazol-2- | Hz, 1H), 5.23 (s, 2H), 4.34 | |
| | yl)piperidin- | (d, J = 13.2 Hz, 1H), | |
| | 1-yl)ethan-1-one | 3.91 (dd, J = 17.2, 12.1 Hz, | |
| | | 2H), 3.55 (q, J = 8.6 | |
| | | Hz, 1H), 3.40 (qd, J = 7.7, | |
| | | 3.9 Hz, 1H), 3.22-3.28 | |
| | | (m, 1H), 2.68-2.83 (m, 1H), | |
| | | 2.03-2.10 (m, 2H), | |
| | | 1.78-1.88 (m, 1H), | |
| | | 1.55-1.60 (m, 1H) | |
| 87 | 2((3- | ¹H-NMR (400 MHz, | 603 |
| | (difluoromethyl)pyridin- | DMSO-D6) δ 8.30 (d, J = | |
| | 2-yl)oxy)-1-(4- | 4.9 Hz, 1H), 7.96-8.04 | |
| | (4-(5-(2,4,6- | (m, 2H), 7.78 (d, J = 2.0 | |
| | trichlorophenyl)-4,5- | Hz, 2H), 7.10-7.24 (m, | |
| | dihydroisoxazol- | 2H), 6.27-6.33 (m, 1H), | |
| | 3-yl)thiazol-2- | 5.23 (s, 2H), 4.34 (d, | |
| | yl)piperidin- | J = 13.2 Hz, 1H), 3.85-3.95 | |
| | 1-yl)ethan-1-one | (m, 2H), 3.51-3.61 (m, | |
| | | 1H), 3.36-3.42 (m, 1H), | |
| | | 3.20-3.28 (m, 1H), 2.77- | |
| | | 2.83 (m, 1H), 2.05-2.13 | |
| | | (m, 2H), 1.75-1.88 (m, 1H), | |
| | | 1.48-1.57 (m, 1H) | |
| 88 | 1-(4-(4-(5-(2,6- | ¹H-NMR (400 MHz, | 567 |
| | difluorophenyl)- | DMSO-D6) δ 8.21 (s, 1H), | |
| | 4,5-dihydroisoxazol- | 7.92-8.05 (m, 2H), 7.47- | |
| | 3-yl)thiazol- | 7.54 (m, 1H), 7.15-7.24 | |
| | 2-yl)piperidin-1-yl)- | (m, 2H), 6.02 (dd, J = 11.9, | |
| | 2-((5-methyl-3- | 8.7 Hz, 1H), 5.25 (s, | |
| | (trifluoromethyl)pyridin- | 2H) 4.34 (d, J = 12.0 Hz, | |
| | 2- | 1H), 3.85-3.95 (m, 2H), | |
| | yl)oxy)ethan-1-one | 3.55 (q, J = 8.6 Hz, 1H), | |
| | | 3.35-3.42 (m, 1H), | |
| | | 3.21-3.28 (m, 1H), 2.68- | |
| | | 2.82 (m, 1H), 2.31 (s, | |
| | | 3H), 2.08-2.14 (m, 2H), | |
| | | 1.82-1.88 (m, 1H), 1.51- | |
| | | 1.57 (m, 1H) | |
| 89 | 3-chloro-2-(3- | ¹H-NMR (400 MHz, | 627 |
| | (2-(1-(2-((3- | DMSO-D6) δ 8.28-8.30 (m, | |
| | (difluoromethyl)pyridin- | 1H), 7.96-8.09 (m, 2H), | |
| | 2- | 7.47-7.62 (m, 3H), 7.09- | |
| | yl)oxy)acetyl)piperidin- | 7.24 (m, 2H), 6.14-6.20 | |
| | 4- | (m, 1H), 5.23 (s, 2H), | |
| | yl)thiazol-4-yl)-4,5- | 4.34 (d, J = 13.0 Hz, 1H), | |
| | dihydroisoxazol- | 3.80-3.95 (m, 2H), | |
| | 5-yl)phenyl | 3.53-3.62 (m, 4H), 3.36- | |
| | methanesulfonate | 3.42 (m, 1H), 3.20-3.32 | |
| | | (m, 1H), 2.77-2.83 (m, 1H), | |
| | | 2.02-2.13 (m, 2H), | |
| | | 1.76-1.85 (m, 1H), | |
| | | 1.48-1.57 (m, 1H) | |
| 90 | 1-(4-(4-(5-(2,6- | ¹H-NMR (400 MHz, | 567 |
| | dichlorophenyl)- | DMSO-D6) δ 8.30 (d, J = | |
| | 4,5-dihydroisoxazol- | 3.4 Hz, 1H), 7.97-8.04 | |
| | 3-yl)thiazol- | (m, 2H), 7.43-7.57 (m, | |
| | 2-yl)piperidin-1- | 3H), 7.10-7.24 (m, 2H), | |
| | yl)-2-((3- | 6.34 (dd, J = 12.3, 11.1 | |
| | (difluoromethyl)pyridin- | Hz, 1H), 5.23 (s, 2H), | |
| | 2- | 4.34 (d, J = 12.5 Hz, 1H), | |
| | yl)oxy)ethan-1-one | 3.85-3.95 (m, 2H), 3.52- | |
| | | 3.65 (m, 1H), 3.35-3.43 | |
| | | (m, 1H), 3.20-3.28 (m, | |
| | | 1H), 2.68-2.84 (m, 1H), | |
| | | 2.06-2.13 (m, 2H), 1.77- | |
| | | 1.87 (m, 1H), 1.50-1.62 | |
| | | (m, 1H) | |
| 91 | 3-(2-(1-(2-((3- | 1H-NMR (400 MHz, | 613 |
| | (trifluoromethyl)pyridin- | DMSO-D6) δ 8.40 (d, J = | |
| | 2- | 4.9 Hz, 1H), 8.12 (d, J = | |
| | yl)oxy)acetyl)piperidin- | 7.6 Hz, 1H), 7.64 (s, 1H), | |
| | 4- | 7.38 (t, J = 7.7 Hz, 1H), | |
| | yl)thiazol-4-yl)-1,5- | 7.28-7.32 (m, 2H), 7.17 | |
| | dihydrobenzo[e][1, | (dd, J = 7.3, 5.4 Hz, 1H), | |
| | 3]dioxepin-6-yl | 6.08 (s, 1H), 5.28 (s, | |
| | methanesulfonate | 2H), 5.17 (d, J = 15.2 Hz, | |
| | | 1H), 4.95-5.08 (m, 3H), | |
| | | 4.33 (d, J = 14.2 Hz, 1H), | |
| | | 3.91 (d, J = 12.7 Hz, | |
| | | 1H), 3.50 (s, 3H), 3.36-3.40 | |
| | | (m, 1H), 3.23-3.24 | |
| | | (m, 1H), 2.79 (s, 1H), 2.09 | |
| | | (d, J = 14.9 Hz, 2H), | |
| | | 1.88-1.73 (m, 1H), | |
| | | 1.62-1.45 (m, 1H), | |
| 92 | 3-(2-(1-(2- | 1H-NMR (400 MHz, | 628 |
| | ((6-methyl-3- | DMSO-D6) δ 7.97 (d, J = | |
| | (trifluoromethyl)pyridin- | 7.8 Hz, 1H), 7.64 (s, 1H), | |
| | 2- | 7.38 (t, J = 7.8 Hz, 1H), | |
| | yl)oxy)acetyl)piperidin- | 7.30 (t, J = 7.9 Hz, 2H), | |
| | 4- | 7.01 (d, J = 7.8 Hz, 1H), | |
| | yl)thiazol-4-yl)-1,5- | 6.07 (s, 1H), 5.27 (d, | |
| | dihydrobenzo[e][1, | J = 14.4 Hz, 1H), 5.14-5.18 | |
| | 3]dioxepin-6-yl | (m, 2H), 4.95-5.07 (m, 3H), | |
| | methanesulfonate | 4.35 (d, J = 13.9 Hz, | |
| | | 1H), 3.94 (d, J = 13.4 Hz, | |
| | | 1H), 3.50 (s, 3H), 3.37 | |
| | | (td, J = 7.7, 3.9 Hz, 1H), | |
| | | 3.22-3.29 (m, 1H), 2.80 | |
| | | (t, J = 11.4 Hz, 1H), 2.37 | |
| | | (d, J = 29.1 Hz, 3H), | |
| | | 2.05-2.13 (m, 2H), 1.80- | |
| | | 1.86 (m, 1H), 1.52-1.57 | |
| | | (m, 1H) | |

### BIOLOGY EXAMPLES

Phytophthora infestans (Late Blight of Potato & Tomato):

IN VITRO TEST: Compounds were dissolved in 0.3% DMSO & then added to Rye Agar medium just prior to dispensing it into petri dishes. 5 mL medium with compound in the desired concentration was dispensed into 60 mm sterile petri-plates. After solidification each plate was seeded with 5 mm size mycelial disc taken form periphery of actively growing virulent culture plate. Plates were incubated in growth chambers at 18° C. temperature and 95% relative humidity for seven days and radial growth was measured. Compounds

| | | | | | | | | | | | |
|---|---|---|---|---|---|---|---|---|---|---|---|
| | | 1 | 2 | 3 | 4 | 5 | 9 | 10 | 15 | 19 | 20 |
| 27 | 28 | 29 | 30 | 31 | 32 | 33 | 34 | 35 | 36 | 37 | 38 |
| 39 | 40 | 41 | 42 | 43 | 46 | 47 | 48 | 49 | 51 | 52 | 53 |
| 54 | 59 | 60 | 61 | 62 | 63 | 64 | 65 | 66 | 67 | 69 | 70 |
| 71 | 72 | 76 | 77 | 78 | 79 | 82 | 83 | 84 | 85 | 86 | 87 |
| 88 | 89 | 90 | 91 | 92 | | | | | | | |
| | | | | at | | | | | | | |
| | | | | 30 | | | | | | | |
| | | | | ppm | | | | | | | |

gave 70% control in these tests when compared to the untreated check which showed extensive disease development.

### Greenhouse:

Compounds were dissolved in 2% DMSO/Acetone & then mixed with water to calibrated spray volume of 50 mL. This 50 mL spray solution was poured into the spray bottles for further applications. To test the preventive activity of compounds, healthy young Tomato plants raised in the greenhouse were sprayed with active compound preparation at the stated application rates inside the spray cabinets using hallow-cone nozzles. One day after treatment, the plants were inoculated with sporangial suspension (Cold sterile water) containing 0.24×10⁶ Phytophthora infestans inoculum. After inoculation the plants were kept in darkness at 15° C. during 24 hours, and then they were kept in greenhouse chamber at 18° C. temperature and 95-100% relative humidity for disease expression. A visual assessment of compound's performance was carried by rating the disease severity (0-100% scale) on treated plants on 3, 7, 10 and 15 days after application. Efficacy (% control) of the compounds was calculated by comparing the disease rating in the treatment with untreated control. The sprayed plants were also assessed for compound's phytotoxic effects by recording symptoms like necrosis, chlorosis and stunting. Compounds

| | | | | | | | | | | | | |
|---|---|---|---|---|---|---|---|---|---|---|---|---|
| 10 | 15 | 27 | 28 | 29 | 30 | 31 | 32 | 33 | 34 | 35 | 36 | 37 |
| | 38 | 39 | 40 | 41 | 42 | 52 | 61 | 62 | 66 | 67 | 76 | 77 |
| | 79 | 86 | 87 | | | | | | | | | |
| | | | at | | | | | | | | | |
| | | | 50 | | | | | | | | | |
| | | | ppm | | | | | | | | | |

gave 90% control in these tests when compared to the untreated check which showed extensive disease development. None of the compounds showed any negative crop response to any of the compounds tested.

### Example B: Plasmopara viticola Test in Grape

Compounds were dissolved in 2% DMSO/Acetone & then mixed with water to calibrated spray volume of 50 ml. This 50 ml spray solution was poured into the spray bottles for further applications.

To test the preventive activity of compounds, five week old healthy grape seedlings raised in the greenhouse were sprayed with active compound preparation at the stated application rates inside the spray cabinets using hallow-cone nozzles. One day after treatment, the plants were inoculated with inoculum suspension (Cold sterile water) containing 6×10⁶ Plasmopara viticola inoculum. The inoculated plants were then kept in greenhouse chamber at 18-21° C. temperature and 95-100% relative humidity for disease expression. Compounds

| | | | | | | | | | | | |
|---|---|---|---|---|---|---|---|---|---|---|---|
| | | | 10 | 15 | 27 | 29 | 30 | 31 | 32 | 33 | 34 |
| 35 | 36 | 37 | 38 | 39 | 41 | 42 | 52 | 61 | 62 | 66 | 76 |
| 77 | 79 | 86 | 87 | | | | | | | | |
| | | | at | | | | | | | | |
| | | | 50 | | | | | | | | |
| | | | ppm | | | | | | | | |

gave 90% control in these tests when compared to the untreated check which showed extensive disease development. None of the compounds showed any negative crop response to any of the compounds tested.

A visual assessment of compound's performance was carried by rating the disease severity (0-100% scale) on treated plants on 3, 7, 10 and 15 days after application. Efficacy (% control) of the compounds was calculated by comparing the disease rating in the treatment with untreated control. The sprayed plants were also assessed for compound's phytotoxic effects by recording symptoms like necrosis, chlorosis and stunting.

1. A compound selected from Formula I, 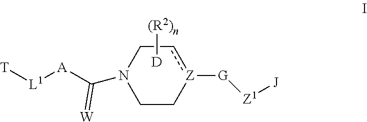
wherein,
T is selected from 5- or 6-membered aryl ring or 5- or 6-membered saturated or partially saturated cyclic ring or 5- or 6-membered heteroaryl ring or 5- or 6-membered saturated or partially saturated heterocyclic ring, wherein each ring member of heteroaryl ring is selected from C, N, O and S, and wherein each ring member of heterocyclic ring is selected from C, N, O, S(O)ₐ, C═O, C═S, S═NR⁶ and S(O)—NR⁶, and T is optionally substituted by one or more R^{1b} on carbon ring members and one or more R^{1b} on heteroatom ring members;
L¹ is O, S, NR²³,
wherein, R²³ is selected from hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylcarbonyl, C₁-C₆ haloalkylcarbonyl, C₁-C₆ alkoxycarbonyl, C₁-C₆ alkoxycarbonyl C₁-C₆ alkyl, C₁-C₆ alkylaminocarbonyl, C₁-C₆ dialkylaminocarbonyl, C₁-C₆ alkylsulfonyl and C₁-C₆ haloalkylsulfonyl;
A is C(R¹⁵)₂ or C(R¹⁵)₂—C(R¹⁵)₂;
wherein, R¹⁵ is independently selected from hydrogen, halogen, cyano, hydroxy, aldehyde, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylcarbonyl, C₁-C₆ haloalkylcarbonyl, C₁-C₆ alkoxycarbonyl, C₁-C₆ alkoxycarbonyl C₁-C₆ alkyl, C₁-C₆ alkylaminocarbonyl, C₁-C₆ dialkylaminocarbonyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl and C₁-C₆ haloalkylsulfonyl;
W is O or S;
Z is C or N;
the presentation “ [symbol] ” in ring D is a single bond when Z is N and is a single or double bond when Z is C;
“n” is an integer ranging from 0 to 9 with the proviso that when Z is N, “n” is an integer ranging from 0 to 8; and when the presentation “ [symbol] ” in ring D is a double bond then “n” is an integer ranging from 0 to 7;
G is an optionally substituted 5- or 6-membered heteroaryl ring or 5- or 6-membered saturated or partially saturated heterocyclic ring, each ring member of the heteroaryl ring is selected from C, N, O and S; and each ring member of the heterocyclic ring is selected from C, N, O, S(O)ₐ, C(═O), C(═S), S(═NR⁶) and S(O)═NR⁶; wherein, carbon ring members are substituted with one or more R^{3a} and heteroatom ring members are substituted with one or more R^{11a};
wherein,
R^{3a} is hydrogen or R^{3b}; R^{3b} is C₁-C₃ alkyl, C₁-C₃ haloalkyl, halogen, a phenyl or 5- or 6-membered heteroaromatic ring, wherein the phenyl or the 5- or 6-membered heteroaromatic ring is optionally substituted with one or more substituent independently selected from R^{4a} on carbon ring members and R^{4b} on nitrogen ring members,
R^{4a} is independently selected from C₁-C₆ alkyl, C₂-C₆ alkenyl, C₂-C₆ alkynyl, C₃-C₆ cycloalkyl, C₃-C₆ cycloalkyl C₁-C₆ alkyl, C₁-C₆ alkyl C₃-C₆ cycloalkyl, C₁-C₆ haloalkyl, C₂-C₆ haloalkenyl, C₂-C₆ haloalkynyl, C₃-C₆ halocycloalkyl, halogen, hydroxy, amino, cyano, nitro, C₁-C₄ alkoxy, C₁-C₄ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₁-C₆ dialkylamino, C₃-C₆ cycloalkylamino, C₁-C₆ alkoxy C₁-C₆ alkyl, C₁-C₆ hydroxyalkyl, C₁-C₆ alkylcarbonyl, C₁-C₆ alkoxycarbonyl, C₁-C₆ alkylcarbonyloxy, C₁-C₆ alkylcarbonylthio, C₁-C₆ alkylaminocarbonyl, C₁-C₆ dialkylaminocarbonyl and C₁-C₆ trialkylsilyl,
R^{4b} is independently selected from C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₂-C₆ haloalkynyl, C₃-C₆ halocycloalkyl and C₁-C₆ alkoxy C₁-C₆ alkyl,
R^{11a} is hydrogen or R^{11b}, wherein the R^{11b} is independently selected from C₁-C₃ alkyl, C₃-C₆ cycloalkyl, C₁-C₆ haloalkyl, C₃-C₆ halocycloalkyl;
J is a 5-, 6- or 7-membered carbocylic or heterocyclic ring, a 8- to 11-membered carbocylic or heterocyclic bicyclic ring system or a 7- to 11-membered carbocyclic or heterocyclic spirocyclic ring system, each ring member of the heterocyclic ring or ring system is selected from C, N, O, S(O)ₐ, C(═O), C(═S), and each ring or ring system is optionally substituted with one or more substituents independently selected from R⁵, or
J is selected from 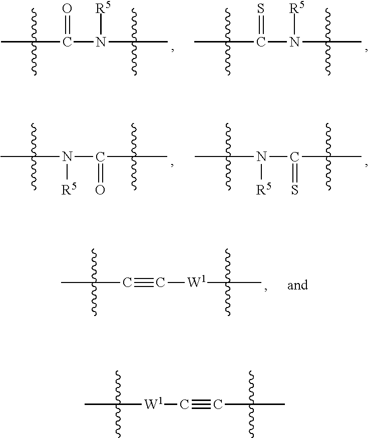
wherein W¹ is C(R⁵)₂ or CO or O or S or SO or SO₂ or NR⁵,
R⁵ is independently selected from hydrogen, halogen, cyano, hydroxy, nitro, aldehyde, carboxylic acid, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₁-C₆ alkyl C₃-C₆ cycloalkyl, C₃-C₆ cycloalkyl C₁-C₆ alkyl, C₃-C₆ cycloalkyl C₃-C₆ cycloalkyl, C₃-C₆ halocyclo C₁-C₆ alkyl, C₃-C₆ cycloalkenyl, C₃-C₆ halocycloalkenyl, C₁-C₆ alkoxy C₁-C₆ alkyl, C₃-C₆ cycloalkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylamino C₁-C₆ alkyl, C₁-C₆ dialkylamino C₁-C₆ alkyl, C₁-C₆ haloalkylamino C₁-C₆ alkyl, C₁-C₆ cycloalkylamino C₁-C₆ alkyl, C₁-C₆ alkylcarbonyl, C₁-C₆ haloalkylcarbonyl, C₃-C₆ cycloalkylcarbonyl, C₁-C₆ alkoxycarbonyl, C₃-C₆ cycloalkoxycarbonyl, C₃-C₆ cycloalkyl C₁-C₆ alkoxycarbonyl, C₁-C₆ alkylaminocarbonyl, C₁-C₆ dialkylaminocarbonyl, C₃-C₆ cycloalkylaminocarbonyl, C₁-C₆ haloalkoxy C₁-C₆ alkyl, C₁-C₆ hydroxyalkyl, C₁-C₆ alkoxy, C₁-C₆ haloalkoxy, C₃-C₆ cycloalkoxy, C₃-C₆ halocycloalkoxy, C₃-C₆ cycloalkyl C₁-C₆ alkoxy, C₂-C₆ alkenyloxy, C₂-C₆ haloalkenyloxy, C₂-C₆ alkynyloxy, C₂-C₆ haloalkynyloxy, C₁-C₆ alkoxy C₁-C₆ alkoxy, C₁-C₆ alkylcarbonyloxy, C₁-C₆ haloalkylcarbonyloxy, C₃-C₆ cycloalkylcarbonyloxy, C₁-C₆ alkylcarbonyl C₁-C₆ alkoxy, C₁-C₆ alkylthio, C₁-C₆ haloalkylthio, C₃-C₆ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₆ cycloalkylsulfonyl, C₁-C₆ trialkylsilyl, C₁-C₆ alkylsulfonylamino, C₁-C₆ haloalkylsulfonylamino or —Z²Q,
wherein, Z¹ and Z² are independently a direct bond, O, C═O, C═S, S(O)ₐ, CHR²⁰ or NR²¹;
wherein, R²⁰ is independently selected from hydrogen, C₁-C₄ alkyl or C₁-C₄ haloalkyl; and R²¹ is independently selected from hydrogen, C₁-C₈ alkyl, C₁-C₈ haloalkyl, C₃-C₈ cycloalkyl, C₁-C₆ alkylcarbonyl, C₁-C₈ haloalkylcarbonyl, C₁-C₈ alkoxycarbonyl or C₁-C₈ haloalkoxycarbonyl.
Q is independently selected from phenyl, benzyl, naphthyl, a 5- or 6-membered aryl ring, an 8- to 11-membered aryl multi-cyclic ring system, an 8- to 11-membered aryl fused ring system, a 5- or 6-membered heteroaryl ring, an 8- to 11-membered heteroaryl multi-cyclic ring system or an 8- to 11-membered heteroaryl fused ring system, each ring member of the ring or the ring system is selected from C, N, O and S, and each ring or ring system is optionally substituted with one or more substituents independently selected from R⁷ on carbon atom ring members and R¹² on hetero atom ring members, or
Q is independently selected from a 3- to 7-membered nonaromatic carbocyclic ring, a 5-, 6- or 7-membered nonaromatic heterocyclic ring, an 8- to 15-membered nonaromatic multi-cyclic ring system or an 8- to 15-membered nonaromatic fused ring system, each ring member of the ring or the ring system is selected from C, N, O, S(O)ₐ, C(═O), C(═S), S(═NR⁶) and S(═O)═NR⁶ & SiR¹⁶R¹⁷, and each ring or ring system is optionally substituted with one or more substituents independently selected from R⁷ on carbon atom ring members and R¹² on hetero atom ring members; or
J & Q together form a fragment selected from M1 and M2: 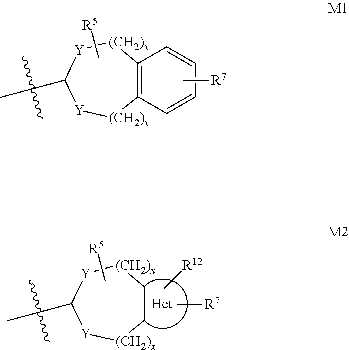
wherein, x in the fragments M1 and M2 is an integer ranging from 0 to 2 and Y is selected from N, O and S,
wherein, R^{1a}, R^{1b}, R⁷ and R¹² are independently selected from hydrogen, halogen, hydroxy, cyano, nitro, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₁-C₆ alkyl C₃-C₈ cycloalkyl, C₃-C₈ cycloalkyl C₁-C₆ alkyl, C₃-C₈ cycloalkyl C₃-C₈ cycloalkyl, C₃-C₈halocycloalkyl C₁-C₆ alkyl, C₁-C₆ alkoxy C₁-C₆ alkyl, C₃-C₈ cycloalkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylamino, C₁-C₆ dialkylamino, C₁-C₆ alkylamino C₁-C₆ alkyl, C₁-C₆ dialkylamino C₁-C₆ alkyl, C₁-C₆ haloalkylamino C₁-C₆ alkyl, C₃-C₈ cycloalkylamino, C₃-C₈ cycloalkylamino C₁-C₆ alkyl, C₁-C₆ alkylcarbonyl, C₁-C₆ haloalkylcarbonyl, C₃-C₈ cycloalkylcarbonyl, C₁-C₆ alkoxycarbonyl, C₃-C₈ cycloalkoxycarbonyl, C₁-C₆ alkylaminocarbonyl, C₁-C₆ dialkylaminocarbonyl, C₃-C₈ cycloalkylaminocarbonyl, C₁-C₆ haloalkoxy C₁-C₆ alkyl, C₁-C₆ hydroxyalkyl, C₁-C₆ hydroxyalkenyl, C₁-C₆ hydroxyalkynyl, C₁-C₆ alkoxy, C₁-C₆ haloalkoxy, C₁-C₆ cycloalkoxy, C₃-C₈ halocycloalkoxy, C₃-C₈ cycloalkyl C₁-C₆ alkoxy, C₂-C₆ alkenyloxy, C₂-C₆ haloalkenyloxy, C₂-C₆ alkynyloxy, C₂-C₆ haloalkynyloxy, C₁-C₆ alkoxy C₁-C₆ alkoxy, C₁-C₆ alkylcarbonyloxy, C₁-C₆ haloalkylcarbonyloxy, C₃-C₆ cycloalkylcarbonyloxy, C₁-C₆ alkylcarbonyl C₁-C₆ alkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ cycloalkylsulfinyl, C₁-C₆ trialkylsilyl, C₁-C₆ alkylsulfonylamino, C₁-C₆ haloalkylsulfonylamino, C₁-C₆ alkylcarbonylthio, C₁-C₆ alkylsulfonyloxy, C₁-C₆ alkylsulfinyloxy, arylsulfonyloxy, arylsulfinyloxy, arylsulfonyl, arylsulfinyl, C₁-C₆ cyanoalkyl, C₂-C₆ alkenylcarbonyloxy, C₁-C₆ alkoxy C₁-C₆ alkylthio, C₁-C₆ alkylthio C₁-C₆ alkoxy, C₂-C₆ haloalkenylcarbonyloxy, C₁-C₆ alkoxy C₂-C₆ alkynyl, C₂-C₆ alkynylthio, C₃-C₈ halocycloalkylcarbonyloxy, C₂-C₆ alkenylamino, C₂-C₆ alkynylamino, C₁-C₆ haloalkylamino, C₃-C₈ cycloalkyl C₁-C₆ alkylamino, C₁-C₆ alkoxyamino, C₁-C₆ haloalkoxyamino, C₁-C₆ alkylcarbonylamino, C₁-C₆ haloalkylcarbonylamino, C₁-C₆ alkoxycarbonylamino, C₂-C₆ alkenylthio, C₁-C₆ haloalkoxycarbonyl, C₁-C₆ alkoxy C₁-C₆ alkylcarbonyl, C₁-C₆ haloalkoxycarbonylamino, C₁-C₆ alkoxy C₁-C₆ alkylaminocarbonyl, C₁-C₆ alkylthiocarbonyl, C₃-C₈ cycloalkenyloxy C₁-C₆ alkyl, C₁-C₆ alkoxy C₁-C₆ alkoxycarbonyl, C₁-C₆ haloalkoxy C₁-C₆ haloalkoxy, C₁-C₆ alkoxy C₁-C₆ haloalkoxy, C₃-C₈ halocycloalkoxy C₁-C₆ alkyl, C₁-C₆ dialkylaminocarbonylamino, C₁-C₆ alkoxy C₂-C₆ alkenyl, C₁-C₆ alkylthiocarbonyloxy, C₁-C₆ haloalkoxy C₁-C₆ alkoxy, C₁-C₆ haloalkylsulfonyloxy, C₁-C₆ alkoxy C₁-C₆ haloalkyl, C₁-C₆ dihaloalkylamino, C₁-C₆ dialkoxy C₁-C₆ alkyl, C₁-C₆ alkylaminocarbonylamino, C₁-C₆ haloalkoxy C₁-C₆ haloalkyl, C₁-C₆ alkylaminocarbonyl C₁-C₆ alkylamino, C₁-C₆ trialkylsilyl C₂-C₆ alkynyloxy, C₁-C₆ trialkylsilyloxy, C₁-C₆ trialkylsilyl C₂-C₆ alkynyl, C₁-C₆ cyanoalkoxy C₁-C₆ alkyl, C₁-C₆ dialkylthio C₁-C₆ alkyl, C₁-C₆ alkoxysulfonyl, C₃-C₈ halocycloalkoxycarbonyl, C₁-C₆ alkylcy C₃-C₈ cloalkylcarbonyl, C₃-C₃ halocyclo C₁-C₆ alkylcarbonyl, C₂-C₆ alkenyloxycarbonyl, C₂-C₆ alkynyloxycarbonyl, C₁-C₆ cyanoalkoxycarbonyl, C₁-C₆ alkylthio C₁-C₆ alkoxycarbonyl, C₂-C₆ alkynylcarbonyloxy, C₂-C₆ haloalkynylcarbonyloxy, cyanocarbonyloxy, C₁-C₆ cyanoalkylcarbonyloxy, C₃-C₈ cycloalkylsulphonyloxy, C₃-C₈ cycloalkyl C₁-C₆ alkylsulphonyloxy, C₃-C₈ halocycloalkylsulphonyloxy, C₂-C₆ alkenylsulphonyloxy, C₂-C₆ alkynylsulphonyloxy, C₁-C₆ cyanoalkylsulphonyloxy, C₂-C₆ haloalkenylsulphonyloxy, C₂-C₅ haloalkynylsulphonyloxy, C₂-C₆ alkynylcycloalkyloxy, C₂-C₆ cyanoalkenyloxy, C₂-C₆ cyanoalkynyloxy, C₁-C₆ alkoxycarbonyloxy, C₂-C₆ alkenyloxycarbonyloxy, C₂-C₆ alkynyloxycarbonyloxy, C₁-C₆ alkoxyalkylcarbonyloxy, sulfilimines, sulfoximines, SF₅ or Z²Q,
R¹⁶ and R¹⁷ are independently selected from C₁-C₆ alkyl, C₂-C₆ alkenyl, C₂-C₆ alkynyl, C₃-C₆ cycloalkyl, C₃-C₆ halocycloalkyl, C₁-C₆ cycloalkyl C₁-C₆ alkyl, C₁-C₆ alkyl C₃-C₆ cycloalkyl, C₁-C₆ haloalkyl, C₁-C₆ alkoxy and C₁-C₆ haloalkoxy,
R⁵ and R⁷ or R⁵ and R¹² taken together with the atoms linking R⁵ and R⁷ or R¹² to form a saturated, unsaturated or partially unsaturated 4- to 7-membered ring, each ring members selected from C, N, O, S(O)ₐ, C═O, C═S, S═NR⁶ and S(O)═NR⁶, and said ring optionally substituted on ring members other than the atoms linking R⁵ and R¹⁵ or R¹² with R⁸, wherein, R⁸ is selected from halogen, C₁-C₆ alkyl, C₁-C₆ haloalkyl, C₃-C₈ cycloalkyl, and C₃-C₈ cycloalkyl;
R⁵ and R⁶ are independently selected from hydrogen, halogen, cyano, hydroxy, aldehyde, carboxylic acid, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₁-C₆ alkyl C₃-C₆ cycloalkyl, C₃-C₆ cycloalkyl C₁-C₆ alkyl, C₃-C₆ halocycloalkyl C₁-C₆ alkyl, C₃-C₆ cycloalkenyl, C₃-C₆ halocycloalkenyl, C₁-C₆ alkoxy C₁-C₆ alkyl, C₁-C₆ alkylthio C₁-C₆ alkyl, C₁-C₆ alkylsulfinyl C₁-C₆ alkyl, C₁-C₆ alkylsulfonyl C₁-C₆ alkyl, C₁-C₆ alkylamino C₁-C₆ alkyl, C₁-C₆ dialkylamino C₁-C₆ alkyl, C₁-C₆ haloalkylamino C₁-C₆ alkyl, C₁-C₆ alkylcarbonyl, C₁-C₆ haloalkylcarbonyl, C₃-C₆ cycloalkylcarbonyl, C₁-C₆ alkoxycarbonyl, C₃-C₆ cycloalkoxycarbonyl, C₃-C₆ cycloalkyl C₁-C₆ alkoxycarbonyl, C₁-C₆ alkylaminocarbonyl, C₁-C₆ dialkylaminocarbonyl, C₁-C₆ alkoxy, C₁-C₆ haloalkoxy, C₃-C₆ cycloalkoxy, C₃-C₆ halocycloalkoxy, C₂-C₆ alkenyloxy, C₂-C₆ haloalkenyloxy, C₂-C₆ alkynyloxy, C₂-C₆ haloalkynyloxy, C₁-C₆ alkoxy C₁-C₆ alkoxy, C₁-C₆ alkylcarbonyloxy, C₁-C₆ haloalkylcarbonyloxy, C₁-C₆ alkylthio, C₁-C₆ haloalkylthio, C₃-C₆ cycloalkylthio, C₁-C₆ alkylamino, C₁-C₆ dialkylamino, C₁-C₆ haloalkylamino, C₁-C₆ halodialkylamino, C₃-C₆ cycloalkylamino, C₁-C₆ alkylcarbonylamino, C₁-C₆ haloalkylcarbonylamino, C₁-C₆ alkylsulfonylamino and C₁-C₆ haloalkylsulfonylamino, or
two R² are taken together as C₁-C₄ alkylene or C₂-C₄ alkenylene or —CH═CH—CH═CH— to form a bridged bicyclic or fused bicyclic ring system optionally substituted with a substituent selected from C₁-C₆ alkyl, C₁-C₆ haloalkyl, C₁-C₆ alkoxy, C₁-C₆ haloalkoxy, halogen, hydroxy, amino, cyano and nitro,
proviso that the compounds are excluded from the definition of Formula I:
Ethanone, 1-[4-[4-(5-methyl-3-phenyl-4-isoxazolyl)-2-thiazolyl]-1-piperidinyl]-2-[[5-(trifluoromethyl)-2-pyridinyl]thio]-(CAS RN-1023141-80-1);
Benzamide, 2-[[2-[4-[4-[3-(3,4-dichlorophenyl)-5-isoxazolyl]-2-thiazolyl]-1-piperidinyl]-2-oxoethyl]thio]-4-ethoxy-(CAS RN-1177816-84-0);
Ethanone, 2-[(2-chloro-4-fluorophenyl)thio]-1-[4-[4-[3-(3,4-dichlorophenyl)-5-isoxazolyl]-2-thiazolyl]-1-piperidinyl]-(CAS RN-1177683-42-9);
Ethanone, 2-(cyclohexyloxy)-1-[4-[4-[5-(2,6-difluorophenyl)-4,5-dihydro-3-isoxazolyl]-2-thiazolyl]-1-piperidinyl]-(CAS RN-1173972-38-7);
1-Propanone, 2-(4-chlorophenoxy)-2-methyl-1-[4-[4-(5-methyl-3-phenyl-4-isoxazolyl)-2-thiazolyl]-1-piperidinyl]-(CAS RN-1136418-28-4);
Ethanone, 2-[(2-chloro-4-fluorophenyl)thio]-1-[4-[4-(5-methyl-3-phenyl-4-isoxazolyl)-2-thiazolyl]-1-piperidinyl]-(CAS RN-1023177-70-9);
Benzenesulfonamide, N-methyl-2-[[2-[4-[4-(5-methyl-3-phenyl-4-isoxazolyl)-2-thiazolyl]-1-piperidinyl]-2-oxoethyl]thio]-(CAS RN-1023156-55-9);
Benzenesulfonamide, 2-[[2-[4-[4-[3-(3,4-dichlorophenyl)-5-isoxazolyl]-2-thiazolyl]-1-piperidinyl]-2-oxoethyl]thio]-N-methyl-(CAS RN-1022602-51-2);
Ethanone, 1-[4-[4-(5-methyl-3-phenyl-4-isoxazolyl)-2-thiazolyl]-1-piperidinyl]-2-(2,3,4,5,6-pentafluorophenoxy)-(CAS RN-1022567-65-2);
Ethanone, 1-[4-[4-[3-(3,4-dichlorophenyl)-5-isoxazolyl]-2-thiazolyl]-1-piperidinyl]-2-[(4-methyl phenyl)sulfonyl]-(CAS RN-1022566-90-0);
Ethanone, 2-(2,4-dichlorophenoxy)-1-[4-[4-[3-(3,4-dichlorophenyl)-5-isoxazolyl]-2-thiazolyl]-1-piperidinyl]-(CAS RN-1022328-76-2);
Ethanone, 2-(2,4-dichlorophenoxy)-1-[4-[4-(5-methyl-3-phenyl-4-isoxazolyl)-2-thiazolyl]-1-piperidinyl]-(CAS RN-1022068-84-3);
Ethanone, 1-[4-[4-[3-(3,4-dichlorophenyl)-5-isoxazolyl]-2-thiazolyl]-1-piperidinyl]-2-(2,3,4,5,6-pentafluorophenoxy)-(CAS RN-1022028-25-6);
1-Propanone, 1-[4-[4-(5-methyl-3-phenyl-4-isoxazolyl)-2-thiazolyl]-1-piperidinyl]-3-[(2-methylphenyl)thio]-(CAS RN-1022326-33-5); and
1-Propanone, 1-[4-[4-[3-(3,4-dichlorophenyl)-5-isoxazolyl]-2-thiazolyl]-1-piperidinyl]-3-[(2-methylphenyl)thio]-(CAS RN 1024410-18-1),
the salts, isomers, metal complexes, N-oxides and polymorphs thereof.

2. The compound as claimed in claim 1, wherein,
L¹ is O, S or NH;
A is C(R¹⁵)₂;
W is O;
Z is C;
the presentation “ [symbol] ” in ring D is a single bond;
“n” is an integer ranging from 0 to 9;
G is an optionally substituted 5-membered heteroaryl;
J is a 5-membered heterocyclic ring, wherein heteroatom ring members are selected from N and O;
R⁵ is Z²Q;
Z¹ and Z² are a direct bond; and
Q is phenyl or 6-membered heteroaryl ring; or
J and Q together form a fragment selected from M1′ or M2′ 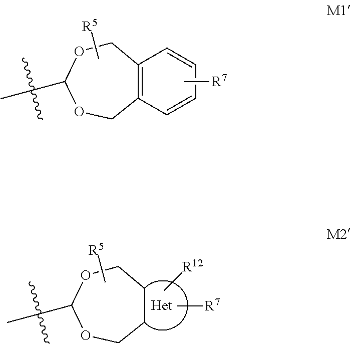
R⁵ and R⁷ each has the same meaning as defined in the claim 1.

3. The compound as claimed in claim 1, wherein,
T is T1 to T47;
G is G1 to G63;
J is J1 to J82; and
Q is Q1 to Q99.

4. The compound as claimed in claim 1, wherein,
T is selected from T11, T25, T26, T37, T38, and T39;
G is selected from G1, G15, G37, G45, G61;
J is selected from J30, J11 and J29; and
Q is Q45, Q32, Q33, Q34, Q36, Q38 and Q39.

5. The compound as claimed in claim 1, comprising
1-(4-(4-(5-(2,6-difluorophenyl)-4,5-dihydroisoxazol-3-yl)thiazol-2-yl)piperidin-1-yl)-2-((1-methyl-1H-pyrazol-3-yl)oxy)ethan-1-one; 1-(4-(4-(5-(2,6-dichlorophenyl)-4,5-dihydroisoxazol-3-yl)thiazol-2-yl)piperidin-1-yl)-2-((1-methyl-3-(trifluoromethyl)-1H-pyrazol-5-yl)oxy)ethan-1-one; 1-(4-(4-(5-(2,6-difluorophenyl)-4,5-dihydroisoxazol-3-yl)thiazol-2-yl)piperidin-1-yl)-2-((1-methyl-3-(trifluoromethyl)-1H-pyrazol-5-yl)oxy)ethan-1-one; 3-chloro-2-(3-(2-(1-(2-((1-methyl-3-(trifluoromethyl)-1H-pyrazol-5-yl)oxy)acetyl)piperidin-4-yl)thiazol-4-yl)-4,5-dihydroisoxazol-5-yl)phenylmethanesulfonate; 3-chloro-2-(3-(2-(1-(2-((1-methyl-1H-pyrazol-3-yl)oxy)acetyl)piperidin-4-yl)thiazol-4-yl)-4,5-dihydroisoxazol-5-yl)phenylmethanesulfonate; 1-(4-(4-(5-(2,6-dichlorophenyl)-4,5-dihydroisoxazol-3-yl)thiazol-2-yl)piperidin-1-yl)-2-((5-(difluoromethyl)-1-methyl-1H-pyrazol-3-yl)oxy)ethan-1-one; 2-((5-(difluoromethyl)-1-methyl-1H-pyrazol-3-yl)oxy)-1-(4-(4-(5-(2,4,6-trichlorophenyl)-4,5-dihydroisoxazol-3-yl)thiazol-2-yl)piperidin-1-yl)ethan-1-one; 3-chloro-2-(3-(2-(1-(2-((5-(difluoromethyl)-1-methyl-1H-pyrazol-3-yl)oxy)acetyl)piperidin-4-yl)thiazol-4-yl)-4,5-dihydroisoxazol-5-yl)phenylmethanesulfonate; 2-((5-(difluoromethyl)-1-methyl-1H-pyrazol-3-yl)oxy)-1-(4-(4-(5-(2,6-di fluorophenyl)-4,5-dihydroisoxazol-3-yl)thiazol-2-yl)piperidin-1-yl)ethan-1-one; 2-((1-methyl-3-(trifluoromethyl)-1H-pyrazol-5-yl)oxy)-1-(4-(4-(5-(2,4,6-trichlorophenyl)-4, 5-dihydroisoxazol-3-yl)thiazol-2-yl)piperidin-1-yl)ethan-1-one; 2-((4-bromo-1-methyl-5-(trifluoromethyl)-1H-pyrazol-3-yl)oxy)-1-(4-(4-(5-(2,6-dichlorophenyl)-4,5-dihydroisoxazol-3-yl)thiazol-2-yl)piperidin-1-yl)ethan-1-one; 2-(3-(2-(1-(2-((4-bromo-1-methyl-5-(trifluoromethyl)-1H-pyrazol-3-yl)oxy)acetyl)piperidin-4-yl)thiazol-4-yl)-4,5-dihydroisoxazol-5-yl)-3-chlorophenylmethanesulfonate; 2-((4-bromo-1-methyl-5-(trifluoromethyl)-1H-pyrazol-3-yl)oxy)-1-(4-(4-(5-(2,4,6-trichlorophenyl)-4,5-dihydroisoxazol-3-yl)thiazol-2-yl)piperidin-1-yl)ethan-1-one; 2-((4-bromo-1-methyl-5-(trifluoromethyl)-1H-pyrazol-3-yl)oxy)-1-(4-(4-(5-(2,6-di fluorophenyl)-4,5-dihydroisoxazol-3-yl)thiazol-2-yl)piperidin-1-yl)ethan-1-one; 1-(4-(4-(5-(2,6-dichlorophenyl)-4,5-dihydroisoxazol-3-yl)thiazol-2-yl)piperidin-1-yl)-2-((1-methyl-5-(trifluoromethyl)-1H-pyrazol-3-yl)oxy)ethan-1-one; 2-((4-bromo-1-methyl-3-(trifluoromethyl)-1H-pyrazol-5-yl)oxy)-1-(4-(4-(5-(2,6-dichlorophenyl)-4,5-dihydroisoxazol-3-yl)thiazol-2-yl)piperidin-1-yl)ethan-1-one; 2-(3-(2-(1-(2-((4-bromo-1-methyl-3-(trifluoromethyl)-1H-pyrazol-5-yl)oxy)acetyl)piperidin-4-yl)thiazol-4-yl)-4,5-dihydroisoxazol-5-yl)-3-chlorophenylmethanesulfonate; 2-((4-bromo-1-methyl-3-(trifluoromethyl)-1H-pyrazol-5-yl)oxy)-1-(4-(4-(5-(2,6-difluorophenyl)-4,5-dihydroisoxazol-3-yl)thiazol-2-yl)piperidin-1-yl)ethan-1-one; 3-chloro-2-(3-(2-(1-(2-((1-methyl-5-(trifluoromethyl)-1H-pyrazol-3-yl)oxy)acetyl)piperidin-4-yl)thiazol-4-yl)-4,5-dihydroisoxazol-5-yl)phenylmethanesulfonate; 1-(4-(4-(5-(2,6-difluorophenyl)-4,5-dihydroisoxazol-3-yl)thiazol-2-yl)piperidin-1-yl)-2-((1-methyl-5-(trifluoromethyl)-1H-pyrazol-3-yl)oxy)ethan-1-one; 2-((1-methyl-5-(trifluoromethyl)-11H-pyrazol-3-yl)oxy)-1-(4-(4-(5-(2,4,6-trichlorophenyl)-4,5-dihydroisoxazol-3-yl)thiazol-2-yl)piperidin-1-yl)ethan-1-one; 2-((4-bromo-1-methyl-3-(trifluoromethyl)-1H-pyrazol-5-yl)oxy)-1-(4-(4-(5-(2,4,6-trichlorophenyl)-4,5-dihydroisoxazol-3-yl)thiazol-2-yl)piperidin-1-yl)ethan-1-one; 2-(2,4-dichlorophenoxy)-1-(4-(4-(5-(2,6-dichlorophenyl)-4,5-dihydroisoxazol-3-yl)thiazol-2-yl)piperidin-1-yl)propan-1-one; 3-chloro-2-(3-(2-(1-(2-(2,4-dichlorophenoxy)propanoyl)piperidin-4-yl)thiazol-4-yl)-4,5-dihydroisoxazol-5-yl)phenylmethanesulfonate; 2-(2,4-dichlorophenoxy)-1-(4-(4-(5-(2,4,6-trichlorophenyl)-4,5-dihydroisoxazol-3-yl)thiazol-2-yl)piperidin-1-yl)propan-1-one; 2-(2,4-dichlorophenoxy)-1-(4-(4-(5-(2,6-difluorophenyl)-4,5-dihydroisoxazol-3-yl)thiazol-2-yl)piperidin-1-yl)propan-1-one; 1-(4-(4-(5-(2,6-dichlorophenyl)-4,5-dihydroisoxazol-3-yl)thiazol-2-yl)piperidin-1-yl)-2-((3-(trifluoromethyl)pyridin-2-yl)oxy)ethan-1-one; 3-chloro-2-(3-(2-(1-(2-((3-(trifluoromethyl)pyridin-2-yl)oxy)acetyl)piperidin-4-yl)thiazol-4-yl)-4,5-dihydroisoxazol-5-yl)phenylmethanesulfonate; 1-(4-(4-(5-(2,6-difluorophenyl)-4,5-dihydroisoxazol-3-yl)thiazol-2-yl)piperidin-1-yl)-2-((3-(trifluoromethyl)pyridin-2-yl)oxy)ethan-1-one; 1-(4-(4-(5-(2,4,6-trichlorophenyl)-4,5-dihydroisoxazol-3-yl)thiazol-2-yl)piperidin-1-yl)-2-((3-(trifluoromethyl)pyridin-2-yl)oxy)ethan-1-one; 2-((5-chloro-3-(trifluoromethyl)pyridin-2-yl)oxy)-1-(4-(4-(5-(2,6-dichlorophenyl)-4,5-dihydroisoxazol-3-yl)thiazol-2-yl)piperidin-1-yl)ethan-1-one; 3-chloro-2-(3-(2-(1-(2-((5-chloro-3-(trifluoromethyl)pyridin-2-yl)oxy)acetyl)piperidin-4-yl)thiazol-4-yl)-4,5-dihydroisoxazol-5-yl)phenylmethanesulfonate; 2-((5-chloro-3-(trifluoromethyl)pyridin-2-yl)oxy)-1-(4-(4-(5-(2,6-difluoropbenyl)-4,5-dihydroisoxazol-3-yl)thiazol-2-yl)piperidin-1-yl)ethan-1-one; 2-(5-cloro-3-(trifluoromethyl)pyridin-2-yl)oxy)-1-(4-(4-(5-(2,4,6-trichlorophenyl)-4,5-dihydroisoxazol-3-yl)thiazol-2-yl)piperidin-1-yl)ethan-1-one; 2-((5-bromo-3-(trifluoromethyl)pyridin-2-yl)oxy)-1-(4-(4-(5-(2,6-dichlorophenyl)-4,5-dihydroisoxazol-3-yl)thiazol-2-yl)piperidin-1-yl)ethan-1-one; 2-(3-(2-(1-(2-((5-bromo-3-(trifluoromethyl)pyridin-2-yl)oxy)acetyl)piperidin-4-yl)thiazol-4-yl)-4,5-dihydroisoxazol-5-yl)-3-chlorophenylmethanesulfonate; 2-((5-bromo-3-(trifluoromethyl)pyridin-2-yl)oxy)-1-(4-(4-(5-(2,6-difluorophenyl)-4,5-dihydroisoxazol-3-yl)thiazol-2-yl)piperidin-1-yl)ethan-1-one; 2-((5-bromo-3-(trifluoromethyl)pyridin-2-yl)oxy)-1-(4-(4-(5-(2,4,6-trichlorophenyl)-4,5-dihydroisoxazol-3-yl)thiazol-2-yl)piperidin-1-yl)ethan-1-one; 2-((6-methyl-3-(trifluoromethyl)pyridin-2-yl)oxy)-1-(4-(4-(5-(2,4,6-trichlorophenyl)-4,5-dihydroisoxazol-3-yl)thiazol-2-yl)piperidin-1-yl)ethan-1-one; 1-(4-(4-(5-(2,6-difluorophenyl)-4,5-dihydroisoxazol-3-yl)thiazol-2-yl)piperidin-1-yl)-2-((6-methyl-3-(trifluoromethyl)pyridin-2-yl)oxy)ethan-1-one; 1-(4-(4-(5-(2,6-dichlorophenyl)-4,5-dihydroisoxazol-3-yl)thiazol-2-yl)piperidin-1-yl)-2-((6-methyl-3-(trifluoromethyl)pyridin-2-yl)oxy)ethan-1-one; 3-chloro-2-(3-(2-(1-(2-((6-methyl-3-(trifluoromethyl)pyridin-2-yl)oxy)acetyl)piperidin-4-yl)thiazol-4-yl)-4,5-dihydroisoxazol-5-yl)phenylmethanesulfonate; 1-(4-(4-(5-(2,6-difluorophenyl)-4,5-dihydroisoxazol-3-yl)thiazol-2-yl)piperidin-1-yl)-2-((4-(trifluoromethyl)pyridin-2-yl)oxy)ethan-1-one; 1-(4-(4-(5-(2,4,6-trichlorophenyl)-4,5-dihydroisoxazol-3-yl)thiazol-2-yl)piperidin-1-yl)-2-((4-(trifluoromethyl)pyridin-2-yl)oxy)ethan-1-one; 1-(4-(4-(5-(2,6-dichlorophenyl)-4,5-dihydroisoxazol-3-yl)thiazol-2-yl)piperidin-1-yl)-2-((4-(trifluoromethyl)pyridin-2-yl)oxy)ethan-1-one; 3-chloro-2-(3-(2-(1-(2-((4-(trifluoromethyl)pyridin-2-yl)oxy)acetyl)piperidin-4-yl)thiazol-4-yl)-4,5-dihydroisoxazol-5-yl)phenylmethanesulfonate; 1-(4-(4-(5-(2,6-dichlorophenyl)-4,5-dihydroisoxazol-3-yl)thiazol-2-yl)piperidin-1-yl)-2-((5-(trifluoromethyl)pyridin-2-yl)oxy)ethan-1-one; 3-chloro-2-(3-(2-(1-(2-((5-(trifluoromethyl)pyridin-2-yl)oxy)acetyl)piperidin-4-yl)thiazol-4-yl)-4,5-dihydroisoxazol-5-yl)phenylmethanesulfonate; 1-(4-(4-(5-(2,6-difluorophenyl)-4,5-dihydroisoxazol-3-yl)thiazol-2-yl)piperidin-1-yl)-2-((5-(trifluoromethyl)pyridin-2-yl)oxy)ethan-1-one; 1-(4-(4-(5-(2,4,6-trichlorophenyl)-4,5-dihydroisoxazol-3-yl)thiazol-2-yl)piperidin-1-yl)-2-((5-(trifluoromethyl)pyridin-2-yl)oxy)ethan-1-one; 2-((3-bromo-5-(trifluoromethyl)pyridin-2-yl)oxy)-1-(4-(4-(5-(2,6-dichlorophenyl)-4,5-dihydroisoxazol-3-yl)thiazol-2-yl)piperidin-1-yl)ethan-1-one; 2-(3-(2-(1-(2-((3-bromio-5-(trifluoromethyl)pyridin-2-yl)oxy)acetyl)piperidin-4-yl)thiazol-4-yl)-4,5-dihydroisoxazol-5-yl)-3-chlorophenylmethanesulfonate; 2-((3-bromo-5-(trifluoromethyl)pyridin-2-yl)oxy)-1-(4-(4-(5-(2,6-difluorophenyl)-1,5-dihydroisoxazol-3-yl)thiazol-2-yl)piperidin-1-yl)ethan-1-one; 2-((3-bromo-5-(trifluoromethyl)pyridin-2-yl)oxy)-1-(4-(4-(5-(2,4,6-trichlorophenyl)-4,5-dihydroisoxazol-3-yl)thiazol-2-yl)piperidin-1-yl)ethan-1-one; 1-(4-(4-(5-(2,6-dichlorophenyl)-4,5-dihydroisoxazol-3-yl)thiazol-2-yl)piperidin-1-yl)-2-((5-(trifluoromethyl)pyridin-2-yl)thio)ethan-1-one; 3-chloro-2-(3-(2-(1-(2-((5-(trifluoromethyl)pyridin-2-yl)thio)acetyl)piperidin-4-yl)thiazol-4-yl)-4,5-dihydroisoxazol-5-yl)phenylmethanesulfonate; 1-(4-(4-(5-(2,6-difluorophenyl)-4,5-dihydroisoxazol-3-yl)thiazol-2-yl)piperidin-1-yl)-2-((5-(trifluoromethyl)pyridine-2-yl)thio)ethan-1-one; 1-(4-(4-(5-(2,4,6-trichlorophenyl)-4,5-dihydroisoxazol-3-yl)thiazol-2-yl)piperidin-1-yl)-2-((5-(trifluoromethyl)pyridin-2-yl)thio)ethan-1-one; 1-(4-(4-(1,5-dihydrobenzo[e][1,3]dioxepin-3-yl)thiazol-2-yl)piperidin-1-yl)-2-((3-(trifluoromethyl)pyridin-2-yl)oxy)ethan-1-one; 3-chloro-2-(3-(2-(1-(2-((3-(trifluoromethyl)pyridin-2-yl)thio)acetyl)piperidin-4-yl)thiazol-4-yl)-4,5-dihydroisoxazol-5-yl)phenylmethanesulfonate; 3-chloro-2-(3-(2-(1-(2-((3-(trifluoromethyl)pyridin-2-yl)thio)acetyl)piperdin-4-yl)thiazol-4-yl)-4, 5-dihydroisoxazol-5-yl)phenylmethanesulfonate; 1-(4-(4-(5-(2,6-difluorophenyl)-4,5-dihydroisoxazol-3-yl)thiazol-2-yl)piperidin-1-yl)-2-((3-(trifluoromethyl)pyridin-2-yl)thio)ethan-1-one; 1-(4-(4-(5-(2,4,6-tichlorophenyl)-4,5-dihydroisoxazol-3-yl)thiazol-2-yl)piperidin-1-yl)-2-((3-(trifluoromethyl)pyridin-2-yl)thio)ethan-1-one; 1-(4-(4-(5-(3-(trifluoromethyl)phenyl)-4,5-dihydroisoxazol-3-yl)thiazol-2-yl)piperidin-1-yl)-2-((3-(trifluoromethyl)pyridin-2-yl)oxy)ethan-1-one; 2-((3-chloro-5-(trifluoromethyl)pyridin-2-yl)thio)-1-(4-(4-(5-(2,6-difluorophenyl)-4,5-dihydroisoxazol-3-yl)thiazol-2-yl)piperidin-1-yl)ethan-1-one; 1-(4-(4-(5-(2-fluorophenyl)-4,5-dihydroisoxazol-3-yl)thiazol-2-yl)piperidin-1-yl)-2-((3-(trifluoromethyl)pyridin-2-yl)oxy)ethan-1-one; 2-((5-chloro-3-(trifluoromethyl)pyridin-2-yl)oxy)-1-(4-(4-(5-(4-methoxyphenyl)-4,5-dihydroisoxazol-3-yl)thiazol-2-yl)piperidin-1-yl)ethan-1-one; 2-((3-chloro-5-(trifluoromethyl)pyridin-2-yl)thio)-1-(4-(4-(5-(2,4,6-trichlorophenyl)-4,5-dihydroisoxazol-3-yl)thiazol-2-yl)piperidin-1-yl)ethan-1-one; N-(((2-(4-(4-(5-(2,6-difluorophenyl)-4,5-dihydroisoxazol-3-yl)thiazol-2-yl)piperidin-1-yl)-2-oxoethyl)(5-(trifluoromethyl)pyridin-2-yl)amino)(dimethylamino)methylene)-N-methylmethanaminium; 1-(4-(4-(5-(2,6-dichlorophenyl)-4,5-dihydroisoxazol-3-yl)thiazol-2-yl)piperidin-1-yl)-2-((6-(trifluoromethyl)pyridin-2-yl)oxy)ethan-1-one; 3-chloro-2-(3-(2-(1-(2-((6-(trifluoromethyl)pyridin-2-yl)oxy)acetyl)piperidin-4-yl)thiazol-4-yl)-4,5-dihydroisoxazol-5-yl)phenylmethanesulfonate; 1-(4-(4-(5-(2,6-difluorophenyl)-4,5-dihydroisoxazol-3-yl)thiazol-2-yl)piperidin-1-yl)-2-((6-(trifluoromethyl)pyridin-2-yl)oxy)ethan-1-one; 1-(4-(4-(5-(2,4,6-trichlorophenyl)-4,5-dihydroisoxazol-3-yl)thiazol-2-yl)piperidin-1-yl)-2-((6-(trifluoromethyl)pyridin-2-yl)oxy)ethan-1-one; 2-((3-chloro-5-(trifluoromethyl)pyridin-2-yl)thio)-1-(4-(4-(5-(2,6-dichlorophenyl)-4,5-dihydroisoxazol-3-yl)thiazol-2-yl)piperidin-1-yl)ethan-1-one; 1-(4-(4-(5-(2,6-dichlorophenyl)-4,5-dihydroisoxazol-3-yl)thiazol-2-yl)piperidin-1-yl)-2-((5-(trifluoromethyl)pyridin-2-yl)amino)ethan-1-one; 1-(4-(4-(5-(2,6-dichlorophenyl)-4,5-dihydroisoxazol-3-yl)thiazol-2-yl)piperidin-1-yl)-2-((3-methylpyridin-2-yl)oxy)ethan-1-one; 1-(4-(4-(5-(2,6-difluorophenyl)-4,5-dihydroisoxazol-3-yl)thiazol-2-yl)piperidin-1-yl)-2-((3-methylpyridin-2-yl)oxy)ethan-1-one; 3-chloro-2-(3-(2-(1-(2-((3-methylpyridin-2-yl)oxy)acetyl)piperidin-4-yl)thiazol-4-yl)-4,5-dihydroisoxazol-5-yl)phenylmethanesulfonate; 2-((3-methylpyridin-2-yl)oxy)-1-(4-(4-(5-(2,4,6-trichlorophenyl)-4,5-dihydroisoxazol-3-yl)thiazol-2-yl)piperdin-1-yl)ethan-1-one; 1-(4-(2-(5-(2,6-dichlorophenyl)-4,5-dihydroisoxazol-3-yl)pyridin-4-yl)piperidin-1-yl)-2-((3-(trifluoromethyl)pyridin-2-yl)oxy)ethan-1-one; 3-chloro-2-(3-(2-(1-(2-((3-chloro-5-(trifluoromethyl)pyridin-2-yl)thio)acetyl)piperidin-4-yl)thiazol-4-yl)-4,5-dihydroisoxazol-5-yl)phenylmethanesulfonate; 1-(4-(4-(5-(2,6-dichlorophenyl)-4,5-dihydroisoxazol-3-yl)thiazol-2-yl)piperidin-1-yl)-2-((3-(trifluoromethyl)pyridin-2-yl)amino)ethan-1-one; 2-((5-methyl-3-(trifluoromethyl)pyridin-2-yl)oxy)-1-(4-(4-(5-(2,4,6-trichlorophenyl)-4,5-dihydroisoxazol-3-yl)thiazol-2-yl)piperidin-1-yl)ethan-1-one; 1-(4-(4-(5-(2,6-dichlorophenyl)-4,5-dihydroisoxazol-3-yl)thiazol-2-yl)piperidin-1-yl)-2-((5-methyl-3-(trifluoromethyl)pyridin-2-yl)oxy)ethan-1-one; 3-chloro-2-(3-(2-(1-(2-((5-methyl-3-(trifluoromethyl)pyridin-2-yl)oxy)acetyl)piperidin-4-yl)thiazol-4-yl)-4,5-dihydroisoxazol-5-yl)phenylmethanesulfonate; 2-((3-(difluoromethyl)pyridin-2-yl)oxy)-1-(4-(4-(5-(2,6-difluorophenyl)-4,5-dihydroisoxazol-3-yl)thiazol-2-yl)piperidin-1-yl)ethan-1-one; 2-((3-(difluoromethyl)pyridin-2-yl)oxy)-1-(4-(4-(5-(2,4,6-trichlorophenyl)-4,5-dihydroisoxazol-3-yl)thiazol-2-yl)piperidin-1-yl)ethan-1-one; 1-(4-(4-(5-(2,6-difluorophenyl)-4,5-dihydroisoxazol-3-yl)thiazol-2-yl)piperidin-1-yl)-2-((5-methyl-3-(trifluoromethyl)pyridin-2-yl)oxy)ethan-1-one; 3-chloro-2-(3-(2-(1-(2-((3-(difluoromethyl)pyridin-2-yl)oxy)acetyl)piperidin-4-yl)thiazol-4-yl)-4,5-dihydroisoxazol-5-yl)phenylmethanesulfonate; 1-(4-(4-(5-(2,6-dichlorophenyl)-4,5-dihydroisoxazol-3-yl)thiazol-2-yl)piperidin-1 yl)-2-((3-(difluoromethyl)pyridin-2-yl)oxy)ethan-1-one; 3-(2-(1-(2-((3-(trifluoromethyl)pyridin-2-yl)oxy)acetyl)piperidin-4-yl)thiazol-4-yl)-1,5-dihydrobenzo[e][1,3]dioxepin-6-ylmethanesulfonate; and 3-(2-(1-(2-((6-methyl-3-(trifluoromethyl)pyridin-2-yl)oxy)acetyl)piperidin-4-yl)thiazol-4-yl)-1,5-dihydrobenzo[e][1,3]dioxepin-6-ylmethanesulfonate.

6. A composition for controlling or preventing phytopathogenic micro-organisms comprising;
a. the compound of Formula I as claimed in claim 1, and
b. one or more inert carriers.

7. The composition as claimed in claim 6, further comprises one or more active compatible compounds selected from fungicides, insecticides, nematicides, acaricides, biopesticides, herbicides, plant growth regulators, antibiotics, fertilizers and nutrients.

8. The composition as claimed in claim 6, wherein the concentration of the compound of Formula I ranges from 1 to 90% by weight with respect to the total weight of the composition, preferably from 5 to 50% by weight with respect to the total weight of the composition.

9. A combination comprising;
a. a compound of Formula I; and
b. one or more active compatible compounds selected from fungicides, insecticides, nematicides, acaricides, biopesticides, herbicides, plant growth regulators, antibiotics, fertilizers and nutrients.

10. Use of compound of Formula I as claimed claim 1, for controlling or preventing phytopathogenic fungi, stramenopiles, bacteria, insects, nematodes, trematodes and mites in agricultural crops and or horticultural crops.

11. Use of composition as claimed in claim 6, for controlling or preventing phytopathogenic fungi, stramenopiles, bacteria, insects, nematodes, tremadotes and mites in agricultural crops and or horticultural crops.

12. Use of combination as claimed in claim 9, for controlling or preventing phytopathogenic fungi, stramenopiles, bacteria, insects, nematodes, trematodes and mites in agricultural crops and or horticultural crops.

13. Use of compound as claimed in claim 10, for controlling or preventing phytopathogenic fungi and oomycetes in agricultural crops and or horticultural crops.

14. Use of the compound as claimed in claim 10, wherein the agricultural crops are selected from cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and other vegetables, and ornamentals.

15. A method of controlling or preventing infestation of useful plants by phytopathogenic micro-organisms in agricultural crops and or horticultural crops wherein the compound of Formula I claimed in claim 1, is applied to the plants, to parts thereof or the loci thereof.

16. A method of controlling or preventing infestation of useful plants by phytopathogenic micro-organisms in agricultural crops and or horticultural crops wherein the compound of Formula I claimed in claim 1, is applied to seeds.

17. A process for preparing compound of Formula I, wherein the process comprises step/s of:
a. reacting a compound of Formula 1 or 2 with a compound of Formula IN, 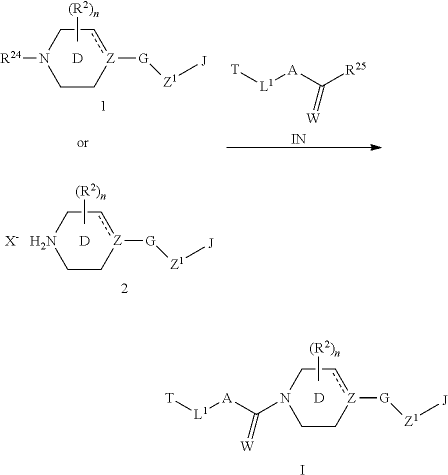
wherein, R²⁴ is hydrogen, or —OC(═O)C₁-C₆-alkyl; R²⁵ is hydroxy, chlorine, or —OC₁-C₆-alkyl; X⁻ is selected from HSO₄⁻, Cl⁻, Br⁻, I⁻, CH₃C(═O)O⁻, CF₃C(═O)O⁻; L¹ is O or S; and R², A, G, J, T, W, Z¹ and n are each as defined in claim 1; or
reacting the compound of Formula 2 or 3 with the compound of Formula IN′ to obtain a compound of Formula 4, 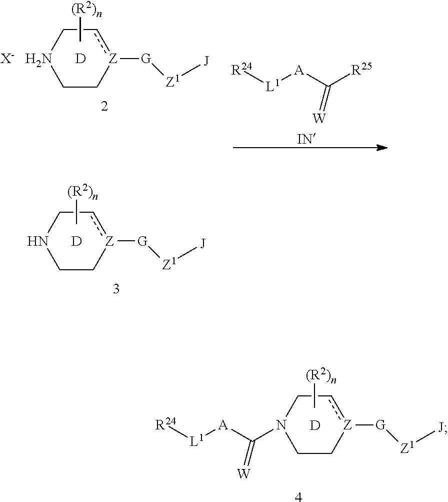
wherein, L¹ is N; R², R²⁴, R²⁵, A, G, J, W, X⁻, Z, Z¹ and n are each as defined herein above;
b. reacting the compound of Formula 4 with the compound of Formula IN″ to obtain the compound of Formula 1, 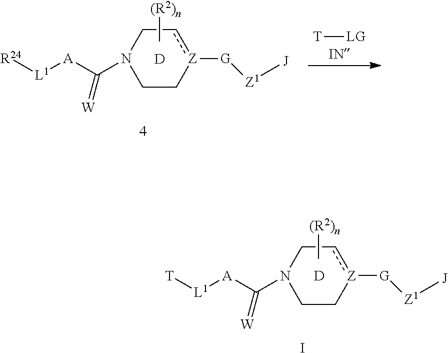
wherein, L¹ is N, LG is halogen; R², R²⁴, A, G, J, T, W, Z, Z¹ and n are each as defined herein above.

18. A compound of Formula 4: 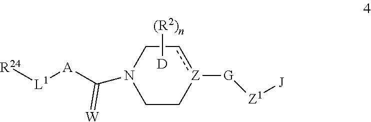
wherein, L¹ is N; R²⁴ is hydrogen, or —OC(═O)C₁-C₆-alkyl; R², A, G, J, W, Z, Z¹ and n are each as defined in claim 1.
